(19) United States
(12) Patent Application Publication
(10) Pub. No.: US 2018/0036315 A1
(43) Pub. Date: Feb. 8, 2018

(54) Oxazine Derivative for Use in the Prevention of Alzheimer's Disease in at Risk Patients

(71) Applicant: Novartis AG, Basel (CH)

(72) Inventors: Cristina LOPEZ-LOPEZ, Basel (CH); Ulf NEUMANN, Rheinfelden (CH)

(21) Appl. No.: 15/651,845

(22) Filed: Jul. 17, 2017

(30) Foreign Application Priority Data
Jul. 19, 2016 (EP) 16180233.5
Oct. 13, 2016 (EP) 16193779.2

(51) Int. Cl. A61K 31/5377

(52) U.S. Cl. CPC A61K 31/5377

(57) ABSTRACT

The present invention relates to an oxazine derivative BACE-1 inhibitor and pharmaceutical compositions comprising such oxazine derivative for use in the prevention of Alzheimer's disease in a patient at risk of developing clinical symptoms of Alzheimer's disease, and in particular, wherein the patient at risk of developing clinical symptoms of Alzheimer's disease carries one or two copies of the ApoE4 allele.

### FIELD OF THE INVENTION

The present invention relates to an oxazine derivative, and pharmaceutical compositions comprising such oxazine derivative, for use in the prevention of Alzheimer's disease in a patient at risk of developing clinical symptoms of Alzheimer's disease; and, in particular, where the patient at risk of developing clinical symptoms of Alzheimer's disease carries one or two copies of the ApoE4 allele.

### BACKGROUND TO THE INVENTION

Alzheimer's disease (AD) is one of the most prevalent neurological disorders worldwide and the most common and debilitating age-related condition, causing progressive amnesia, dementia, and ultimately global cognitive failure and death. Currently, the only pharmacological therapies available are symptomatic drugs such as cholinesterase inhibitors or other drugs used to control the secondary behavioral symptoms of AD. Investigational treatments targeting the AD pathogenic cascade include those intended to interfere with the production, accumulation, or toxic sequelae of amyloid-β (Aβ) species (Kramp V P, Herrling P, 2011). Strategies that target decreasing Aβ by: (1) enhancing the amyloid clearance with an active or passive immunotherapy against Aβ; (2) decreasing production through inhibition of Beta-site-APP cleaving enzyme-1 (BACE-1, an enzyme involved in the processing of the amyloid precursor protein [APP]), are of potential therapeutic value.

Based on animal data and limited benefits in recent clinical trials targeting dementia stages of the disease, there is a growing belief that the Aβ-lowering therapies might be most effective in preventing or slowing the progression of AD in the preclinical stages. This approach allows participants to be treated before, or in the very earliest stages of, symptoms and disease onset, prior to plateau of fibrillary Aβ, extensive appearance of tau (neurofibrillary) pathology and irreversible synaptic or neuronal loss.

The ε4 allele of the apolipoprotein E (ApoE4) gene is the main risk factor for Alzheimer's disease (AD). The APOE gene exists in three polymorphic alleles, ε2, ε3 and ε4, where ε3 is the most frequent. The APOE isoforms affect Aβ clearance, aggregation and deposition differently; ε2 seems to be protective whereas ε4 carriers have enhanced pathology and accelerated age-dependent cognitive decline (for review see Liu C C et al., 2013)).

Human ApoE is located on chromosome 19 (gene APOE, Uniprot P02649, gene codes for 317 amino acids, including a pro-peptide of 18 amino acids), the mature form is composed of 299 amino acids, and has 2 separate N-terminal and C-terminal domains joined by a flexible linker. While the N-terminal domain contains the binding domain for receptor binding (aa 136-150), the lipid binding domain (aa 240-260) is located in the C-terminal part. Three major isoforms (apoE2, -3 and -4) are known in humans, the allele frequency of ApoE3 (having Cys at position 112 and Arg at position at position 158) is approximately 50-90% in humans. ApoE2 (with Cys at positions 112 and 158) has an allele frequency of 1-5%, and ApoE4 (with Arg at positions 112 and 158) has an allele frequency of 5-35% in humans. ApoE3 and 4 bind to the LDL receptor with high affinity, while ApoE2 (due to the Cys-158) has only low affinity.

ApoE4 homozygotes are estimated to represent about 2 to 3% of the general population and are at much higher risk of developing symptoms of AD, with a mean age of 68 years at onset, than people with other APOE genotypes (Corder E H et al., 1993). By age 85, the lifetime risk of symptomatic AD may be as high as 51% for male homozygotes and 60-68% for female homozygotes. The corresponding percentage risks for 85 year old ApoE4 heterozygotes are 23% and 30% for males and females respectively carrying an ApoE3/4 genotypes and 20% and 27% for males and females respectively carrying an ApoE2/4 genotype (Genin E et al., 2011). It is proposed that the presence of the ApoE4 gene enhances the risk for AD by affecting Aβ clearance, aggregation, and deposition (Liu C C et al., 2013). It is expected that the presence of brain amyloid pathology in ApoE4 heterozygotes significantly increases the risk of developing clinical symptoms of AD comparable to homozygotes.

### SUMMARY OF THE INVENTION

The compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, referred to herein as “Compound 1”, is an orally active BACE inhibitor, previously described in WO 2012/095469 A1, with an approximately 3-fold selectivity for BACE-1 over BACE-2 and no relevant off-target binding or activity.

Given the high rate of setback and disappointment in the field to date (Cummings J L et al., 2014), there is a high degree of uncertainty as to whether any experimental disease-modifying AD therapy will prove effective in at-risk patients. However, the high degree of effectiveness demonstrated herein by Compound 1 in: (i) lowering Aβ levels in ApoE4 transgenic mice and human ApoE4 carriers, in the absence of undesirable side effects, for example hair discolouration; (ii) reducing amyloid-β deposition in the APP23 mouse model; and, especially, (iii) in raising the ratio of Aβ42/Aβ40 in cerebrospinal fluid, indicative of an effect on the underlying AD pathology; strongly suggests that Compound 1 will be effective in the prevention of AD in a patient at risk of developing clinical symptoms of AD, and in particular, those patients carrying one or two copies of the ApoE4 allele.

A Phase II/III clinical trial is described herein which has been designed to demonstrate the effectiveness of Compound 1 in the prevention of AD in cognitively unimpaired ApoE4 homozygote patients or cognitively unimpaired, amyloid positive, ApoE4 heterozygote patients. Based on current knowledge, the findings from this proposed clinical trial and the results described herein may be generalised and applicable to AD in at-risk patients beyond ApoE4 homozygotes and heterozygotes (for example in patients carrying mutations in the genes for amyloid precursor protein (APP), presenilin-1 and -2 (O'Brien R J, Wong P C, 2011) or in Down Syndrome patients (Head E et al., 2012)) since a BACE inhibitor therapy would be expected to reduce and/or prevent amyloid plaque accumulation independent of the multiple potential causes of amyloid deposition.

In a first aspect of the invention, there is therefore provided the compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof, for use in the prevention of Alzheimer's disease in a patient at risk of developing clinical symptoms of Alzheimer's disease.

In a second aspect of the invention, there is provided a pharmaceutical composition comprising N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof, for use in the prevention of Alzheimer's disease in a patient at risk of developing clinical symptoms of Alzheimer's disease.

In a third aspect of the invention, there is provided a method for the prevention of Alzheimer's disease in a patient at risk of developing clinical symptoms of Alzheimer's disease which method comprises administering to such patient a therapeutically effective amount of the compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof.

In a fourth aspect of the invention, there is provided a method for the prevention of Alzheimer's disease in a patient at risk of developing clinical symptoms of Alzheimer's disease which method comprises administering to such patient a pharmaceutical composition comprising a therapeutically effective amount of the compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof.

In a fifth aspect of the invention, there is provided the use of the compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof, for the prevention of Alzheimer's disease in a patient at risk of developing clinical symptoms of Alzheimer's disease.

In a sixth aspect of the invention, there is provided the use of a pharmaceutical composition comprising the compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof, for the prevention of Alzheimer's disease in a patient at risk of developing clinical symptoms of Alzheimer's disease.

In a seventh aspect of the invention, there is provided the use of the compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof, for the manufacture of a medicament for the prevention of Alzheimer's disease in a patient at risk of developing clinical symptoms of Alzheimer's disease.

### DESCRIPTION OF THE INVENTION

### List of Figures

FIG. 1: Fur colour scores of C57BL/6 mice chronically treated with Compound 1 or NB-360 for 8 weeks (mean±SEM)

FIG. 2: Reduction of brain Aβ40 in C57BL/6 mice upon treatment with Compound 1 at 8 and 50 μmol/kg after last dose (mean±SD, n=4 per group)

FIG. 3: Effect of acute administration of Compound 1 on Forebrain Aβ40 levels in APOE4-TR male and female mice (3-5 month-old, Mean±SEM)

FIG. 4: Effect of acute administration of Compound 1 on CSF Aβ40 levels in APOE4-TR male and female mice (3-5 month-old) (Mean±SEM)

FIG. 5: Effect of acute administration of Compound 1 on CSF Aβ42 levels in APOE4-TR male and female mice (3-5 month-old) (Mean±SEM)

FIG. 6: Compound 1 acute exposure in APOE4-TR male and female mice (3-5 month-old, Mean±SD)

FIG. 7: Brain PK/PD relationship (individual data)

FIG. 8: Brain PK/PD relationship (Mean±SD)

FIG. 9: Effect of Compound 1 on CSF Aβ40 levels after two-week exposure in multiple ascending oral dose study in human subjects

FIG. 10: Effect of Compound 1 on CSF Aβ40 levels in human subjects—% change from baseline at 3 months (24 hours post last dose)

FIG. 11: Effect of Compound 1 on Aβ40 in Triton TX-100 extracted APP23 brains

FIG. 12: Effect of Compound 1 on Aβ42 in Triton TX-100 extracted APP23 brains

FIG. 13: Effect of Compound 1 on sAPPα in Triton TX-100 extracted APP23 brains

FIG. 14: Effect of Compound 1 on sAPPβ (Swe) in Triton TX-100 extracted APP23 brains

FIG. 15: Effects of Compound 1 treatment on Aβ40 in the cerebrospinal fluid of APP23 mice

FIG. 16: Effect of Compound 1 on formic acid soluble Aβ40 in mouse (values are mean±SEM)

FIG. 17: Effect of Compound 1 on formic acid soluble Aβ42 in mouse (values are mean±SEM)

FIG. 18: Effect of Compound 1 on formic acid soluble total Aβ (40+42) in mouse (values are mean±SEM)

FIG. 19: Effect of Compound 1 on formic acid soluble Aβ42/40 ratio in mouse (values are mean±SEM)

FIG. 20: Effect of Compound 1 on plaque histology—number of small plaques (data normalized to total area)

FIG. 21: Effect of Compound 1 on plaque histology—number of medium plaques (data normalized to total area)

FIG. 22: Effect of Compound 1 on plaque histology—number of large plaques (data normalized to total area)

FIG. 23: Effect of Compound 1 on plaque histology—total plaque area (data normalized to total area)

FIG. 24: Total GFAP positive area, normalized for total area. Shown are means±SEM. Comparison was performed with Dunnett's multiple comparison test.

FIG. 25: Plaque-associated GFAP positive area, normalized for total area. Shown are means±SEM. Comparison was performed with Dunnett's multiple comparison test.

FIG. 26: Non-plaque-associated GFAP positive area, normalized for total area. Shown are means±SEM. Comparison was performed with Dunnett's multiple comparison test.

FIG. 27: Proximal GFAP positive area, normalized for total area. Shown are means±SEM. Comparison was performed with Dunnett's multiple comparison test.

FIG. 28: Distal GFAP positive area, normalized for total area. Shown are means±SEM. Comparison was performed with Dunnett's multiple comparison test.

FIG. 29: Effect of Compound 1 treatment on total IBA1 positive area. Shown are distinct microglia populations, normalized by sample area. Shown are means±SEM. Comparison was performed with Dunnett's multiple comparison test.

FIG. 30: Effect of Compound 1 treatment on plaque-associated IBA1 positive area. Shown are distinct microglia populations, normalized by sample area. Shown are means±SEM. Comparison was performed with Dunnett's multiple comparison test.

FIG. 31: Effect of Compound 1 treatment on non-plaque-associated IBA1+ area. Shown are distinct microglia populations, normalized by sample area. Shown are means±SEM. Comparison was performed with Dunnett's multiple comparison test.

FIG. 32: Effect of Compound 1 treatment on proximal IBA1+ area. Shown are distinct microglia populations, normalized by sample area. Shown are means±SEM. Comparison was performed with Dunnett's multiple comparison test.

FIG. 33: Effect of Compound 1 treatment on distal IBA1+ area. Shown are distinct microglia populations, normalized by sample area. Shown are means±SEM. Comparison was performed with Dunnett's multiple comparison test.

FIG. 34: Design of a two part, open-label, two-period, fixed-sequence study in healthy subjects to evaluate the PK of Compound 1 when given alone and in combination with the strong CYP3A4 inhibitor itraconazole or the strong CYP3A4 inducer rifampicin.

FIG. 35: Fold change from baseline of CSF Aβ42/Aβ40 ratio in response to treatment with Compound 1 in non-ApoE4 carrier and ApoE4 carrier healthy elderly subjects having an CSF Aβ42/Aβ40 ratio<0.09 at baseline. Comparison was performed with Dunnett's multiple comparison test.

Various Embodiments of the present invention are herein described.

### SERIES A EMBODIMENTS OF THE FIRST ASPECT OF THE INVENTION

### Embodiment A1

The compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof, for use in the prevention of Alzheimer's disease in a patient at risk of developing clinical symptoms of Alzheimer's disease.

### Embodiment A2

The compound, or a pharmaceutically acceptable salt thereof, for the use according to Embodiment A1, wherein the patient at risk of developing clinical symptoms of Alzheimer's disease carries a genetic predisposition for the development of the clinical symptoms of Alzheimer's disease or has Down syndrome.

### Embodiment A3

The compound, or a pharmaceutically acceptable salt thereof, for the use according to Embodiment A2, wherein the patient carries a genetic predisposition for the development of the clinical symptoms of Alzheimer's disease and the genetic predisposition is:
(i) a mutation in the gene for amyloid precursor protein, presenilin-1 or presenilin-2; or
(ii) the presence of one or two copies of the ApoE4 allele.

### Embodiment A4

The compound, or a pharmaceutically acceptable salt thereof, for the use according to Embodiment A3, wherein the patient at risk of developing clinical symptoms of Alzheimer's disease carries one or two copies of the ApoE4 allele.

### Embodiment A5

The compound, or a pharmaceutically acceptable salt thereof, for the use according to Embodiment A4, wherein the patient carries one copy of the ApoE4 allele.

### Embodiment A6

The compound, or a pharmaceutically acceptable salt thereof, for the use according to Embodiment A4, wherein the patient carries two copies of the ApoE4 allele.

### Embodiment A7

The compound, or a pharmaceutically acceptable salt thereof, for the use according to any one of Embodiments A1 to A6, wherein the patient is amyloid-positive.

### Embodiment A8

The compound, or a pharmaceutically acceptable salt thereof, for the use according to Embodiment A7, wherein the amyloid-positivity is determined by PET or CSF measurement.

### Embodiment A9

The compound, or a pharmaceutically acceptable salt thereof, for the use according to any one of Embodiments A3 to A8, wherein the patient is between 60 and 75 years of age.

### Embodiment A10

The compound, or a pharmaceutically acceptable salt thereof, for the use according to any one of Embodiments A1 to A9, wherein the compound is used at a daily dose which results in at least a 70% lowering of Aβ1-40 in CSF following two weeks of compound exposure.

Embodiment A11: The compound, or a pharmaceutically acceptable salt thereof, for the use according to any one of Embodiments A1 to A9, wherein the compound is used at a daily dose which results in at least a 50% lowering of Aβ1-40 in CSF following two weeks of compound exposure.

Embodiment A12: The compound, or a pharmaceutically acceptable salt thereof, for the use according to any one of Embodiments A1 to A9, wherein the compound is used at a dose of between 10 and 30 mg per day.

### Embodiment A13

The compound, or a pharmaceutically acceptable salt thereof, for the use according to any one of Embodiments A1 to A9, wherein the compound is used at a dose of between 30 and 50 mg per day.

### Embodiment A14

The compound, or a pharmaceutically acceptable salt thereof, for the use according to any one of Embodiments A1 to A9, wherein the compound is used at a dose of 15 mg per day.

### Embodiment A15

The compound, or a pharmaceutically acceptable salt thereof, for the use according to any one of Embodiments A1 to A9, wherein the compound is used at a dose of 50 mg per day.

### Embodiment A16

The compound, or a pharmaceutically acceptable salt thereof, for the use according to any one of Embodiments A1 to A9, wherein the compound is used at a daily dose which results in a plasma steady state Cmax value of between 70 and 170 ng/ml.

### Embodiment A17

The compound, or a pharmaceutically acceptable salt thereof, for the use according to any one of Embodiments A1 to A9, wherein the compound is used at a daily dose which results in a plasma steady state Cmax value of between 200 and 500 ng/ml.

### Embodiment A18

The compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof, for use in the prevention of Alzheimer's disease in a patient at risk of developing clinical symptoms of Alzheimer's disease, wherein the patient at risk of developing clinical symptoms of Alzheimer's disease carries one or two copies of the ApoE4 allele.

### Embodiment A19

The compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof, for use in the prevention of Alzheimer's disease in a patient at risk of developing clinical symptoms of Alzheimer's disease, wherein the patient at risk of developing clinical symptoms of Alzheimer's disease carries one or two copies of the ApoE4 allele, and wherein the compound is used at a dose of 15 or 50 mg per day.

### Embodiment A20

The compound for the use according to any one of Embodiments A1 to A19, wherein the compound is in free form.

### Embodiment A21

The compound, or a pharmaceutically acceptable salt thereof, for the use according to any one of Embodiments A1 to A20, wherein the patient is not simultaneously treated with an inhibitor or inducer of CYP3A4.

### Embodiment A22

The compound, or a pharmaceutically acceptable salt thereof, for the use according to any one of Embodiments A1 to A20, wherein the patient is not simultaneously treated with a CYP3A4 inhibitor or inducer for a period longer than three months.

### Embodiment A23

The compound, or a pharmaceutically acceptable salt thereof, for the use according to Embodiment A21 or A22, wherein the CYP3A4 inhibitor is a strong, moderate, or weak inhibitor of CYP3A4; and the CYP3A4 inducer is a strong, moderate, or weak inducer of CYP3A4.

### Embodiment A24

The compound, or a pharmaceutically acceptable salt thereof, for the use according to Embodiment A23, wherein the CYP3A4 inhibitor is a strong inhibitor of CYP3A4; and the CYP3A4 inducer is a strong inducer of CYP3A4.

### SERIES B EMBODIMENTS OF THE SECOND ASPECT OF THE INVENTION

### Embodiment B1

A pharmaceutical composition comprising the compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof, for use in the prevention of Alzheimer's disease in a patient at risk of developing clinical symptoms of Alzheimer's disease.

### Embodiment B2

The pharmaceutical composition for the use according to Embodiment B1, wherein the patient at risk of developing clinical symptoms of Alzheimer's disease carries a genetic predisposition for the development of the clinical symptoms of Alzheimer's disease or has Down syndrome.

### Embodiment B3

The pharmaceutical composition for the use according to Embodiment B2, wherein the patient carries a genetic predisposition for the development of the clinical symptoms of Alzheimer's disease and the genetic predisposition is:
(i) a mutation in the gene for amyloid precursor protein, presenilin-1 or presenilin-2; or
(ii) the presence of one or two copies of the ApoE4 allele.

### Embodiment B4

The pharmaceutical composition for the use according to Embodiment B3, wherein the patient at risk of developing clinical symptoms of Alzheimer's disease carries one or two copies of the ApoE4 allele.

### Embodiment B5

The pharmaceutical composition for the use according to Embodiment B4, wherein the patient carries one copy of the ApoE4 allele.

### Embodiment B6

The pharmaceutical composition for the use according to Embodiment B4, wherein the patient carries two copies of the ApoE4 allele.

### Embodiment B7

The pharmaceutical composition for the use according to any one of Embodiments B1 to B6, wherein the patient is amyloid-positive.

### Embodiment B8

The pharmaceutical composition for the use according to Embodiment B7, wherein the amyloid-positivity is determined by PET or CSF measurement.

### Embodiment B9

The pharmaceutical composition for the use according to any one of Embodiments B3 to B8, wherein the patient is between 60 and 75 years of age.

### Embodiment B10

The pharmaceutical composition for the use according to any one of Embodiments B1 to B9, wherein the compound is used at a daily dose which results in at least a 70% lowering of Aβ1-40 in CSF following two weeks of compound exposure.

### Embodiment B11

The pharmaceutical composition for the use according to any one of Embodiments B1 to B9, wherein the compound is used at a daily dose which results in at least a 50% lowering of Aβ1-40 in CSF following two weeks of compound exposure.

### Embodiment B12

The pharmaceutical composition for the use according to any one of Embodiments B1 to B9, wherein the compound is used at a dose of between 10 and 30 mg per day.

### Embodiment B13

The pharmaceutical composition for the use according to any one of Embodiments B1 to B9, wherein the compound is used at a dose of between 30 and 50 mg per day.

### Embodiment B14

The pharmaceutical composition for the use according to any one of Embodiments B1 to B9, wherein the compound is used at a dose of 15 mg per day.

### Embodiment B15

The pharmaceutical composition for the use according to any one of Embodiments B1 to B9, wherein the compound is used at a dose of 50 mg per day.

### Embodiment B16

The pharmaceutical composition for the use according to any one of Embodiments B1 to B9, wherein the compound is used at a daily dose which results in a plasma steady state Cmax value of between 70 and 170 ng/ml.

### Embodiment B17

The pharmaceutical composition for the use according to any one of Embodiments B1 to B9, wherein the compound is used at a daily dose which results in a plasma steady state Cmax value of between 200 and 500 ng/ml.

### Embodiment B18

A pharmaceutical composition comprising the compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof, for use in the prevention of Alzheimer's disease in a patient at risk of developing clinical symptoms of Alzheimer's disease, wherein the patient at risk of developing clinical symptoms of Alzheimer's disease carries one or two copies of the ApoE4 allele.

### Embodiment B19

A pharmaceutical composition comprising the compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof, for use in the prevention of Alzheimer's disease in a patient at risk of developing clinical symptoms of Alzheimer's disease, wherein the patient at risk of developing clinical symptoms of Alzheimer's disease carries one or two copies of the ApoE4 allele, and wherein the compound is used at a dose of 15 or 50 mg per day.

### Embodiment B20

The pharmaceutical composition for the use according to any one of Embodiments B1 to B19, wherein the compound is in free form.

### Embodiment B21

The pharmaceutical composition for the use according to any one of Embodiments B1 to B20, wherein the patient is not simultaneously treated with an inhibitor or inducer of CYP3A4.

### Embodiment B22

The pharmaceutical composition for the use according to any one of Embodiments B1 to B20, wherein the patient is not simultaneously treated with a CYP3A4 inhibitor or inducer for a period longer than three months.

### Embodiment B23

The pharmaceutical composition for the use according to Embodiment B21 or B22, wherein the CYP3A4 inhibitor is a strong, moderate, or weak inhibitor of CYP3A4; and the CYP3A4 inducer is a strong, moderate, or weak inducer of CYP3A4.

### Embodiment B24

The pharmaceutical composition for the use according to Embodiment B23, wherein the CYP3A4 inhibitor is a strong inhibitor of CYP3A4; and the CYP3A4 inducer is a strong inducer of CYP3A4.

### SERIES C EMBODIMENTS OF THE THIRD ASPECT OF THE INVENTION

### Embodiment C1

A method for the prevention of Alzheimer's disease in a patient at risk of developing clinical symptoms of Alzheimer's disease which method comprises administering to such patient a therapeutically effective amount of the compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof.

### Embodiment C2

The method according to Embodiment C1, wherein the patient at risk of developing clinical symptoms of Alzheimer's disease carries a genetic predisposition for the development of the clinical symptoms of Alzheimer's disease or has Down syndrome.

### Embodiment C3

The method according to Embodiment C2, wherein the patient carries a genetic predisposition for the development of the clinical symptoms of Alzheimer's disease and the genetic predisposition is:
(i) a mutation in the gene for amyloid precursor protein, presenilin-1 or presenilin-2; or
(ii) the presence of one or two copies of the ApoE4 allele.

### Embodiment C4

The method according to Embodiment C3, wherein the patient at risk of developing clinical symptoms of Alzheimer's disease carries one or two copies of the ApoE4 allele.

### Embodiment C5

The method according to Embodiment C4, wherein the patient carries one copy of the ApoE4 allele.

### Embodiment C6

The method according to Embodiment C4, wherein the patient carries two copies of the ApoE4 allele.

### Embodiment C7

The method according to any one of Embodiments C1 to C6, wherein the patient is amyloid-positive.

### Embodiment C8

The method according to Embodiment C7, wherein the amyloid-positivity is determined by PET or CSF measurement.

### Embodiment C9

The method according to any one of Embodiments C3 to C8, wherein the patient is over 60, 61, 62, 63, 64, 65, 66, 67, 68, 69, 70, 71, 72, 73, 74 or 75 years of age.

### Embodiment C10

The method according to any one of Embodiments C3 to C8, wherein the patient is between 60 and 75 years of age.

### Embodiment C11

The method according to any one of Embodiments C1 to C10, wherein the compound is used at a daily dose which results in at least 10, 20, 30, 40, 50, 60, 70 or 80% lowering of Aβ 1-40 in CSF, blood, or plasma, following 2, 13, 26, 52, 78, 104, 130, 156, 182, 208, 234, 260, 286, 312, 338, 332, 390, or 416 weeks of compound exposure.

### Embodiment C12

The method according to any one of Embodiments C1 to C10, wherein the compound is used at a daily dose which results in at least a 70% lowering of Aβ1-40 in CSF, blood, or plasma, following 2, 13, 26, 52, 78, 104, 130, 156, 182, 208, 234, 260, 286, 312, 338, 332, 390, or 416 weeks of compound exposure.

### Embodiment C13

The method according to any one of Embodiments C1 to C10, wherein the compound is used at a daily dose which results in at least a 50% lowering of Aβ1-40 in CSF, blood, or plasma, following 2, 13, 26, 52, 78, 104, 130, 156, 182, 208, 234, 260, 286, 312, 338, 332, 390, or 416 weeks of compound exposure.

### Embodiment C14

The method according to any one of Embodiments C1 to C10, wherein the compound is used at a daily dose which results in a lowering of Aβ1-40 in CSF, blood or plasma, in the range of 10, 20, 30, 40, 50, 60, 70 or 80% to 99, 97, 95, 93, 90, 87, 85, 80, 75, 70, 65, 60, 55, or 50%, following 2, 13, 26, 52, 78, 104, 130, 156, 182, 208, 234, 260, 286, 312, 338, 332, 390, or 416 weeks of compound exposure.

### Embodiment C15

The method according to any one of Embodiments C1 to C10, wherein the compound is used at a daily dose which results in a lowering of Aβ1-40 in CSF, blood or plasma, in the range of 40 to 70%, 45 to 65%, or 50 to 60%, or of at least 50% in at least 80, 85, 90, 93, 95, 97, or 99% of the patients or in 80, 85, or 90 to 99, 97, 95, or 93% of the patients.

### Embodiment C16

The method according to any one of Embodiments C1 to C10, wherein the compound is used at a daily dose which results in a lowering of Aβ1-40 in CSF, blood or plasma, in the range of 65 to 95%, 75 to 90%, or 80 to 90%, or of at least 80% in at least 80, 85, 90, 93, 95, 97, or 99% of the patients or in 80, 85, or 90 to 99, 97, 95, or 93% of the patients.

### Embodiment C17

The method according to any one of Embodiments C1 to C10, wherein the compound is used at a dose of between 5 and 10; 10 and 15; 15 and 20; 20 and 25; 25 and 30; 30 and 35; 35 and 40; 45 and 50; 50 and 55 mg; 55 and 60 mg; 60 and 100 mg; 100 and 200; 200 and 300 mg; 15 and 85 mg; 50 and 85 mg; 15 and 300 mg; or 50 and 300 mg per day.

### Embodiment C18

The method according to any one of Embodiments C1 to C10, wherein the compound is used at a dose of between 10 and 30 mg per day.

### Embodiment C19

The method according to any one of Embodiments C1 to C10, wherein the compound is used at a dose of between 30 and 50 mg per day.

### Embodiment C20

The method according to any one of Embodiments C1 to C10, wherein the compound is used at a dose of 15 mg per day.

### Embodiment C21

The method according to any one of Embodiments C1 to C10, wherein the compound is used at a dose of 50 mg per day.

### Embodiment C22

The method according to any one of Embodiments C1 to C10, wherein the compound is used at a daily dose which results in a plasma steady state Cmax value of between 0 and 50; 50 and 100; 100 and 150; 150 and 200; 200 and 250; 250 and 300; 300 and 350; 350 and 400; 400 and 450; 450 and 500; 500 and 550; 550 and 600; 600 and 650; or 650 and 700 ng/ml.

### Embodiment C23

The method according to any one of Embodiments C1 to C10, wherein the compound is used at a daily dose which results in a plasma steady state Cmax value of between 70 and 170 ng/ml.

### Embodiment C24

The method according to any one of Embodiments C1 to C10, wherein the compound is used at a daily dose which results in a plasma steady state Cmax value of between 200 and 500 ng/ml.

### Embodiment C25

A method for the prevention of Alzheimer's disease in a patient at risk of developing clinical symptoms of Alzheimer's disease which method comprises administering to such patient a therapeutically effective amount of the compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof, wherein the patient at risk of developing clinical symptoms of Alzheimer's disease carries one or two copies of the ApoE4 allele.

### Embodiment C26

A method for the prevention of Alzheimer's disease in a patient at risk of developing clinical symptoms of Alzheimer's disease which method comprises administering to such patient a therapeutically effective amount of the compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof, wherein the patient at risk of developing clinical symptoms of Alzheimer's disease carries one or two copies of the ApoE4 allele, and wherein the compound is used at a dose of 15 or 50 mg per day.

### Embodiment C27

The method according to any one of Embodiments C1 to C26, wherein the compound is in free form.

### Embodiment C28

The method according to any one of Embodiments C1 to C27 wherein Compound 1 is comprised within a pharmaceutical composition.

### Embodiment C29

The method according to any one of Embodiments C1 to C28, wherein the patient is not simultaneously treated with an inhibitor or inducer of CYP3A4.

### Embodiment C30

The method according to any one of Embodiments C1 to C28, wherein the patient is not simultaneously treated with a CYP3A4 inhibitor or inducer for a period longer than three months.

### Embodiment C31

The method according to Embodiment C29 or C30, wherein the CYP3A4 inhibitor is a strong, moderate, or weak inhibitor of CYP3A4; and the CYP3A4 inducer is a strong, moderate, or weak inducer of CYP3A4.

### Embodiment C32

The method according to Embodiment C31, wherein the CYP3A4 inhibitor is a strong inhibitor of CYP3A4; and the CYP3A4 inducer is a strong inducer of CYP3A4.

### SERIES D EMBODIMENTS OF THE FIFTH ASPECT OF THE INVENTION

### Embodiment D1

Use of the compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof, for the prevention of Alzheimer's disease in a patient at risk of developing clinical symptoms of Alzheimer's disease.

### Embodiment D2

The use according to Embodiment D1, wherein the patient at risk of developing clinical symptoms of Alzheimer's disease carries a genetic predisposition for the development of the clinical symptoms of Alzheimer's disease or has Down syndrome.

### Embodiment D3

The use according to Embodiment D2, wherein the patient carries a genetic predisposition for the development of the clinical symptoms of Alzheimer's disease and the genetic predisposition is:
(i) a mutation in the gene for amyloid precursor protein, presenilin-1 or presenilin-2; or
(ii) the presence of one or two copies of the ApoE4 allele.

### Embodiment D4

The use according to Embodiment D3, wherein the patient at risk of developing clinical symptoms of Alzheimer's disease carries one or two copies of the ApoE4 allele.

### Embodiment D5

The use according to Embodiment D4, wherein the patient carries one copy of the ApoE4 allele.

### Embodiment D6

The use according to Embodiment D4, wherein the patient carries two copies of the ApoE4 allele.

### Embodiment D7

The use according to any one of Embodiments D1 to D6, wherein the patient is amyloid-positive.

### Embodiment D8

The use according to Embodiment D7, wherein the amyloid-positivity is determined by PET or CSF measurement.

### Embodiment D9

The use according to any one of Embodiments D3 to D8, wherein the patient is between 60 and 75 years of age.

### Embodiment D10

The use according to any one of Embodiments D1 to D9, wherein the compound is used at a daily dose which results in at least a 70% lowering of Aβ1-40 in CSF following two weeks of compound exposure.

### Embodiment D11

The use according to any one of Embodiments D1 to D9, wherein the compound is used at a daily dose which results in at least a 50% lowering of Aβ1-40 in CSF following two weeks of compound exposure.

### Embodiment D12

The use according to any one of Embodiments D1 to D9, wherein the compound is used at a dose of between 10 and 30 mg per day.

### Embodiment D13

The use according to any one of Embodiments D1 to D9, wherein the compound is used at a dose of between 30 and 50 mg per day.

### Embodiment D14

The use according to any one of Embodiments D1 to D9, wherein the compound is used at a dose of 15 mg per day.

### Embodiment D15

The use according to any one of Embodiments D1 to D9, wherein the compound is used at a dose of 50 mg per day.

### Embodiment D16

The use according to any one of Embodiments D1 to D9, wherein the compound is used at a daily dose which results in a plasma steady state Cmax value of between 70 and 170 ng/ml.

### Embodiment D17

The use according to any one of Embodiments D1 to D9, wherein the compound is used at a daily dose which results in a plasma steady state Cmax value of between 200 and 500 ng/ml.

### Embodiment D18

Use of the compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof, for the prevention of Alzheimer's disease in a patient at risk of developing clinical symptoms of Alzheimer's disease, wherein the patient at risk of developing clinical symptoms of Alzheimer's disease carries one or two copies of the ApoE4 allele.

### Embodiment D19

Use of the compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof, for the prevention of Alzheimer's disease in a patient at risk of developing clinical symptoms of Alzheimer's disease, wherein the patient at risk of developing clinical symptoms of Alzheimer's disease carries one or two copies of the ApoE4 allele, and wherein the compound is used at a dose of 15 or 50 mg per day.

### Embodiment D20

The use according to any one of Embodiments D1 to D19, wherein the compound is in free form.

### Embodiment D21

The use according to any one of Embodiments D1 to D20, wherein the compound is comprised within a pharmaceutical composition.

### Embodiment D22

The use according to any one of Embodiments D1 to D21, wherein the patient is not simultaneously treated with an inhibitor or inducer of CYP3A4.

### Embodiment D23

The use according to any one of Embodiments D1 to D21, wherein the patient is not simultaneously treated with a CYP3A4 inhibitor or inducer for a period longer than three months.

### Embodiment D24

The use according to Embodiment D22 or D23, wherein the CYP3A4 inhibitor is a strong, moderate, or weak inhibitor of CYP3A4; and the CYP3A4 inducer is a strong, moderate, or weak inducer of CYP3A4.

### Embodiment D25

The use according to Embodiment D24, wherein the CYP3A4 inhibitor is a strong inhibitor of CYP3A4; and the CYP3A4 inducer is a strong inducer of CYP3A4.

### SERIES E EMBODIMENTS OF THE SEVENTH ASPECT OF THE INVENTION

### Embodiment E1

Use of the compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof, for the manufacture of a medicament for the prevention of Alzheimer's disease in a patient at risk of developing clinical symptoms of Alzheimer's disease.

### Embodiment E2

The use according to Embodiment E1, wherein the patient at risk of developing clinical symptoms of Alzheimer's disease carries a genetic predisposition for the development of the clinical symptoms of Alzheimer's disease or has Down syndrome.

### Embodiment E3

The use according to Embodiment E2, wherein the patient carries a genetic predisposition for the development of the clinical symptoms of Alzheimer's disease and the genetic predisposition is:
(i) a mutation in the gene for amyloid precursor protein, presenilin-1 or presenilin-2; or
(ii) the presence of one or two copies of the ApoE4 allele.

### Embodiment E4

The use according to Embodiment E3, wherein the patient at risk of developing clinical symptoms of Alzheimer's disease carries one or two copies of the ApoE4 allele.

### Embodiment E5

The use according to Embodiment E4, wherein the patient carries one copy of the ApoE4 allele.

### Embodiment E6

The use according to Embodiment E4, wherein the patient carries two copies of the ApoE4 allele.

### Embodiment E7

The use according to any one of Embodiments E1 to E6, wherein the patient is amyloid-positive.

### Embodiment E8

The use according to Embodiment E7, wherein the amyloid-positivity is determined by PET or CSF measurement.

### Embodiment E9

The use according to any one of Embodiments E3 to E8, wherein the patient is between 60 and 75 years of age.

### Embodiment E10

The use according to any one of Embodiments E1 to E9, wherein the compound is used at a daily dose which results in at least a 70% lowering of Aβ1-40 in CSF following two weeks of compound exposure.

Embodiment E11: The use according to any one of Embodiments E1 to E9, wherein the compound is used at a daily dose which results in at least a 50% lowering of Aβ1-40 in CSF following two weeks of compound exposure.

### Embodiment E12

The use according to any one of Embodiments E1 to E9, wherein the compound is used at a dose of between 10 and 30 mg per day.

### Embodiment E13

The use according to any one of Embodiments E1 to E9, wherein the compound is used at a dose of between 30 and 50 mg per day.

### Embodiment E14

The use according to any one of Embodiments E1 to E9, wherein the compound is used at a dose of 15 mg per day.

### Embodiment E15

The use according to any one of Embodiments E1 to E9, wherein the compound is used at a dose of 50 mg per day.

### Embodiment E16

The use according to any one of Embodiments E1 to E9, wherein the compound is used at a daily dose which results in a plasma steady state Cmax value of between 70 and 170 ng/ml.

### Embodiment E17

The use according to any one of Embodiments E1 to E9, wherein the compound is used at a daily dose which results in a plasma steady state Cmax value of between 200 and 500 ng/ml.

### Embodiment E18

Use of the compound N-(6-((3R,6R)-5-amino-3,6-dimethyl-6-(trifluoromethyl)-3,6-dihydro-2H-1,4-oxazin-3-yl)-5-fluoropyridin-2-yl)-3-ch loro-5-(trifluoromethyl)picolinamide, or a pharmaceutically acceptable salt thereof, for the manufacture of a medicament for the prevention of Alzheimer's disease in a patient at risk of developing clinical symptoms of Alzheimer's disease, wherein the patient at risk of developing clinical symptoms of Alzheimer's disease carries one or two copies of the ApoE4 allele.

### Embodiment E19

Use of the compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof, for the manufacture of a medicament for the prevention of Alzheimer's disease in a patient at risk of developing clinical symptoms of Alzheimer's disease, wherein the patient at risk of developing clinical symptoms of Alzheimer's disease carries one or two copies of the ApoE4 allele, and wherein the compound is used at a dose of 15 or 50 mg per day.

### Embodiment E20

The use according to any one of Embodiments E1 to E19, wherein the compound is in free form.

### Embodiment E21

The use according to any one of Embodiments E1 to E20, wherein the medicament is a pharmaceutical composition.

### Embodiment E22

The use according to any one of Embodiments E1 to E21, wherein the patient is not simultaneously treated with an inhibitor or inducer of CYP3A4.

### Embodiment E23

The use according to any one of Embodiments E1 to E21, wherein the patient is not simultaneously treated with a CYP3A4 inhibitor or inducer for a period longer than three months.

### Embodiment E24

The use according to Embodiment E22 or E23, wherein the CYP3A4 inhibitor is a strong, moderate, or weak inhibitor of CYP3A4; and the CYP3A4 inducer is a strong, moderate, or weak inducer of CYP3A4.

### Embodiment E25

The use according to Embodiment E24, wherein the CYP3A4 inhibitor is a strong inhibitor of CYP3A4; and the CYP3A4 inducer is a strong inducer of CYP3A4.

In a further invention, there is provided a method for the treatment or prevention of Alzheimer's disease which method comprises administering to a patient in need thereof a therapeutically effective amount of the compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof, wherein the patient is not simultaneously treated with an inhibitor or inducer of CYP3A4. In one embodiment, the patient is not simultaneously treated with an inhibitor or inducer of CYP3A4 for a period longer than three months. In one embodiment, the patient is simultaneously treated with a CYP3A4 inhibitor or inducer for a period no longer than three months. In one embodiment, the CYP3A4 inhibitor is a strong, moderate, or weak inhibitor of CYP3A4; and the CYP3A4 inducer is a strong, moderate, or weak inducer of CYP3A4. In one embodiment, the CYP3A4 inhibitor is a strong inhibitor of CYP3A4; and the CYP3A4 inducer is a strong inducer of CYP3A4. In one embodiment, the patient is over 60, 61, 62, 63, 64, 65, 66, 67, 68, 69, 70, 71, 72, 73, 74 or 75 years of age. In one embodiment, the patient is between 60 and 75 years of age. In one embodiment, the compound is used at a daily dose which results in at least 10, 20, 30, 40, 50, 60, 70 or 80% lowering of Aβ 1-40 in CSF, blood, or plasma, following 2, 13, 26, 52, 78, 104, 130, 156, 182, 208, 234, 260, 286, 312, 338, 332, 390, or 416 weeks of compound exposure. In one embodiment, the compound is used at a daily dose which results in at least a 70% lowering of Aβ1-40 in CSF, blood, or plasma, following 2, 13, 26, 52, 78, 104, 130, 156, 182, 208, 234, 260, 286, 312, 338, 332, 390, or 416 weeks of compound exposure. In one embodiment, the compound is used at a daily dose which results in at least a 50% lowering of Aβ1-40 in CSF, blood, or plasma, following 2, 13, 26, 52, 78, 104, 130, 156, 182, 208, 234, 260, 286, 312, 338, 332, 390, or 416 weeks of compound exposure. In one embodiment, the compound is used at a dose of between 5 and 10; 10 and 15; 15 and 20; 20 and 25; 25 and 30; 30 and 35; 35 and 40; 45 and 50; 50 and 55 mg; 55 and 60 mg; 60 and 100 mg; 100 and 200; 200 and 300 mg; 15 and 85 mg; 50 and 85 mg; 15 and 300 mg; or 50 and 300 mg per day. In one embodiment, the compound is used at a dose of between 10 and 30 mg per day. In one embodiment, the compound is used at a dose of between 30 and 50 mg per day. In one embodiment, the compound is used at a dose of 15 mg per day. In one embodiment, the compound is used at a dose of 50 mg per day. In one embodiment, the compound is used at a daily dose which results in a plasma steady state Cmax value of between 0 and 50; 50 and 100; 100 and 150; 150 and 200; 200 and 250; 250 and 300; 300 and 350; 350 and 400; 400 and 450; 450 and 500; 500 and 550; 550 and 600; 600 and 650; or 650 and 700 ng/ml. In one embodiment, the compound is used at a daily dose which results in a plasma steady state Cmax value of between 70 and 170 ng/ml. In one embodiment, the compound is used at a daily dose which results in a plasma steady state Cmax value of between 200 and 500 ng/ml. In a further embodiment, the compound is used in free form.

In a further invention, there is provided the compound N-(6-((3R,6R)-5-amino-3,6-dimethyl-6-(trifluoromethyl)-3,6-dihydro-2H-1,4-oxazin-3-yl)-5-fluoropyrid in-2-yl)-3-chloro-5-(trifluoromethyl)picolinamide, or a pharmaceutically acceptable salt thereof, for use as a medicament, wherein the patient treated with the medicament is not simultaneously treated with an inhibitor or inducer of CYP3A4. In another aspect of the further invention, there is provided the compound N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, or a pharmaceutically acceptable salt thereof, for use in the treatment or prevention of Alzheimer's disease, wherein the patient is not simultaneously treated with an inhibitor or inducer of CYP3A4. In one embodiment of this further invention, the patient is not simultaneously treated with an inhibitor or inducer of CYP3A4 for a period longer than three months. In one embodiment of this further invention, the patient is simultaneously treated with a CYP3A4 inhibitor or inducer for a period no longer than three months. In a further embodiment, the CYP3A4 inhibitor is a strong, moderate, or weak inhibitor of CYP3A4; and the CYP3A4 inducer is a strong, moderate, or weak inducer of CYP3A4. In a further embodiment, the CYP3A4 inhibitor is a strong inhibitor of CYP3A4; and the CYP3A4 inducer is a strong inducer of CYP3A4. In a further embodiment, the compound is used at a dose of 15 or 50 mg per day. In a further embodiment, the compound is used in free form. In another embodiment, the compound is comprised within a pharmaceutical composition.

### Definitions

As used herein, the term “Compound 1” or “Cmpd 1” refers to N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide and having the following structural formula:

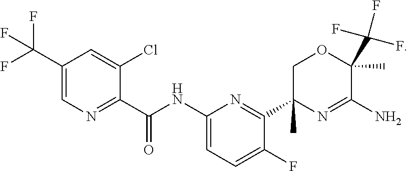 [SMILES: C[C@@]1(c2nc(NC(=O)c3ncc(C(F)(F)F)cc3Cl)ccc2F)CO[C@@](C)(C(F)(F)F)C(N)=N1]

In Example 1, using an alternative chemical naming format, “Compound 1” is also referred to as 3-chloro-5-trifluoromethyl-pyridine-2-carboxylic acid [6-((3R,6R)-5-amino-3,6-dimethyl-6-trifluoromethyl-3,6-dihydro-2H-[1,4]oxazin-3-yl)-5-fluoro-pyridin-2-yl]-amide.

The terms “Compound 1”, “Cmpd 1” and its corresponding full chemical name are used interchangeably throughout the description of the invention. It is intended that the term refers to the compound in either free form or pharmaceutically acceptable salt form, unless the context clearly indicates that only one form of the compound is intended. Compound 1 is described in WO 2012/095469 A1, Example 34. WO 2012/095469 A1 is incorporated herewith by reference in its entirety, in particular the disclosure related to the synthesis of Example 34.

As used herein, the term “Alzheimer's disease” or “AD” encompasses both preclinical and clinical Alzheimer's disease unless the context makes clear that either only preclinical Alzheimer's disease or only clinical Alzheimer's disease is intended.

As used herein, the term “clinical Alzheimer's disease” or “clinical AD” encompasses both Mild Cognitive Impairment (MCI) due to AD and dementia due to AD, unless the context makes clear that either only MCI due to AD or dementia due to AD is intended.

As used herein, the term “preclinical Alzheimer's disease” or “preclinical AD” refers to the presence of in vivo molecular biomarkers of AD in the absence of clinical symptoms. The National Institute on Aging and Alzheimer's Association provide a scheme, shown in Table 1 below, which sets out the different stages of preclinical AD (Sperling et al., 2011).

TABLE 1
Preclinical AD staging categories

| | | | Markers of | Evidence of |
|---|---|---|---|---|
| | | | neuronal injury | subtle |
| | | Aβ (PET | (tau, FDG, | cognitive |
| Stage | Description | or CSF) | sMRI) | change |
| Stage 1 | Asymptomatic cerebral | Positive | Negative | Negative |
| | amyloidosis | | | |
| Stage 2 | Asymptomatic amyloidosis + | Positive | Positive | Negative |
| | “downstream” neurodegeneration | | | |
| Stage 3 | Amyloidosis + neuronal injury + | Positive | Positive | Positive |
| | subtle cognitive/behavioral | | | |
| | decline | | | |

sMRI = structural magnetic resonance imaging

As used herein, the term “prevention of Alzheimer's disease” refers to the prophylactic treatment of AD; or delaying the onset or progression of AD. For example, the onset or progression of AD is delayed for at least 0.5, 1, 2, 3, 4, 5, 6, 7, 8, 9, or 10 years. In one embodiment, “prevention of Alzheimer's disease” refers to the prophylactic treatment of preclinical AD; or delaying the onset or progression of preclinical AD. In a further embodiment, the onset or progression of preclinical AD is delayed for at least 0.5, 1, 2, 3, 4, 5, 6, 7, 8, 9, or 10 years. In another embodiment, “prevention of Alzheimer's disease” refers to the prophylactic treatment of clinical AD; or delaying the onset or progression of clinical AD. In a further embodiment, the onset or progression of clinical AD is delayed for at least 0.5, 1, 2, 3, 4, 5, 6, 7, 8, 9, or 10 years.

Delay in the onset or progression of preclinical AD may be assessed by measuring in vivo molecular biomarkers relative to an initial baseline value, for example, by measuring:
(a) a reduction in brain amyloid deposition. For example, by measuring a change from baseline in composite cortical amyloid standard uptake value ratio (SUVR) using positron emission tomography (PET) imaging. A suitable PET tracer for the measurement of SUVR ratios is ¹⁸F-florbetapir (((E)-4-(2-(6-(2-(2-(2-([¹⁸F]-fluoroethoxy)ethoxy)ethoxy)pyridin-3-yl)vinyl)-N-methyl benzenamine)). By this method, the development of amyloid accumulation over time in independent samples of non-demented individuals may be measured (Palmqvist S et al., 2015). SUVR measurements may be calculated in pre-defined cortical brain regions of interest (ROls) referenced to tracer uptake in a pre-defined reference region. Cortical ROIs include areas known to have high amyloid deposition in AD, including, but not limited to, the parietal, occipital, lateral temporal and mesial temporal neocortical regions, as well as regions typically affected in early AD (Vlassenko A G et al., 2012). In one embodiment, brain amyloid deposition relative to an initial baseline value is reduced to a rate of less than 0, 1, 2, 3, 4, 5, 6, 7, 8, 9 or 10.0% per year of treatment;
(b) an effect on the underlying tau pathology, more specifically using PET and a suitable Tau tracer, for example ¹⁸F-THK5351 (Harada R et al., 2016), to measure the SUVR change from baseline in brain Tau pathology or using cerebrospinal fluid (CSF) to measure total Tau and phosphorylated Tau (Forlenza O V et al., 2015). In one embodiment, the levels of CSF Tau or phosphorylated Tau are reduced relative to an initial baseline value by at least 5, 10, 15, 20, 25, 30, 35, 40, 45 or 50% per year of treatment;
(c) an effect on neuronal glucose metabolism, density and/or activity using ¹⁸F-FDG (2-deoxy-2-[¹⁸F]fluoroglucose) PET (200 MBq for each scan). The ¹⁸F-FDG PET signal in AD-affected brain regions has been shown to be associated with cognitive impairment, subsequent cognitive decline and neuropathology in AD and to progress over time in the clinical and preclinical stages of AD, and is a disease and treatment efficacy biomarker (Foster N L et al., 2007). Data is analysed to determine the change in glucose metabolism, relative to a selected reference region. In one embodiment, the decrease in neuronal glucose metabolism in an AD-affected brain region as determined by ¹⁸F-FDG PET relative to an initial baseline value is limited to less than 5, 10, 15, 20, 25 or 30% per year of treatment;
(d) a slower decline in brain volume loss, as assessed by volumetric magnetic resonance imaging (vMRI) to measure a change from baseline in brain volume. vMRI can be used to measure a change in hippocampus, lateral ventricle, and total brain volume. In one embodiment, hippocampus volume loss is limited to less than 1, 2, or 3% per year of treatment; or
(e) the CSF Aβ1-42/Aβ1-40 ratio over time, for example in subjects having a baseline CSF Aβ1-42/Aβ1-40 ratio below 0.09, indicative of cortical amyloid deposition, as described herein in Example 10. In one embodiment, CSF Aβ1-42/Aβ1-40 ratio increases relative to an initial baseline value by at least 10, 20, 30, 40, 50, 80, 100, 200% over a period of at least 3, 6, 9, 12, 18, 24, or 36 months.

Delay in the onset or progression of preclinical AD may also be assessed relative to an initial baseline value using a sensitive cognitive measure to track changes in the preclinical stages of the disease, for example, using the Alzheimer's Prevention Initiative (API) preclinical composite cognitive (APCC) test battery. The APCC was developed as a sensitive tool to detect and track cognitive decline in individuals at risk to progress to the clinical stages of late onset AD (LOAD) (Langbaum J B et al., 2014).

Delay in the onset of clinical AD may be assessed by measuring a delay in cognitive and functional impairment due to AD, for example, by measuring a delay in the time to clinical diagnosis of Mild Cognitive Impairment (MCI) due to AD and/or dementia due to AD. The core clinical diagnostic criteria proposed by the National Institute on Aging—Alzheimer's Association Working Group may, for example, be used for the diagnosis of MCI (Albert M S et al., 2011) or dementia (McKhann G M et al., 2011). The European Medicines Agency (EMA) in its “Draft guidelines on the clinical investigation of medicines for the treatment of AD and other dementias” (EMA/Committee for Medicinal Products for Human Use (CHMP)/539931/2014) summarises the National Institute on Aging criteria for the diagnosis of MCI due to AD and AD dementia as set out below.

Diagnosis of MCI due to AD requires evidence of intra-individual decline, manifested by:
a) A change in cognition from previously attained levels, as noted by self- or informant report and/or the judgment of a clinician.
b) Impaired cognition in at least one domain (but not necessarily episodic memory) relative to age- and education-matched normative values; impairment in more than one cognitive domain is permissible.
c) Preserved independence in functional abilities, although the criteria also accept ‘mild problems’ in performing instrumental activities of daily living (IADL) even when this is only with assistance (i.e. rather than insisting on independence, the criteria allow for mild dependence due to functional loss).
d) No dementia, which nominally is a function of c (above).
e) A clinical presentation consistent with the phenotype of AD in the absence of other potentially dementing disorders. Increased diagnostic confidence may be suggested by
   1) Optimal: A positive Aβ biomarker and a positive degeneration biomarker
   2) Less optimal:
      i. A positive Aβ biomarker without a degeneration biomarker
      ii. A positive degeneration biomarker without testing for Aβ biomarkers

Diagnosis of AD dementia requires:
a) The presence of dementia, as determined by intra-individual decline in cognition and function.
b) Insidious onset and progressive cognitive decline.
c) Impairment in two or more cognitive domains; although an amnestic presentation is most common, the criteria allow for diagnosis based on nonamnestic presentations (e.g. impairment in executive function and visuospatial abilities).
d) Absence of prominent features associated with other dementing disorders.
e) Increased diagnostic confidence may be suggested by the biomarker algorithm discussed in the MCI due to AD section above.

Cognitive impairment and decline in the diagnosis of MCI due to AD and AD dementia may be measured using a sensitive cognitive measure to track changes in the clinical stages of the disease, for example, using:
a) the Clinical Dementia Rating (CDR) Scale—Sum of Boxes (SOB). The CDR is a global measure that evaluates cognition and functional performance and is widely used in clinical research in AD (Morris J C, 1993). The scale assesses six domains: Memory, Orientation, Judgment & Problem Solving, Community Affairs, Home & Hobbies, and Personal Care. Each domain is assigned a score, which are summed to obtain the sum of boxes (SOB) score;
b) the Repeatable Battery for the Assessment of Neuropsychological Status (RBANS). The RBANS (Randolph C, 1998) is a clinical tool that was specifically designed for both diagnostic purposes and for tracking change in neurocognitive status over time. One of the key design goals of the battery is to detect and characterize very mild dementia; or c) the Everyday Cognition Scale (ECog). The ECog measures cognitively-relevant everyday abilities comprised of 39 items covering 6 cognitively-relevant domains: Everyday Memory, Everyday Language, Everyday Visuospatial Abilities, Everyday Planning, Everyday Organization, and Everyday Divided Attention (Farias S T et al., 2008).

Suitable Aβ biomarkers for use in the diagnosis of MCI due to AD and AD dementia include, for example, CSF Aβ1-40, Aβ1-42 or PET imaging of beta amyloid neuritic plaques in the brain, as described above.

Suitable degeneration biomarkers for use in the diagnosis of MCI due to AD and AD dementia are described above in relation to the in vivo molecular biomarkers used to assess delay in the onset or progression of preclinical AD and include, for example, an effect on the underlying tau pathology; an effect on neuronal glucose metabolism; or a slower decline in brain volume loss.

As used herein, the term “patient” refers to a human subject.

As used herein, the term “patient at risk of developing clinical symptoms of Alzheimer's disease” refers to:
(a) a human subject with a genetic predisposition for the development of the clinical symptoms of Alzheimer's disease, for example:
   i. subjects carrying mutations in the genes for amyloid precursor protein (APP) or presenilin-1 and -2 (O'Brien R J, Wong P C, 2011), or
   ii. subjects carrying one or two copies of the ApoE4 allele (Liu C C et al., 2013);
(b) a human subject with Down Syndrome (Head E et al., 2012); or
(c) a human subject over 84 years of age.

As used herein, the term “amyloid-positive” refers to a patient who has detectable levels of accumulated Aβ in the brain. In one embodiment, a patient is “amyloid-positive” if the patient has detectable levels of accumulated Aβ in the brain based on an assessment of Aβ in the CSF or amyloid PET imaging, or both. As used herein, the term “amyloid-positivity determined by PET” refers to an increased level of amyloid PET tracer retention compared to background. Suitable PET tracers for the measurement of amyloid-positivity include ¹⁸F-florbetapir (Palmqvist S et al., 2015), ¹⁸F-florbetaben (NeuraCeq) and ¹⁸F-flutemetamol (Vizamyl). For example, an SUVR of 1.1 or higher on a brain ¹⁸F-florbetapir PET scan (260 MBq for each scan) may be used as an amyloid-positivity diagnostic threshold (Schreiber S et al., 2015). An SUVR of 1.2 or 1.3 could also be used as a threshold value.

As used herein, the term “amyloid-positivity determined by CSF measurement” refers to a reduced CSF Aβ1-42 value compared to that observed in a healthy control group. For example, amyloid-positivity may be determined by an Aβ1-42 value of 192 ng/L or less in CSF (Mattsson N et al., 2015). However, the CSF Aβ1-42 cut-off value used to determine amyloid-positivity will vary depending on the particular technique used (Forlenza O V et al., 2015). Amyloid positivity may also be determined by an Aβ1-42/Aβ1-40 ratio of less than 0.09 in CSF (Janelidze S et al., 2016). In one embodiment, the Aβ1-42/Aβ1-40 or Aβ42/Aβ40 ratio is less than 0.20, 0.15, 0.10, 0.09, 0.08, 0.07, 0.06 or 0.05 or between 0.20 and 0.01, 0.15 and 0.01, 0.10 and 0.01, or 0.05 and 0.01. Aβ1-40 and Aβ1-42 values may be measured using standard immunoassay techniques, for example using a monoclonal single antibody sandwich enzyme-linked immunosorbent (ELISA) assay on the Luminex platform (Herskovitz A Z et al., 2013) or the Meso Scale Discovery (MSD) 96-well MULTI-ARRAY human/rodent (6E10) Aβ40 and 42 sandwich immunoassays (Meso Scale Discovery, Rockville, Md., USA).

As used herein, the term “CYP3A4” refers to Cytochrome P450 3A4. CYP3A4 is an enzyme which plays a major role in the metabolism of a large variety of drugs (Luo G et al., 2004).

As used herein, the term “inducer of CYP3A4” refers to a drug which causes CYP3A4 activity levels to increase. Examples of CYP3A4 inducers include, but are not limited to, carbamazepine, phenytoin, rifampicin, and St John's wort. Techniques suitable for the measurement of CYP3A4 activity are well known (see, for example, Sevrioukova I F and Poulos T L, 2015). “Strong”, “moderate”, and “weak” inducers of CYP3A4 are drugs that decrease the plasma area under the curve (AUC) of Compound 1 (calculated as the area under the curve from 0 to infinity (AUCinf)) by ≧80%, ≧50% to <80%, and ≧20% to <50%, respectively. In one embodiment, the “inducer of CYP3A4” is a “strong inducer of CYP3A4.” Examples of strong inducers of CYP3A include, but are not limited to, carbamazepine, enzalutamide, mitotane, phenytoin, rifampin (also known as rifampicin), and St. John's wort. Examples of moderate inducers of CYP3A include, but are not limited to, bosentan, efavirenz, etravirine, and modafinil, Examples of weak inducers of CYP3A include, but are not limited to, armodafinil and rufinamide. See http://www.fda.gov/Drugs/DevelopmentApprovalProcess/DevelopmentResources/Drug InteractionsLabeling/ucm093664.htm#table3-3 (last visited Oct. 11, 2016).

As used herein, the term “inhibitor of CYP3A4” refers to a drug which causes CYP3A4 activity levels to decrease. Techniques suitable for the measurement of CYP3A4 activity are well known (see, for example, Sevrioukova I F and Poulos T L, 2015). Examples of CYP3A4 inhibitors include, but are not limited to, clarithromycin, grapefruit juice, and itraconazole. “Strong”, “moderate”, and “weak” inhibitors of CYP3A4 are drugs that increase the plasma AUC of Compound 1 (calculated as the area under the curve from 0 to infinity (AUCinf))≧5-fold, ≧2 to <5-fold, and ≧1.25 to <2-fold, respectively. In one embodiment the “inhibitor of CYP3A4” is a “strong inhibitor of CYP3A4.” Examples of strong inhibitors of CYP3A include, but are not limited to, boceprevir, cobicistat, conivaptan, danoprevir and ritonavir, elvitegravir and ritonavir, grapefruit juice, indinavir and ritonavir, itraconazole, ketoconazole, lopinavir and ritonavir, paritaprevir and ritonavir and (ombitasvir and/or dasabuvir), posaconazole, ritonavir, saquinavir and ritonavir, telaprevir, tipranavir and ritonavir, troleandomycin, voriconazole, clarithromycin, diltiazem, idelalisib, nefazodone, and nelfinavir. Examples of moderate inhibitors of CYP3A include, but are not limited to, aprepitant, cimetidine, ciprofloxacin, clotrimazole, crizotinib, cyclosporine, dronedarone, erythromycin, fluconazole, fluvoxamine, imatinib, tofisopam, and verapamil. Examples of weak inhibitors of CYP3A include, but are not limited to, chlorzoxazone, cilostazol, fosaprepitant, istradefylline, ivacaftor, lomitapide, ranitidine, ranolazine, tacrolimus, and ticagrelor. See http://www.fda.gov/Drugs/DevelopmentApprovalProcess/DevelopmentResources/DrugInteractionsLabeling/ucm093664.htm#table3-2 (last visited Oct. 11, 2016).

As used herein, term “simultaneously treated with an inhibitor or inducer of CYP3A4” refers to a situation where a patient is subjected to a therapeutic regimen with an inhibitor or inducer of CYP3A4 while also subjected to a therapeutic regimen with Compound 1. In one embodiment, the patient is not simultaneously treated with an inhibitor or inducer of CYP3A4 and Compound 1 for longer than 1, 2, 3, 4, 5, 6, 7, 8, 9, 10, 11, 12, 13, 14, 15 or 16 weeks. In another embodiment, the patient is not simultaneously treated with an inhibitor or inducer of CYP3A4 and Compound 1 for longer than 1, 2, 3, 4, 5, 7, 10, or 12 months. In a certain embodiment, the patient is not simultaneously treated with an inhibitor or inducer of CYP3A4 and Compound 1 for longer than 3 months.

As used herein, the term “pharmaceutically acceptable salt” refers to salts that retain the biological effectiveness of the compound of this invention and which typically are not biologically or otherwise undesirable (Pharmaceutical Salts: Properties, Selection, and Use, 2^{nd} Revised Edition (2011) P. Heinrich Stahl, Camille G. Wermuth).

As used herein, a “pharmaceutical composition” comprises the compound of this invention, or a pharmaceutically acceptable salt thereof, and at least one pharmaceutically acceptable carrier, in a solid form (typically a gelatin capsule) suitable for oral administration. An example of a pharmaceutical composition suitable for use in the prevention of AD in patients at risk of developing clinical symptoms of AD, and its method of preparation, is provided herein in Example 11.

The term “a therapeutically effective amount” of a compound of the present invention refers to an amount of the compound of the present invention that will elicit inhibition of BACE-1 in a patient as evidenced by a reduction in CSF or plasma Aβ1-40 levels relative to an initial baseline value.

For clarification, whenever a range is provided herein, said range is meant to include the endpoints. For example, a dose range between 30 and 50 mg per day comprises also doses of 30 and 50 mg per day.

### List of Abbreviations

| Abbreviation | Description |
|---|---|
| APP | amyloid precursor protein |
| Aβ | beta-amyloid peptide |
| aq. | aqueous |
| AUC | area under the curve, used to describe compound exposure |
| AUEC | area under the effect curve, used to describe effect over |
| | time |
| Aβ40 | beta-amyloid peptide 40 |
| Aβ42 | beta-amyloid peptide 42 |
| Aβ 1-40 | beta-amyloid peptide 1-40 |
| Aβ 1-42 | beta-amyloid peptide 1-42 |
| BACE-1 | beta site APP cleaving enzyme-1 |
| BACE-2 | beta site APP cleaving enzyme-2 |
| BACE | beta site APP cleaving enzyme |
| BLQ | below limit of quantification |
| Boc₂O | di-tert-butyl dicarbonate |
| C | concentration |
| CB | clinical biochemistry |
| Cb | cerebellum sample |
| CI | confidence interval |
| Comp. | compound |
| conc. | concentrated |
| Cpd | compound |
| CPP | Chemical and Pharmaceutical Profiling (department) |
| CSF | Cerebrospinal fluid |
| CVP | cava vena puncture blood sample |
| d | day |
| DCM | dichloromethane |
| DDI | drug-drug interaction |
| DEA | diethylamine |
| DMF | N,N-dimethylformamide |
| DMSO | dimethylsulfoxide |
| EDC | 1-(3-dimethylaminopropyl)-3-ethylcarbodiimide |
| EDTA | ethylenediamine tetraethyl acetate |
| ESI | electrospray ionisation |
| EtOAc | ethyl acetate |
| FA | formic acid |
| Fbr | forebrain sample |
| g | gram/gravitational acceleration |
| GFAP | Glial fibrillary acidic protein (astrocyte marker) |
| h, hr | hour |
| HOAt | 1-hydroxy-7-azabenzotriazole |
| HPLC, LC | high-performance liquid chromatography, liquid |
| | chromatography |
| IBA1 | Ionized calcium binding adaptor molecule 1 (microglia |
| | marker) |
| IC₅₀ | Inhibitory concentration 50 |
| kg | kilogram |
| LLOQ | Lower limit of quantification |
| LSmeans | least squares means |
| MeOH | methanol |
| min | minute(s) |
| ml | milliliter |
| μl | microliter |
| μM | micromolar |
| μmol | micromoles |
| MC | methylcellulose |
| min | minute |
| MS | mass spectrometry |
| MSD | MesoScale Discovery (Supplier of immunoassay kits) |
| NaCl | sodium chloride |
| nM | nanomolar |
| nmol | nanomoles |
| NMR | nuclear magnetic resonance spectrometry |
| ns | not significant |
| PD | pharmacodynamic |
| PET | positron emission tomography |
| pg | picogram |
| PK | pharmacokinetic |
| PMEL | premelanosome protein |
| pmol | picomoles |
| p.o. | per os |
| q.d. or QD | quaque die |
| q.s. | quam satis |
| Rel. | relative |
| Rf | retention factor |
| rpm | revolutions per minute |
| Rt | retention time (min) |
| RT, rt | room temperature |
| SEM | standard effort of the mean |
| SD | standard deviation |
| Stats | statistics |
| Swe | Swedish, indicating the presence of the “Swedish” double |
| | mutation in APP |
| t_{1/2} | half-life |
| TBME | tert-butyl-methyl-ether |
| THF | tetrahydrofuran |
| tiss | tissue |
| TLC | thin layer chromatography |
| Tris | tris-hydroxymethyl(aminomethane) buffer substance |
| TBS | tris-buffered saline |
| TX-100 | triton-X-100 (detergent, CAS No. 9002-93-1) |
| UPLC | ultra performance liquid chromatography |
| vs | versus |

### EXAMPLES

The following Examples illustrate how Compound 1 may be prepared (Example 1) and formulated (Example 11); demonstrate that Compound 1 is effective in reducing Aβ levels in wild type mice in the absence of an undesirable hair discolouration side effect observed with comparator compound NB-360 (Example 2); show the PK/PD effects of Compound 1 in an APOE4 transgenic mouse model (Example 3); show the PD effects of Compound 1 in a First in human clinical study (Example 4); demonstrate the safety and tolerability of Compound 1 in a 3-month clinical study (Example 5); show the effect of ApoE4 genotype on Compound 1 PD response in the 3-month clinical study (Example 6); demonstrate the therapeutic effectiveness of Compound 1 in reducing amyloid plaque number and area in the APP23 AD mouse model (Example 7); illustrate how a Compound efficacy study could be performed in ApoE4 homozygote at-risk patients (Example 8); show how the AUC of Compound 1 is affected when given in combination with a strong inhibitor or inducer of CYP3A4 (Example 9); and demonstrate how treatment with Compound 1 affects the underlying AD pathology in both ApoE4 carrier and non-carrier patients (Example 10).

### Example 1: Preparation of Compound 1

The preparation of Compound 1 is described in WO 2012/095469 A1 (Example 34). Compound 1 may also be prepared as described below.

### NMR Methodology

Proton spectra are recorded on a Bruker 400 MHz ultrashield spectrometer unless otherwise noted. Chemical shifts are reported in ppm relative to methanol (δ 3.31), dimethyl sulfoxide (δ 2.50), or chloroform (δ 7.29). A small amount of the dry sample (2-5 mg) is dissolved in an appropriate deuterated solvent (0.7 mL). The shimming is automated and the spectra obtained in accordance with procedures well known to the person of ordinary skill in the art.

### General Chromatography Information

HPLC method H1 (Rt_{H1}):
HPLC-column dimensions: 3.0×30 mm
HPLC-column type: Zorbax SB-C18, 1.8 μm
HPLC-eluent: A) water+0.05 Vol.-% TFA; B) ACN+0.05 Vol.-% TFA
HPLC-gradient: 30-100% B in 3.25 min, flow=0.7 ml/min

### LCMS Method H2 (RtH2):

HPLC-column dimensions: 3.0×30 mm
HPLC-column type: Zorbax SB-C18, 1.8 μm
HPLC-eluent: A) water+0.05 Vol.-% TFA, B) ACN+0.05 Vol.-% TFA
HPLC-gradient: 10-100% B in 3.25 min, flow=0.7 ml/min

### UPLCMS Method H3 (Rtₕ₃):

HPLC-column dimensions: 2.1×50 mm
HPLC-column type: Acquity UPLC HSS T3, 1.8 μm
HPLC-eluent: A) water+0.05 Vol.-% formic acid+3.75 mM ammonium acetate B) ACN+0.04 Vol.-% formic acid
HPLC-gradient: 2-98% B in 1.4 min, 98% B 0.75 min, flow=1.2 ml/min
HPLC-column temperature: 50° C.

### LCMS Method H4 (Rtₕ₄):

HPLC-column dimensions: 3.0×30 mm
HPLC-column type: Zorbax SB-C18, 1.8 μm
HPLC-eluent: A) water+0.05 Vol.-% TFA; B) ACN+0.05 Vol.-% TFA
HPLC-gradient: 70-100% B in 3.25 min, flow=0.7 ml/min

### LCMS Method H5 (RtH5):

HPLC-column dimensions: 3.0×30 mm
HPLC-column type: Zorbax SB-C18, 1.8 μm
HPLC-eluent: A) water+0.05 Vol.-% TFA; B) ACN+0.05 Vol.-% TFA
HPLC-gradient: 80-100% B in 3.25 min, flow=0.7 ml/min

### LCMS Method H6 (RtH6):

HPLC-column dimensions: 3.0×30 mm
HPLC-column type: Zorbax SB-C18, 1.8 μm
HPLC-eluent: A) water+0.05 Vol.-% TFA; B) ACN+0.05 Vol.-% TFA
HPLC-gradient: 40-100% B in 3.25 min, flow=0.7 ml/min

### a) 2-Bromo-5-fluoro-4-triethylsilanyl-pyridine

A solution of diisopropylamine (25.3 g, 250 mmol) in 370 ml THF was cooled with a dry-ice acetone bath at −75° C. BuLi (100 ml, 250 mmol, 2.5 M in hexanes) was added dropwise while maintaining the temperature below −50° C. After the temperature of the mixture had reached −75° C. again, a solution of 2-bromo-5-fluoropyridine (36.7 g, 208 mmol) in 45 ml THF was added dropwise. The mixture was stirred for 1 h at −75° C. Triethylchlorosilane (39.2 g, 260 mmol) was added quickly. The temperature stayed below −50° C. The cooling bath was removed and the reaction mixture was allowed to warm to −15° C., poured onto aq. NH₄Cl (10%). TBME was added and the layers were separated. The organic layer was washed with brine, dried with MgSO₄.H₂O, filtered and evaporated to give a brown liquid which was distilled at 0.5 mm Hg to yield the title compound as a slightly yellow liquid (b.p. 105-111° C.). HPLC: Rt_{H4}=2.284 min; ESIMS: 290, 292 [(M+H)₊, 1Br]; ¹H-NMR (400 MHz, CDCl₃): 8.14 (s, 1H), 7.40 (d, 1H), 1.00-0.82 (m, 15H).

### b) 1-(6-Bromo-3-fluoro-4-triethylsilanyl-pyridin-2-yl)-ethanone

A solution of diisopropylamine (25.4 g, 250 mmol) in 500 ml THF was cooled to −75° C. BuLi (100 ml, 250 mmol, 2.5 M in hexanes) was added dropwise while maintaining the temperature below −50° C. After the reaction temperature had reached −75° C. again, a solution of 2-bromo-5-fluoro-4-triethylsilanyl-pyridine (56.04 g, 193 mmol) in 60 ml THF was added dropwise. The mixture was stirred in a dry ice bath for 70 minutes. N,N-dimethylacetamide (21.87 g, 250 mmol) was added quickly, the reaction temperature rose to −57° C. The reaction mixture was stirred in a dry ice bath for 15 min and then allowed to warm to −40° C. It was poured on a mixture of 2M aq. HCl (250 ml, 500 mmol), 250 ml water and 100 ml brine. The mixture was extracted with TBME, washed with brine, dried over MgSO₄.H₂O, filtered and evaporated to give a yellow oil which was purified on a silica gel column by eluting with hexane/0-5% TBME to yield 58.5 g of the title compound as a yellow liquid. TLC (Hex/TBME 99/1): R_{f}=0.25; HPLC: Rt_{H4}=1.921 min; ESIMS: 332, 334 [(M+H)⁺, 1Br]; ¹H-NMR (400 MHz, CDCl₃): 7.57 (d, 1H), 2.68 (s, 3H), 1.00-0.84 (m, 15H).

### c) (S)-2-(6-Bromo-3-fluoro-4-triethylsilanyl-pyridin-2-yl)-2-trimethylsilanyloxy-propionitrile

At first, the catalyst solution was prepared by dissolving water (54 mg, 3.00 mmol) in 100 ml dry DCM 00.001% water). This wet DCM (44 ml, 1.32 mmol water content) was added to a well stirred solution of titanium(IV) butoxide (500 mg, 1.47 mmol) in 20 ml dry DCM. The resulting clear solution was refluxed for 1 h. This solution was then cooled to rt and 2,4-di-tert-butyl-6-{[(E)-(S)-1-hydroxymethyl-2-methyl-propylimino]-methyl}-phenol [CAS 155052-31-6] (469 mg, 1.47 mmol) was added. The resulting yellow solution was stirred at rt for 1 h. This catalyst solution (0.023 M, 46.6 ml, 1.07 mmol) was added to a solution of 1-(6-bromo-3-fluoro-4-triethylsilanyl-pyridin-2-yl)-ethanone (35.53 g, 107 mmol) and trimethylsilyl cyanide (12.73 g, 128 mmol) in 223 ml dry DCM. The mixture was stirred for 2 days and evaporated to give 47 g of the crude title compound as an orange oil. HPLC: Rt_{H5}=2.773 min; ESIMS: 431, 433 [(M+H)⁺, 1Br]; ¹H-NMR (400 MHz, CDCl₃): 7.46 (d, 1H), 2.04 (s, 3H), 1.00 (t, 9H), 1.03-0.87 (m, 15H), 0.20 (s, 9H).

### d) (R)-1-Amino-2-(6-bromo-3-fluoro-4-triethylsilanyl-pyridin-2-yl)-propan-2-ol hydrochloride

Borane dimethyl sulfide complex (16.55 g, 218 mmol) was added to a solution of crude (S)-2-(6-bromo-3-fluoro-4-triethylsilanyl-pyridin-2-yl)-2-trimethylsilanyloxy-propionitrile (47 g, 109 mmol) in 470 ml THF. The mixture was refluxed for 2 h. The heating bath was removed and the reaction mixture was quenched by careful and dropwise addition of MeOH. After the evolution of gas had ceased, aq. 6M HCl (23.6 ml, 142 mmol) was added slowly. The resulting solution was evaporated and the residue was dissolved in MeOH and evaporated (twice) to yield 44.5 g of a yellow foam, pure enough for further reactions. HPLC: Rt_{H1}=2.617 min; ESIMS: 363, 365 [(M+H)⁺, 1Br]; ¹H-NMR (400 MHz, CDCl₃): 7.93 (s, br, 3H), 7.53 (d, 1H), 6.11 (s, br, 1H), 3.36-3.27 (m, 1H), 3.18-3.09 (m, 1H), 1.53 (s, 3H), 0.99-0.81 (m, 15H).

### e) (R)—N-(2-(6-bromo-3-fluoro-4-(triethylsilyl)pyridin-2-yl)-2-hydroxypropyl)-4-nitrobenzenesulfonamide

To a solution of crude (R)-1-amino-2-(6-bromo-3-fluoro-4-triethylsilanyl-pyridin-2-yl)-propan-2-ol hydrochloride (43.5 g, 109 mmol) in 335 ml THF was added a solution of NaHCO₃ (21.02 g, 250 mmol) in 500 ml water. The mixture was cooled to 0-5° C. and a solution of 4-nitrobenzenesulfonyl chloride (26.5 g, 120 mmol) in 100 ml THF was added in a dropwise. The resulting emulsion was stirred overnight while allowing the temperature to reach rt. The mixture was extracted with TBME. The organic layer was dried with MgSO₄.H₂O, filtered and evaporated to give an orange resin which was purified on a silca gel column by eluting with Hexanes/10-20% EtOAc to yield 37.56 g of the title compound as a yellow resin. TLC (Hex/EtOAc 3/1): R_{f}=0.34; HPLC: Rt_{H4}=1.678 min; ESIMS: 548, 550 [(M+H)⁺, 1Br]; ¹H-NMR (400 MHz, DMSO-d₆): 8.40 (d, 2H), 8.06 (t, 1H), 7.97 (d, 2H), 7.45 (d, 1H), 5.42 (s, 1H), 3.23 (d, 2H), 1.44 (s, 3H) 0.97-0.81 (m, 15H); Chiral HPLC (Chiralpak AD-H 1213, UV 210 nm): 90% ee.

### f) 6-Bromo-3-fluoro-2-[(S)-2-methyl-1-(4-nitro-benzenesulfonyl)-aziridin-2-yl]-4-triethylsilanyl-pyridine

A solution of triphenylphosphine (21.55 g, 82 mmol) and (R)—N-(2-(6-bromo-3-fluoro-4-(triethylsilyl)pyridin-2-yl)-2-hydroxypropyl)-4-nitrobenzenesulfonamide (37.56 g, 69 mmol) in 510 ml THF was cooled to 4° C. A solution of diethyl azodicarboxylate in toluene (40% by weight, 38.8 g, 89 mmol) was added in a dropwise while maintaining the temperature below 10° C. The cooling bath was removed and the reaction mixture was stirred at rt for 1 h. The reaction mixture was diluted with approx. 1000 ml toluene and THF was removed by evaporation at the rotavap. The resulting toluene solution of crude product was pre-purified on a silca gel column by eluting with hexanes/5-17% EtOAc. Purest fractions were combined, evaporated and crystallized from TBME/hexane to yield 29.2 g of the title compound as white crystals. HPLC: Rt_{H4}=2.546 min; ESIMS: 530, 532 [(M+H)⁺, 1Br]; ¹H-NMR (400 MHz, CDCl₃): 8.40 (d, 2H), 8.19 (d, 2H), 7.39 (d, 1H), 3.14 (s, 1H), 3.02 (s, 1H), 2.01 (s, 3H) 1.03-0.83 (m, 15H); α[D]−35.7° (c=0.97, DCM).

### g) 6-Bromo-3-fluoro-2-[(S)-2-methyl-1-(4-nitro-benzenesulfonyl)-aziridin-2-yl]-pyridine

Potassium fluoride (1.1 g, 18.85 mmol) was added to a solution of 6-bromo-3-fluoro-2-[(S)-2-methyl-1-(4-nitro-benzenesulfonyl)-aziridin-2-yl]-4-triethylsilanyl-pyridine (5 g, 9.43 mmol) and AcOH (1.13 g, 9.43 mmol) in 25 ml THF. DMF (35 ml) was added and the suspension was stirred for 1 h at rt. The reaction mixture was poured onto a mixture of sat. aq. NaHCO₃ and TBME. The layers were separated and washed with brine and TBME. The combined organic layers were dried over MgSO₄H₂O, filtered and evaporated to give a yellow oil which was crystallized from TBME/hexane to yield 3.45 g of the title compound as white crystals. HPLC: Rt_{H6}=2.612 min; ESIMS: 416, 418 [(M+H)⁺, 1Br]; ¹H-NMR (400 MHz, CDCl₃): 8.41 (d, 2H), 8.19 (d, 2H), 7.48 (dd, 1H), 7.35 (t, 1H), 3.14 (s, 1H), 3.03 (s, 1H), 2.04 (s, 3H); α[D]−35.7° (c=0.89, DCM).

### h) (R)-2-[(R)-2-(6-Bromo-3-fluoro-pyridin-2-yl)-2-(4-nitro-benzenesulfonylamino)-propoxy]-3,3,3-trifluoro-2-methyl-propionic acid ethyl ester

A solution of (R)-3,3,3-trifluoro-2-hydroxy-2-methyl-propionic acid ethyl ester (11.93 g, 64.1 mmol) in DMF (158 ml) was evacuated/flushed with nitrogen twice. A solution of KOtBu (6.21 g, 55.5 mmol) in DMF (17 ml) was added dropwise while maintaining a reaction temperature of ca 25° C. using cooling with a water bath. After 15 min solid 6-bromo-3-fluoro-2-[(S)-2-methyl-1-(4-nitro-benzenesulfonyl)-aziridin-2-yl]-pyridine (17.78 g, 42.7 mmol) was added and stirring was continued for 3 h. The reaction mixture was poured onto a mixture of 1M HCl (56 ml), brine and TBME. The layers were separated, washed with brine and TBME. The combined organic layers were dried over MgSO₄H₂O, filtered and evaporated. The crude reaction product was purified via chromatography on silica gel (hexanes/25-33% TBME) to yield 16.93 g of the title compound as a yellow resin that was contaminated with an isomeric side-product (ratio 70:30 by ¹H-NMR).

HPLC: Rt_{H6}=2.380 min; ESIMS: 602, 604 [(M+H)⁺, 1Br]; ¹H-NMR (400 MHz, CDCl₃): 8.32 (d, 2H), 8.07 (d, 2H), 7.46-7.41 (m, 1H), 7.30-7.23 (m, 1H), 6.92 (s, 1H), 3.39-4.30 (m, 2H), 3.95 (d, 1H), 3.84 (d, 1H), 1.68 (s, 3H), 1.56 (s, 3H), 1.40-1.34 (m, 3H)+isomeric side-product.

### i) (R)-2-[(R)-2-(6-Bromo-3-fluoro-pyridin-2-yl)-2-(4-nitro-benzenesulfonylamino)-propoxy]-3,3,3-trifluoro-2-methyl-propionamide

A solution of (R)-2-[(R)-2-(6-bromo-3-fluoro-pyridin-2-yl)-2-(4-nitro-benzenesulfonylamino)-propoxy]-3,3,3-trifluoro-2-methyl-propionic acid ethyl ester (16.93 g, 28.1 mmol) in a NH₃/MeOH (7M, 482 ml) was stirred at 50° C. in a sealed vessel for 26 h. The reaction mixture was evaporated and the residue was crystallized from DCM to yield 9.11 g of the title compound as colorless crystals.

HPLC: Rt_{H6}=2.422 min; ESIMS: 573, 575 [(M+H)⁺, 1Br]; ¹H-NMR (400 MHz, CDCl₃): 8.33 (d, 2H), 8.06 (d, 2H), 7.42 (dd, 1H), 7.30-7.26 (m, 1H), 7.17 (s, br, 1H), 6.41 (s, 1H), 5.57 (s, br, 1H), 4.15 (m, 2H), 1.68 (s, 3H), 1.65 (s, 3H).

### j) N—[(R)-1-(6-Bromo-3-fluoro-pyridin-2-yl)-2-((R)-1-cyano-2,2,2-trifluoro-1-methyl-ethoxy)-1-methyl-ethyl]-4-nitro-benzenesulfonamide

A suspension of (R)-2-[(R)-2-(6-bromo-3-fluoro-pyridin-2-yl)-2-(4-nitro-benzenesulfonylamino)-propoxy]-3,3,3-trifluoro-2-methyl-propionamide (8.43 g, 14.70 mmol) and triethylamine (5.12 ml, 36.8 mmol) in 85 ml DCM was cooled to 0-5° C. Trifluoroacetic anhydride (2.49 ml, 17.64 mmol) was added dropwise over 30 min. Additional triethylamine (1.54 ml, 11.07 mmol) and trifluoroacetic anhydride (0.75 ml, 5.29 mmol) were added to complete the reaction. The reaction mixture was quenched by addition of 14 ml aqueous ammonia (25%) and 14 ml water. The emulsion was stirred for 15 min, more water and DCM were added and the layers were separated. The organic layer was dried with MgSO₄H₂O, filtered and evaporated. Purification by column chromatography on a silica gel (hexanes/10-25% EtOAc) gave 8.09 g of the title compound as a yellow resin.

HPLC: Rt_{H6}=3.120 min; ESIMS: 555, 557 [(M+H)⁺, 1Br]; ¹H-NMR (400 MHz, CDCl₃): 8.35 (d, 2H), 8.11 (d, 2H), 7.50 (dd, 1H), 7.32 (dd, 1H), 6.78 (s, 1H), 4.39 (d 1H), 4.22 (d, 1H), 1.68 (s, 6H).

### k) (2R,5R)-5-(6-Bromo-3-fluoro-pyridin-2-yl)-2,5-dimethyl-2-trifluoromethyl-5,6-dihydro-2H-[1,4]oxazin-3-ylamine

A solution of N—[(R)-1-(6-bromo-3-fluoro-pyridin-2-yl)-2-((R)-1-cyano-2,2,2-trifluoro-1-methyl-ethoxy)-1-methyl-ethyl]-4-nitro-benzenesulfonamide (9.18 g, 16.53 mmol) and N-acetylcysteine (5.40 g, 33.10 mmol) in 92 ml ethanol was evacuated and flushed with nitrogen. K₂CO₃ (4.57 g, 33.1 mmol) was added and the mixture was stirred at 80° C. for 3 days. The reaction mixture was concentrated in vacuo to about ¼ of the original volume and partitioned between water and TBME. The organic layer was washed with 10% aq. K₂CO₃ solution, dried over Na₂SO₄, filtered and evaporated to give a yellow oil. Column chromatography on silica (hexanes/14-50% (EtOAc:MeOH 95:5)) gave 4.55 g of the title compound as an off-white solid. HPLC: Rt_{H2}=2.741 min; ESIMS: 370, 372 [(M+H)⁺, 1Br]; ¹H-NMR (400 MHz, DMSO-d₆): 7.71-7.62 (m, 2H), 5.97 (s, br, 2H), 4.02 (d 1H), 3.70 (d, 1H), 1.51 (s, 3H), 1.47 (s, 3H).

### l) (2R,5R)-5-(6-Amino-3-fluoro-pyridin-2-yl)-2,5-dimethyl-2-trifluoromethyl-5,6-dihydro-2H-[1,4]oxazin-3-yl amine

A glass/stainless steel autoclave was purged with nitrogen, Cu₂O (0.464 g, 3.24 mmol), ammonia (101 ml, 25%, aq., 648 mmol, 30 equivalents) and (2R,5R)-5-(6-Bromo-3-fluoro-pyridin-2-yl)-2,5-dimethyl-2-trifluoromethyl-5,6-dihydro-2H-[1,4]oxazin-3-ylamine (8 g, 21.6 mmol) in ethylene glycol (130 ml) was added. The autoclave was closed and the suspension heated up to 60° C. and the solution was stirred for about 48 hours (max. pressure 0.7 bar, inside temperature 59-60° C.). The reaction mixture was diluted with ethyl acetate and water. The organic phase was washed with water and 4 times with 12% aq. ammonia and finally with brine, dried over sodium sulfate, filtered and evaporated. The crude product (7 g, containing some ethylen glycol, quantitative yield) was used in the next step without further purification. HPLC: Rt_{H3}=0.60 min; ESIMS: 307 [(M+H)⁺].

### m) [(2R,5R)-5-(6-Amino-3-fluoro-pyridin-2-yl)-2,5-dimethyl-2-trifluoromethyl-5,6-dihydro-2H-[1,4]oxazin-3-yl]-carbamic acid tert-butyl ester

A solution of (2R,5R)-5-(6-amino-3-fluoro-pyridin-2-yl)-2,5-dimethyl-2-trifluoromethyl-5,6-dihydro-2H-[1,4]oxazin-3-yl amine (6.62 g, 21.6 mmol), Boc₂O (4.72 g, 21.6 mmol) and Hünig's base (5.66 ml, 32.4 mmol) in dichloromethane (185 ml) was stirred at rt for 18 hours. The reaction mixture was washed with sat. aq. NaHCO₃ and brine. The aqueous layers were back extracted with dichloromethane and the combined organic layers were dried over sodium sulfate, filtered and evaporated to give a light green solid (14 g). The crude product was chromatographed over silicagel (cyclohexane:ethyl acetate 95:5 to 60:40) to afford 7.68 g of the title compound.

TLC (cyclohexane:ethyl acetate 3:1): R_{f}=0.21; HPLC: Rt_{H3}=1.14 min; ESIMS: 408 [(M+H)⁺]; ¹H-NMR (400 MHz, CDCl₃): 11.47 (br. s, 1H), 7.23 (dd, J=10.42, 8.78 Hz, 1H), 6.45 (dd, J=8.78, 2.64 Hz, 1H), 4.50 (br. s, 2H), 4.32 (d, J=2.38 Hz, 1H), 4.10 (d, J=11.80 Hz, 1H), 1.69 (s, 3H, CH3), 1.65 (s, 3H, CH3), 1.55 (s, 9H).

### n) ((2R,5R)-5-{6-[(3-Chloro-5-trifluoromethyl-pyridine-2-carbonyl)-amino]-3-fluoro-pyridin-2-yl}-2,5-dimethyl-2-trifluoromethyl-5,6-dihydro-2H-[1,4]oxazin-3-yl)-carbamic acid tert-butyl ester

A mixture of [(2R,5R)-5-(6-amino-3-fluoro-pyridin-2-yl)-2,5-dimethyl-2-trifluoromethyl-5,6-dihydro-2H-[1,4]oxazin-3-yl]-carbamic acid tert-butyl ester (3.3 g, 8.12 mmol), 3-chloro-5-trifluoromethylpicolinic acid (2.2 g, 9.74 mmol), HOAt (1.99 g, 14.62 mmol) and EDC hydrochloride (2.33 g, 12.18 mmol) was stirred in DMF (81 ml) at rt for 48 hours. The reaction mixture was diluted with ethyl acetate and washed with water and brine, dried over sodium sulfate, filtered and evaporated. The crude product (12 g) was chromatographed over silicagel (cyclohexane to cyclohexane:ethyl acetate 1:1) to yield 5.2 g of the title compound.

TLC (silica, cyclohexane:ethyl acetate 3:1): R_{f}=0.47; HPLC: Rt_{H3}=1.40 min; ESIMS: 615, 616 [(M+H)⁺, 1Cl]; ¹H-NMR (400 MHz, CDCl₃): 11.68 (s, 1H), 10.41 (s, 1H), 8.81 (dd, J=1.82, 0.69 Hz, 1H), 8.45 (dd, J=8.91, 3.14 Hz, 1H), 8.19 (dd, J=1.88, 0.63 Hz, 1H), 7.59 (dd, J=9.79, 9.16 Hz, 1H), 4.38 (d, J=2.13 Hz, 1H), 4.18 (d, J=11.80 Hz, 1H), 1.75 (s, 3H), 1.62 (s, 3H), 1.60 (s, 9H).

### o) 3-Chloro-5-trifluoromethyl-pyridine-2-carboxylic acid [6-((3R,6R)-5-amino-3,6-dimethyl-6-trifluoromethyl-3,6-dihydro-2H-[1,4]oxazin-3-yl)-5-fluoro-pyridin-2-yl]-amide

A mixture of ((2R,5R)-5-{6-[3-chloro-5-trifluoromethyl-pyridine-2-carbonyl)-amino]-3-fluoro-pyridin-2-yl}-2,5-dimethyl-2-trifluoromethyl-5,6-dihydro-2H-[1,4]oxazin-3-yl)-carbamic acid tert-butyl ester (4.99 g, 8.13 mmol) and TFA (6.26 ml, 81 mmol) in dichloromethane (81 ml) was stirred at rt for 18 hours. The solvent was evaporated and the residue diluted with a suitabable organic solvent, such as ethyl acetate and aq. ammonia. Ice was added and the organic phase was washed with water and brine, dried over sodium sulfate, filtered and evaporated to yield 3.78 g of the title compound.

HPLC: Rt_{H3}=0.87 min; ESIMS: 514, 516 [(M+H)⁺, 1Cl]; ¹H-NMR (400 MHz, DMSO-d₆): δ 11.11 (s, 1H), 9.06 (s, 1H), 8.69 (s, 1H), 8.13 (dd, J=8.8, 2.6 Hz, 1H), 7.80-7.68 (m, 1H), 5.88 (br. s, 2H), 4.12 (d, J=11.5 Hz, 1H), 3.72 (d, J=11.4 Hz, 1H), 1.51 (s, 3H), 1.49 (s, 3H).

### Example 2: Chronic Dosing of Compound 1 and Comparator Compound NB-360 in Wild-Type Mice

The studies described herein were carried out in commercial wildtype mice in order to investigate the chronic treatment effects of Compound 1, especially on discoloration of the fur, to determine an efficacious dose in wildtype mice, and to compare the window between efficacy and fur colour changes with that of comparator BACE-1 inhibitor compound NB-360 (N-(3-((3R,6R)-5-amino-3,6-dimethyl-6-(trifluoromethyl)-3,6-dihydro-2H-1,4-oxazin-3-yl)-4-fluorophenyl)-5-cyano-3-methylpicolinamide) (Neumann U et al., 2015; and Shimshek D R et al., 2016).

### Animals

C57BL/6 mice were ordered at Charles River Laboratories, France.

### Compound Formulation and Dosing

Compound 1 and NB-360 were formulated as a suspension. Vehicle, Compound 1 or NB-360 were given per os in a volume of 10 ml/kg once daily (mornings) for 8 weeks. Vehicle: 0.1 Tween80 in 0.5% methylcellulose in water.

### Body Weight and Fur Colour Scoring

Body weight was taken 3 times per week (Monday, Wednesday, Friday). Subjective scoring of any hair colour changes was performed once weekly (Wednesday). Scores (% of body with grey fur): 0: No change; 1: Spots; 2: >30%; 3: >50%; 4:>75%; 5: 100%. Animals were photo-documented when fur color change was observed. Final fur color scoring was performed blinded and by a person not involved in the study.

### Ex-Vivo Samples and Sample Harvest Methodology

Blood samples were used to analyze whole-blood compound levels and were obtained either from tail-vein during the in-life part into EDTA tubes (CB300, Sarstedt, Germany) or from trunk blood at the day of necropsy into EDTA Eppendorf tubes (Milian S A, CatNoTOM-14, Fisher Scientific, Wohlen, Switzerland), or into serum tubes (CB300Z, Sarstedt, Nümbrecht, Germany).

Plasma for amyloid-β (Aβ) analysis was collected by centrifugation of EDTA blood (8000 rpm/6800×g, 15 min, 4° C.) and collected into protein Lo-Bind Eppendorf tubes (003 0108.116, Eppendorf, Hamburg, Germany).

After 20 min at room temperature, serum was separated by centrifugation (8000×g, 15 min, 4° C.) and collected into protein Lo-Bind Eppendorf tubes to check for kidney toxicity biomarkers. All blood/plasma/serum samples were frozen on dry ice and stored at −80° C. until analysis.

Brain was removed immediately after decapitation, rinsed with saline and sectioned sagitally down the midline. The left half of the cerebellum was used to analyze compound level and was placed into a glass tube (Chromacol, 125×5-SV T051, Welwyn Garden City, United Kingdom), weighed and frozen in dry-ice, the left half of the forebrain (without olfactory bulb) was used for Aβ analysis, and was frozen on a metal plate on dry ice and placed into protein Lo-bind tube (003 0108.116, Eppendorf, Hamburg, Germany).

Ventral and dorsal skin were taken to analyze compound level, weighed and frozen on dry-ice.

### Analysis of Compound Levels

Compound 1 and NB-360 levels in biological samples were quantified in blood, brain and skin by liquid chromatography/tandem mass spectrometry (HPLC/MS/MS). Brain samples were mixed with 2 volumes of KH₂PO₄ buffer and homogenized using the Covaris® device. Skin samples were mixed with approx. 6-fold volumes of methanol/water and homogenized using a Precellys tube. Either 30 μL of blood, brain or skin homogenate were spiked with a structurally related internal standard and subsequently mixed with an at least 6-fold excess volume acetonitrile for protein precipitation. The supernatant was injected directly into the LC/MS/MS system for analysis.

TABLE 2
Instrumental conditions for blood and brain samples

| | |
|---|---|
| Analytical method | HPLC/MS/MS |
| MS | Sciex QTrap 5500; |
| | Heated Electrospray Ionisation in positive ion mode. |
| MS/MS methods | [CHS360]: |
| | 450.0 Da [M + H]⁺ → 292.0 Da/CE 37 eV |
| | Compound 1: |
| | 514.0 Da [M + H]⁺ 140.1/180.1 Da/CE 53/85 eV |
| | Internal standard (IS): |
| | 400.0 Da [M + H]⁺ → 131.0 Da/55 40 eV |
| HPLC | Flux Rheos Allegro (Thermo Scientific/Reinach BL, |
| | Switzerland) |
| HPLC columns | Phenomenex Kinetix C8 50 * 2.1 mm, 2.6 uM |
| | (Phenomenex, Torrance, CA, U.S.A.) |
| Buffer A // Buffer C | A: H₂O + 0.1% formic acid // C: acetonitrile + 0.1% |
| | formic acid |
| Gradients | Pump 1: 0.0′ 98% A, 2% C // 0.3′ 70% A, 30% C // |
| | 7.5′ 40% A, 60% C // 9.5′ 1% A, 99% C // |
| | 10.25′ 1% A, 99% C // 10.3′ 99% A, 1% C // |
| | 12.3′ 99% A, 1% C. Flow 300-350 μl/min |

TABLE 3
Instrumental conditions for skin samples

| | |
|---|---|
| Analytical method | HPLC/MS/MS |
| MS | TSQ Quantum ultra (Thermo Scientific, Waltham, MA, U.S.A.); |
| | Heated Electrospray Ionisation in positive ion mode. |
| MS/MS methods | [CHS360]: |
| | 450.0 Da [M + H]⁺ → 116.9 Da/CE 43 eV |
| | Compound 1: |
| | 514.0 Da [M + H]⁺ → 375.0 Da/CE 28 eV |
| | Internal standard (IS) |
| | 400.0 Da [M + H]⁺ → 131.0 Da/CE 40 eV |
| HPLC | Flux Rheos Allegro (Thermo Scientific/Reinach BL, Switzerland) |
| HPLC columns | Synergie Hydro RP 50 * 2.0 mm, 2.5 uM (Phenomenex, Torrance, |
| | CA, U.S.A.) |
| Buffer C // Buffer D | C: H₂O + 0.1% formic acid // D: Methanol + 0.1% formic acid |
| Gradients | Pump 1: 0.0′ 90% C, 10% D // 0.1′ 90% C, 10% D // 4.5′ 20% C, |
| | 80% D // 5.5′ 2% C, 98% D // 5.6′ 2% C, 98% D // 5.7′ 90% C, |
| | 10% D // 7.0′ 90% C, 10% D. Flow 400 μl/min |
| Acceptance criteria | Calibration standards: Bias within the range ±20% at the LLOQ |
| in each run | and at ±15% at the other concentration levels. At least ¾ of the |
| | individual back-calculated values with at least one value at both |
| | extremes of the standard curve fulfilling the acceptance criteria. |
| | Quality Control samples: Bias within the range ±30% for at least |
| | ⅔ of the individual values. At least one value at each QC level |
| | fulfilling the acceptance criteria. |
| | Dynamic range: 0.4 to 12500 ng/mL for all compounds |

### Analysis of Aβ40 in Mouse Brain

### Brain Homogenization

Frozen mouse forebrains were weighed and homogenized in 9 volumes (w/v) of ice-cold TBS-Complete (20 mM Tris-HCl pH 7.4, 137 mM NaCl, 1× Complete [Protease Inhibitor Cocktail Tablets: 1 836 145, Roche Diagnostics GmbH, Penzberg, Germany]) by sonication (90% duty cycle, output control 5, 40-55 pulses, [Sonifier 450, Branson]). After homogenization several 50 μl aliquots were prepared for analysis and were stored at −80° C.

### Preparation of Synthetic Aβ1-40 Solutions as Standards

Human Aβ peptide (1-40) trifluoroacetate salt (H 1194.1000, Bachem, Bubendorf, Switzerland) was used as calibration curve for Aβ1-40. It was solubilized in water-free DMSO (41647, Fluka) at a concentration of 1 mg/ml for approximately 30 min at room temperature (RT) and then visually checked for complete solubilization.

20×5 μl aliquots and 100 μl aliquots of the remaining solution were prepared in LoBind tubes (0030 108.094, Eppendorf, Hamburg, Germany), overlaid with nitrogen gas in order to protect the Aβ peptide from oxidation and stored at −80° C. For the calibration curves a 5 μl aliquot was used just once and then discarded.

### Determination of A1340 in Mouse Brain

Endogenous Aβ40 in mice was determined with the Meso Scale Discovery (MSD) 96-well MULTI-ARRAY human/rodent (4G8) Aβ40 Ultrasensitive Assay (#K110FTE-3, Meso Scale Discovery, Gaithersburg, USA). The assay was performed according to the manufacturer's instructions except for the calibration curve and the sample preparations. TritonX-100 (TX-100) soluble Aβ40 was extracted from forebrain with 1% TX-100 using a 50 μl aliquot of each 1:10 forebrain homogenate, mixed with 50 μl 2% TX-100 in TBS complete (20 mM Tris-HCl pH 7.4, 137 mM NaCl, 1× Complete [Protease Inhibitor Cocktail Tablets: 1 836 145, Roche Diagnostics GmbH, Penzberg Germany]) to reach a final concentration of 1% TX-100 and a 1:20 forebrain dilution. The samples were incubated for 15 min on ice and vortexed every 5 min. The samples were ultra-centrifuged (100000×g, 4° C., 15 min) and 50 μl of the clear supernatants were transferred to fresh tubes. For the Aβ40 assay the supernatants were further diluted 1:5 in 3% Blocker A solution (from kit) to a final forebrain dilution of 1:100 and applied to the plate.

The calibration curve was prepared in a corresponding dilution of 1% Blocker A solution spiked with synthetic Aβ1-40 peptide (1.56-100 pg/ml) except for non-transgenic mouse brain samples: In this case, the calibration curve was prepared in a correspondingly diluted APP knockout mouse forebrain spiked with synthetic Aβ1-40 peptide (1.56-100 pg/ml). For all samples and standards 25 μl were applied per well. For each determination duplicate wells were performed. The mean values from the duplicate wells were used for calculations. Since MSD did not provide quantification software, the relative units for samples and standards were imported into SOFTmax PRO 4.0 for calculation of standard curves and quantification of samples.

### Results

Effects on Body Weight and Fur Colour in C57BL/6 Mice Chronically Treated with NB-360 or Compound 1

Wild-type, naive mice (C57BL/6) were chronically treated for 8 weeks with Compound 1 or NB360 and body weight was measured every 3^{rd} day (Monday, Wednesday, Friday). No overall significant body weight difference of the treatment group compared to vehicle could be observed as well as no significant difference at the end of the study at day 56. Nevertheless, for the treatment group a significant body weight gain (body weight comparison of day 0 to day 56) could be observed.

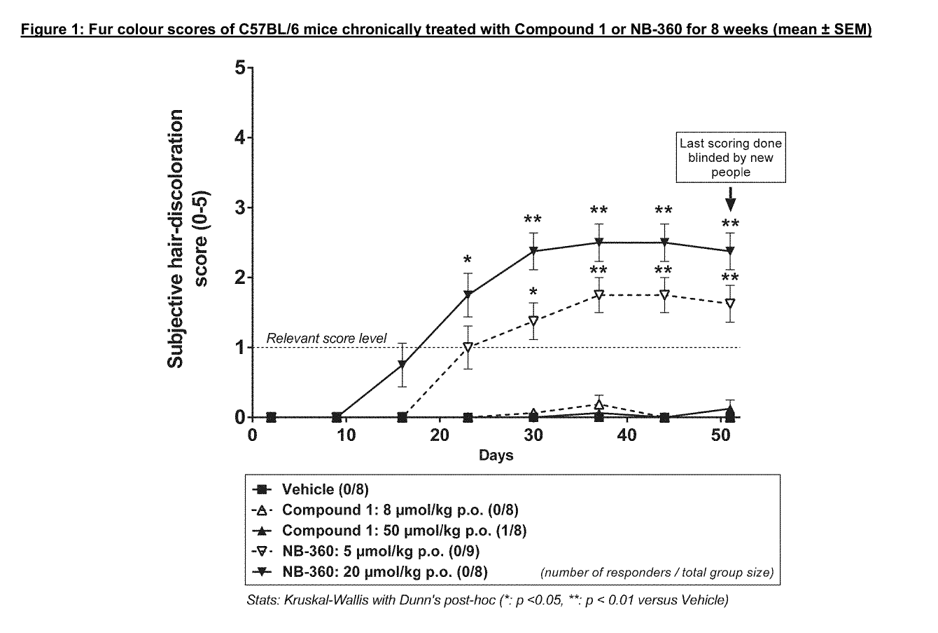

During the course of the study fur colour changes were observed in mice treated with NB-360. The black fur of C57BL/6 turned slowly grey in patches. These grey patches were visible on the ventral part of the animals while the dorsal part was unaffected. The appearance of grey patches was apparent after 3 weeks of treatment and present at different degrees in the high and low dose NB-360 group. A subjective scoring system was implemented to quantitate the fur discoloration. All animals in the NB-360 group showed fur discoloration. While the low dose NB-360 group (20 μmol/kg) showed only a slight but significant fur score change, the high dose NB-360 group (100 μmol/kg) displayed a more severe and profound fur color change, FIG. 1. Noticeably, the spread and increase of the fur discoloration reached a plateau after 5 weeks of NB-360 treatment without any furthermore changes. Importantly, there was no apparent fur color change detectable in the Compound 1 treatment groups.

### Exposure in Blood and Tissues

Compound 1 exposure in blood was determined at day 1 after the first dose, at interim at day 14, and at the end of the study after the last dose. Exposure at the last day was consistently lower than at the beginning of the experiment. Exposure was reduced by around 35% for Compound 1.

Exposure of Compound 1 over the last 24 hours, expressed as AUC₀₋₂₄ₕ in the different tissues, is summarized in Table 4. For blood, AUC was calculated form the data at 1, 4, 7, and 24 hours, as well as a ‘mini’ AUC only from the data at 4 and 24 hours. Comparison of the two values does not show a big difference. For tissue exposure, only data at 4 and 24 h were available. It was concluded that the ‘mini’ AUC sufficiently well represented the tissue exposure.

For both Compound 1 and NB-360, exposure in brain and skin was much higher than in blood, Table 4. In particular, the skin exposure was several fold higher than blood exposure. In addition, there seemed to be a higher exposure in ventral than in dorsal parts of the skin, especially for NB-360, Table 5. In all tissues, there was a good dose proportionality of the exposure.

TABLE 4
Compound AUC₀₋₂₄ₕ in various blood and brain tissues after last dose (day 56)

| | Dose | AUC_{blood} | AUC_{blood} | AUC_{brain} | AUC_{dorsal skin} | AUC_{ventral skin} |
|---|---|---|---|---|---|---|
| Comp. | (μmol/kg) | (μM · h)^{a} | (μM · h)^{b} | (μmol/kg · h)^{b} | (μmol/kg · h)^{b} | (μmol/kg · h)^{b} |
| NB-360 | 20 | 6.93 | 5.60 | 16.39 | 46.9^{c} | 104.81 |
| | 100 | 35.89 | 25.26 | 114.45 | 294 | 712.16 |
| Cmpd. 1 | 8 | 3.43 | 3.49 | 11.61 | 54.3 | 78.29 |
| | 50 | 28.93 | 18.53 | 88.93 | 271.6 | 308.7 |

^{a}AUC from 1, 4, 7, and 24 h time points;
^{b}AUC from 4 and 24 h time points;
^{c}n = 2 for 24 h

TABLE 5
Tissue exposures normalized to blood exposure of compounds (mean
of low and high dose)

| | rel. AUC | rel. | | Ratio ventral/ |
|---|---|---|---|---|
| Comp. | brain | AUC_{dorsal skin} | rel. AUC_{ventral skin} | dorsal |
| NB-360 | 2.8 | 7.5 | 17.4 | 2.3 |
| Cmpd. 1 | 3.2 | 12.6 | 16.8 | 1.3 |

Rel.: relative - normalized to AUC blood

### Amyloid-Beta Lowering in Mouse Brain

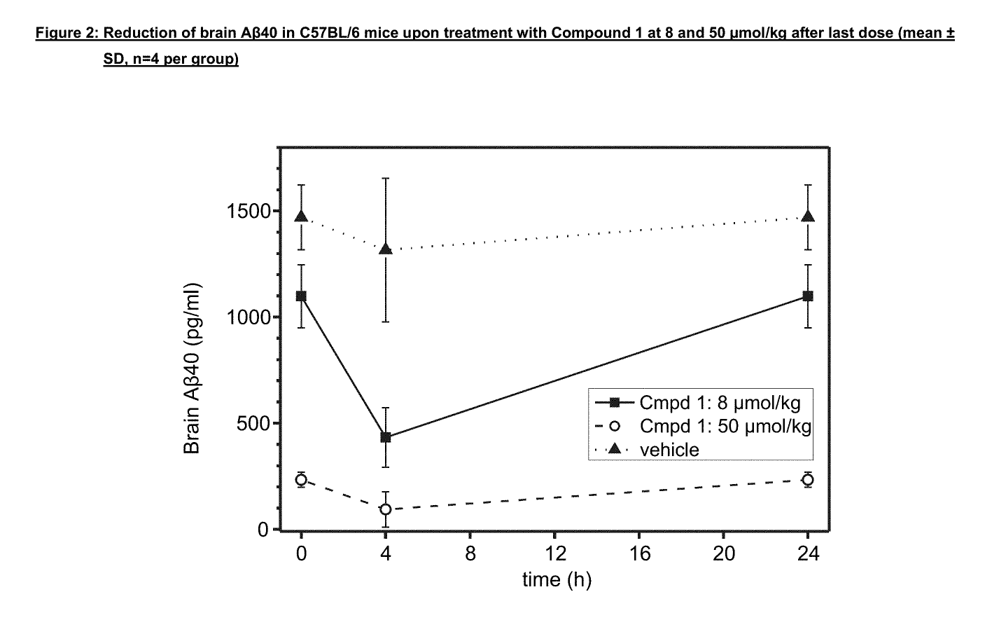

At the last day of the study, groups of n=4 mice were sacrificed 4 hours and 24 hours after having received the last dose. Forebrain was separated, and analyzed for β-amyloid peptide 1-40. Concentrations of Aβ40 for the vehicle and treatment groups are summarized in Table 6 and visualised in FIG. 2. The percent of reduction versus the corresponding vehicle treated group was calculated. Treatment resulted in a significant Aβ40 reduction 4 hours after the last dose. Compound 1 showed still 25% reduction of Aβ40 24 hours after the last dose relative to vehicle, but this was not significant. In the high dose group, Compound 1 showed significantly lower levels of Aβ40 24 hours after the last dose. The 50 μmoles/kg Compound 1 dose group showed an almost flat profile, with 80-90% Aβ40 reduction over the whole 24 hour time course.

TABLE 6
Effect of BACE inhibitor treatment on Aβ40 levels in
mouse brain (mean ± SD, n = 4) after last dose

| | | | | % | significance | Mini |
|---|---|---|---|---|---|---|
| | dose | | Average Aβ40 | reduction | vs vehicle | AUEC |
| | (μmol/kg) | time | brain (pg/g) | vs vehicle | same time | (%) |
| vehicle | n.a. | 4 | 1316.1 ± 338.0 | 100 | n.a. | |
| vehicle | n.a. | 24 | 1469.6 ± 152.8 | 100 | n.a. | |
| Compound 1 | 8 | 4 | 432.8 ± 140.5 | 67.1 | p < 0.01 | 46.2 |
| Compound 1 | 8 | 24 | 1098.6 ± 148.7 | 25.3 | n.s. | |
| Compound 1 | 50 | 4 | 93.4 ± 83.7 | 92.9 | p < 0.01 | 88.5 |
| Compound 1 | 50 | 24 | 233.8 ± 36.5 | 84.1 | p < 0.01 | |

n.s.: not significant

NB-360 is a dual BACE-1/BACE-2 inhibitor, as indicated by the BACE-1 and BACE-2 enzyme inhibition in vitro assays (Neumann U et al., (2015)) which give a 1.0-fold selectively for BACE-1 over BACE-2. In the same assays, Compound 1 was found to have a 3 fold selectivity for BACE-1 over BACE-2. In conclusion, moderate variations in enzyme selectivity and tissue distribution between Compound 1 and NB-360 are believed to have an effect on the occurrence of hair discoloration in chronic mouse studies. Despite being active in vivo, Compound 1 did not show signs of hair discoloration in mice.

### Example 3: Acute PK/PD Dose-Response Study of Compound 1 in APOE4-TR Mice

To investigate the effects of Compound 1 on APP metabolism in the human APOE4 context, PK/PD studies in transgenic mice carrying the human APOE4 allele were performed (mouse Apoe gene was replaced by human APOE4; APOE4-TR; (Knouff C et al., 1999)).

In this study, male and female APOE4-TR animals at the age of 3-5 months were treated acutely with Compound 1 at different doses (3, 10, 30 u mol/kg) and sacrificed at 4 h and 24 after treatment.

### Animals

Male and female transgenic homozygous APOE4-TR (B6.129P2-Apoe^{tm3(APOE*4)Mae} N8, Taconic, Model 001549, 3-5 months old, n=48) were obtained from Taconic.

### Dose Selection

Compound 1 was administered at 3, 10 and 30 μmol/kg.

### Compound Form, Formulation and Dosing

Compound 1 was formulated as a suspension. Vehicle or compound was given by oral administration in a volume of 10 ml/kg once. Vehicle: 0.1% Tween80 in 0.5% Methylcellulose in water.

TABLE 7
Treatment Groups

| | | | Day 1 | |
|---|---|---|---|---|
| Treatment | Dose at 0 h | n = | +4 h | +24 h |
| A | Compound 1: 3 μmol/kg | 12 | Sacrifice (n = 3 ♀ n = 3 | Sacrifice (n = 3 ♀ n = 3 |
| | | | ♂) | ♂) |
| B | Compound 1: 10 μmol/kg | 12 | Sacrifice (n = 3 ♀ n = 3 | Sacrifice (n = 3 ♀ n = 3 |
| | | | ♂) | ♂) |
| C | Compound 1: 30 μmol/kg | 12 | Sacrifice (n = 3 ♀ n = 3 | Sacrifice (n = 3 ♀ n = 3 |
| | | | ♂) | ♂) |
| V | Vehicle (0.1% Tween 80, | 12 | Sacrifice (n = 3 ♀ n = 3 | Sacrifice (n = 3 ♀ n = 3 |
| | 0.5% MC) | | ♂) | ♂) |

### Body Weight

Body weight was taken once before dosing.

### Ex Vivo Samples and Sample Harvest Methodology

Blood samples were used to analyze whole blood compound levels and were obtained from trunk blood at the day of necropsy into EDTA Eppendorf tubes (Milian S A, CatNoTOM-14, Fisher Scientific, Wohlen, Switzerland), or into serum tubes (CB300Z, Sarstedt, Nümbrecht, Germany).

Plasma for amyloid-β (Aβ) analysis was collected by centrifugation of EDTA blood (8000 rpm/6800×g, 15 min, 4° C.) and collected into protein Lo-Bind Eppendorf tubes (003 0108.116, Eppendorf, Hamburg, Germany).

All blood/plasma/serum samples were frozen on dry ice and stored at −80° C. until analysis.

Brain was removed immediately after decapitation, rinsed with saline and sectioned sagitally down the midline. The left cerebellum was used to analyze compound level and was placed into a glass tube (Chromacol, 125×5-SV T051, Welwyn Garden City, United Kingdom), weighed and frozen in dry-ice, the left half of the forebrain (without olfactory bulb) was used for Aβ analysis, and was frozen on a metal plate on dry ice and placed into protein Lo-bind tube (003 0108.116, Eppendorf, Hamburg, Germany). The right brain was fixed in 4% paraformaldehyde, washed in PBS and then embedded in paraffin for possible future histological analyses.

Tails were collected at the end of the study and stored at −20° C.

TABLE 8
Analysis of compound levels

| | |
|---|---|
| Analytical method | HPLC/MS/MS |
| Brain homogenization | Brain samples were mixed with 2 volumes of methanol/water (2/8 v/v) |
| method | and homogenized using the Precellys ® device. |
| Sample preparation | 30 μL of blood or brain homogenate were spiked with a generic internal |
| method | standard (labetalol) and subsequently mixed with 200 μL acetonitrile |
| | (protein precipitation). A 1 μL aliquot of the supernatant was injected into |
| | the LC/MS/MS system for analysis. |
| MS | AB Sciex Triple Quad 5500 (AB Sciex, Brugg AG, Switzerland); Turbo Ion |
| | Electrospray Ionisation in positive ion mode. |
| MS/MS methods | 1. [Compound 1]: |
| | 508.2 Da [M + H]⁺ → 139.1; 180.1; 375.1 Da/CE 85; 83; 43 eV |
| | 2. [Labetalol]: (Internal Standard) |
| | 329.0 [M + H]⁺ → 294.0 and 311.2 Da/CE 26 and 19 eV |
| HPLC | Waters Acquity UPBINARY (Waters/Dättwil AG, Switzerland) |
| HPLC columns | Synergi Kinetex C18, 50 * 2.1 mm, 2.6 μm (Phenomenex, Torrance, CA, |
| | U.S.A.) |
| Buffer A // Buffer C | A: H₂O + 0.1% formic acid // B: methanol/acetonitrile (1/1) + 0.1% |
| | formic acid |
| Gradients | Pump 1: 0.0′ 98% A, 2% B // 2.5′ 10% A, 90% B // 2.6′ 1% A, 99% B // 3′ |
| | 98% A, 2% B // 4′ 98% A, 2% B. |
| | Flow 0.0′-2.5′ = 400 μl/min // 2.6′-4.0′ = 500 μl/min |
| LLOQ | Compound 1: 0.4 ng/mL |
| Dynamic range: | 0.4 to 12500 ng/mL (Compound 1) |
| Calibration standards | Bias within the range −0.3 to −2.4% at the LLOQ and −12.3 to 14.4% at |
| | the other concentration levels |
| Quality Control | Bias within the range of −4.3 to 14.4% of all blood quality control |
| | samples. Bias within the range of 16.8 to 31.8% of all brain quality |
| | control samples: One brain QC was out of ±30% |
| Acceptance criteria in | Calibration standards: Bias within the range ±30% at the LLOQ and at |
| each run | ±20% at the other concentration levels. At least ¾ of the individual back- |
| | calculated values with at least one value at both extremes of the standard |
| | curve fulfilling the acceptance criteria. |
| | Quality Control samples: Bias within the range ±30% for at least ⅔ of |
| | the individual values. At least one value at each QC level fulfilling the |
| | acceptance criteria. |

### Analysis of Aβ40 in Mouse Brain and Aβ40 and Aβ42 in CSF

### Brain Homogenization

Frozen mouse forebrains were weighed and homogenized in 9 volumes (w/v) of ice-cold TBS-Complete (20 mM Tris-HCl pH 7.4, 137 mM NaCl, 1× Complete [Protease Inhibitor Cocktail Tablets: 1 836 145, Roche Diagnostics GmbH, Penzberg, Germany]) by sonication (90% duty cycle, output control 5, 40-55 pulses, [Sonifier 450, Branson]). After homogenization several 50 μl aliquots were prepared for analysis and were stored at −80° C.

### Preparation of Synthetic Aβ1-40 Solutions as Standards

Human Aβ1-40 trifluoroacetate salt (H 1194.1000, Bachem, Bubendorf, Switzerland) was used as calibration curve for Aβ1-40. It was solubilized in water-free DMSO (41647, Fluka) at a concentration of 1 mg/ml for approximately 30 min at room temperature (RT) and then visually checked for complete solubilization.

20×5 μl aliquots and 100 μl aliquots of the remaining solution were prepared in LoBind tubes (0030 108.094, Eppendorf, Hamburg, Germany), overlaid with nitrogen gas in order to protect the Aβ peptide from oxidation and stored at −80° C. For the calibration curves a 5 μl aliquot was used just once and then discarded.

### Determination of Aβ40 in Mouse Brain

Endogenous Aβ40 in mice was determined with the Meso Scale Discovery (MSD) 96-well MULTI-ARRAY human/rodent (4G8) Aβ40 Ultrasensitive Assay (#K110FTE-3, Meso Scale Discovery, Gaithersburg, USA). The assay was performed according to the manufacturer's instructions except for the calibration curve and the sample preparations. TritonX-100 (TX-100) soluble Aβ40 was extracted from forebrain with 1% TX-100 using a 50 μl aliquot of each 1:10 forebrain homogenate, mixed with 50 μl 2% TX-100 in TBS complete (20 mM Tris-HCl pH 7.4, 137 mM NaCl, 1× Complete [Protease Inhibitor Cocktail Tablets: 1 836 145, Roche Diagnostics GmbH, Penzberg Germany]) to reach a final concentration of 1% TX-100 and a 1:20 forebrain dilution. The samples were incubated for 15 min on ice and vortexed every 5 min. The samples were ultra-centrifuged (100000×g, 4° C., 15 min) and 50 μl of the clear supernatants were transferred to fresh tubes. For the Aβ40 assay the supernatants were further diluted 1:5 in 3% Blocker A solution (from kit) to a final forebrain dilution of 1:100 and applied to the plate.

The calibration curve was prepared in a corresponding dilution of 1% Blocker A solution spiked with synthetic Aβ1-40 peptide (1.56-100 pg/ml) except for non-transgenic mouse brain samples: In this case, the calibration curve was prepared in a correspondingly diluted APP knockout mouse forebrain spiked with synthetic Aβ1-40 peptide (1.56-100 pg/ml). For all samples and standards 25 μl were applied per well. For each determination duplicate wells were done. The mean values from the duplicate wells were used for calculations. Since MSD did not provide quantification software, the relative units for samples and standards were imported into SOFTmax PRO 4.0 for calculation of standard curves and quantification of samples.

### Results

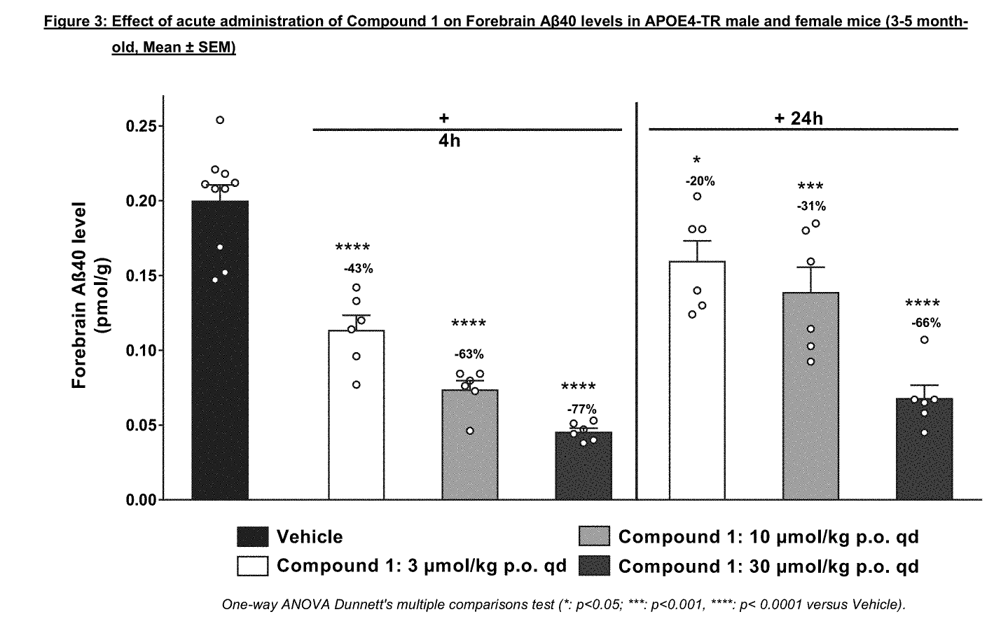

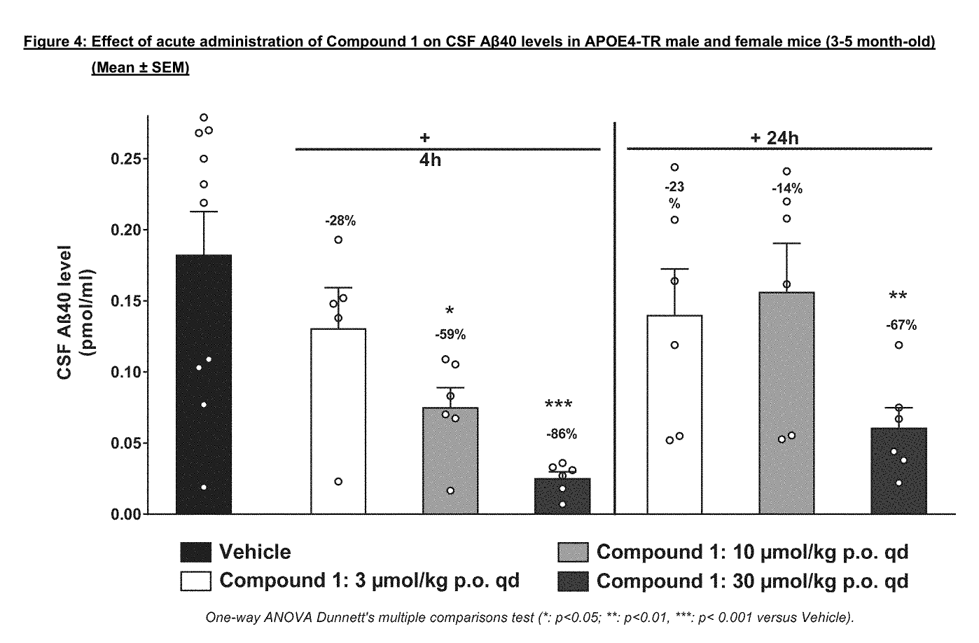

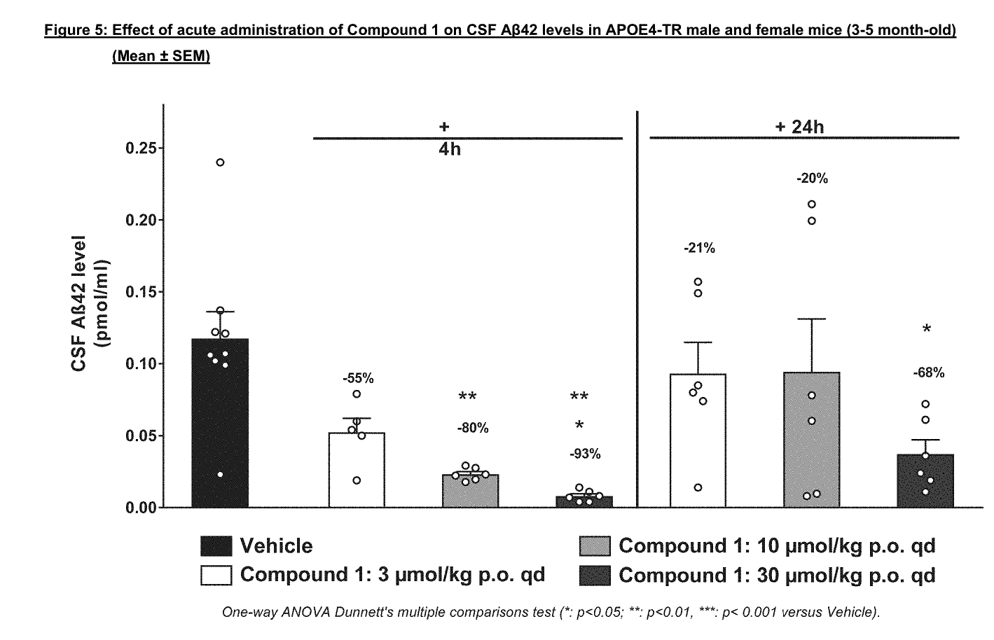

APOE4-TR mice (mouse Apoe gene was replaced by human APOE4) were acutely treated with three different doses (3, 10 and 30 μmol/kg) of the BACE inhibitor Compound 1. Animals were sacrificed 4 h and 24 h after last the last dose and forebrains were separated. Concentrations of Aβ40 and Aβ42 for the various groups are summarized in FIGS. 3, 4 and 5; and Tables 9, 10 and 11. The percent of reduction versus the vehicle treated group was calculated. All treatments resulted in a significant and dose-dependent Aβ40 reduction at 4 h and 24 h after the last dose, the effect ranged from 43-77% at 4 h and 20-66% at 24 h. For the two lower dose groups (3 and 10 μmol/kg) the Aβ40 lowering effect was significantly reduced at 4 h and 24, but substantially approximated baseline levels 24 h after the last dose. The high dose of Compound 1 (30 μmol/kg) showed an almost flat profile with 77-66% Aβ40 reduction over the whole 24 h time course.

TABLE 9
Effect of Compound 1 treatment on Aβ40 levels in APOE4-TR
mouse brain (n = 6 (n = 3 males, n = 3 females))

| | | | Average Aβ40 | | | |
|---|---|---|---|---|---|---|
| | Dosing | | brain | % reduction | Significance | Mini |
| Dose | Regime | Time | (pmol/g) ± SD | vs vehicle | vs vehicle | AUEC (%) |
| Vehicle | Acute | 4/24 | 0.200 ± 0.034 | n.a. | n.a. | n.a. |
| 3 μmol/kg | Acute | 4 | 0.114 ± 0.024 | 43 | p < 0.0001 | 32 |
| 3 μmol/kg | Acute | 24 | 0.160 ± 0.033 | 20 | p < 0.05 | |
| 10 μmol/kg | Acute | 4 | 0.074 ± 0.014 | 63 | p < 0.0001 | 47 |
| 10 μmol/kg | Acute | 24 | 0.139 ± 0.041 | 31 | p < 0.001 | |
| 30 μmol/kg | Acute | 4 | 0.046 ± 0.006 | 77 | p < 0.0001 | 72 |
| 30 μmol/kg | Acute | 24 | 0.068 ± 0.021 | 66 | p < 0.0001 | |

n.a.: not applicable,
vehicle: all vehicles combined

TABLE 10
Effect of Compound 1 treatment on Aβ40 levels in APOE4-TR
mouse CSF (n = 6 (n = 3 males, n = 3 females))

| | Dosing | | Average Aβ40 CSF | % reduction | Significance | Mini |
|---|---|---|---|---|---|---|
| Dose | Regime | Time | (pmol/ml) ± SD | vs vehicle | vs vehicle | AUEC (%) |
| Vehicle | Acute | 4/24 | 0.1826 ± 0.0956 | n.a. | n.a. | n.a. |
| 3 μmol/kg | Acute | 4 | 0.1308 ± 0.06381 | 28 | ns | 26 |
| 3 μmol/kg | Acute | 24 | 0.1402 ± 0.07911 | 23 | ns | |
| 10 μmol/kg | Acute | 4 | 0.07527 ± 0.03356 | 59 | p < 0.05 | 37 |
| 10 μmol/kg | Acute | 24 | 0.1565 ± 0.08348 | 14 | ns | |
| 30 μmol/kg | Acute | 4 | 0.02533 ± 0.01093 | 86 | p < 0.001 | 77 |
| 30 μmol/kg | Acute | 24 | 0.06083 ± 0.03445 | 67 | p < 0.01 | |

n.a.: not applicable,
vehicle: all vehicles combined,
ns: not significant

TABLE 11
Effect of Compound 1 treatment on Aβ42 levels in APOE4-TR
mouse CSF (n = 6 (n = 3 males, n = 3 females))

| | Dosing | | Average Aβ42 CSF | % reduction | Significance | Mini |
|---|---|---|---|---|---|---|
| Dose | Regime | Time | (pmol/ml) ± SD | vs vehicle | vs vehicle | AUEC (%) |
| Vehicle | Acute | 4/24 | 0.1174 ± 0.05610 | n.a. | n.a. | n.a. |
| 3 μmol/kg | Acute | 4 | 0.0524 ± 0.02173 | 55 | ns | 38 |
| 3 μmol/kg | Acute | 24 | 0.09317 ± 0.05304 | 21 | ns | |
| 10 μmol/kg | Acute | 4 | 0.02318 ± 0.00446 | 80 | p < 0.01 | 50 |
| 10 μmol/kg | Acute | 24 | 0.09438 ± 0.09019 | 20 | ns | |
| 30 μmol/kg | Acute | 4 | 0.0080 ± 0.00395 | 93 | p < 0.001 | 81 |
| 30 μmol/kg | Acute | 24 | 0.03717 ± 0.02438 | 68 | p < 0.05 | |

n.a.: not applicable,
vehicle: all vehicles combined,
ns: not significant

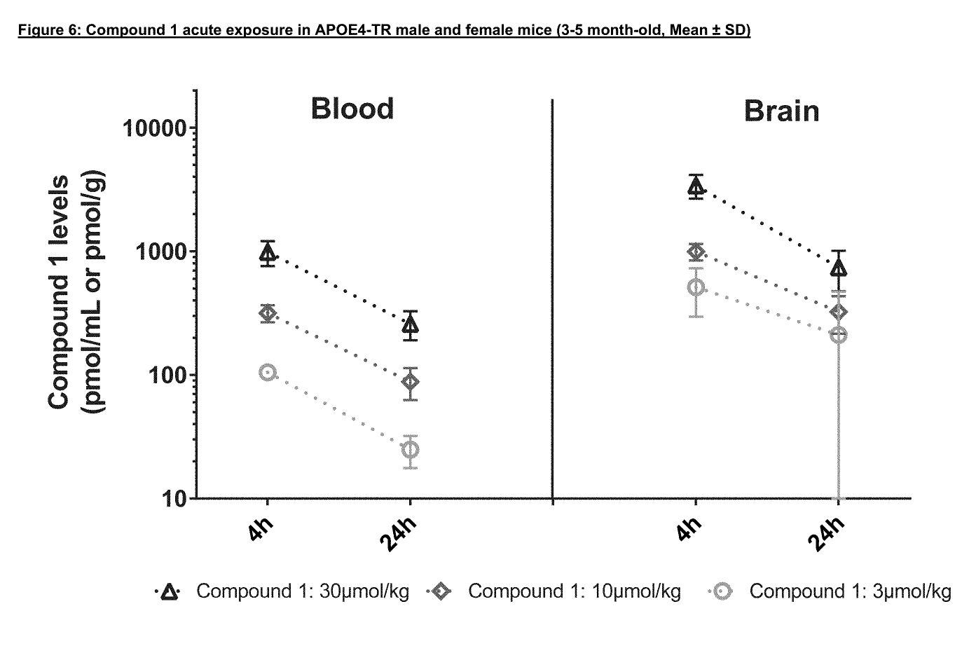

PK data are shown in FIG. 6 and Table 12 at 4 h and 24 h for acute dosing in blood and brain. Exposure of Compound 1 over 24 h, expressed as AUC₀₋₂₄ₕ in blood and brain is summarized in Table 13. Compound 1 exposure in blood and brain was dose proportional and displayed the expected minor decline in compound level after 24 h, which again, was dose proportional. The compound exposure in brain was much higher than in blood. The brain blood ratio was similar for 3, 10 and 30 μmol/kg dose groups with 5, 3 and 4 at 4 h and 9, 4, and 3 at 24 h, respectively. The exposure ratio at 4 h/24 h was calculated which allows the comparison of the decline of compound exposure at the different doses (Table 12). Compound 1 had a moderate 2-5 fold exposure reduction, without big differences between the different doses and between blood and brain.

TABLE 12
Compound 1 levels in blood and brain of APOE4-TR
mice (n = 6 (n = 3 males, n = 3 females)

| | | | Mean Blood | Exposure | Mean Brain | Exposure |
|---|---|---|---|---|---|---|
| | | | (pmol/ml) ± | ratios 4 h/24 h | (pmol/g) ± | ratios 4 h/24 h |
| Treatment | Dosing | Time | SD | (Blood) | SD | (Brain) |
| 3 μmol/kg | Acute | 4 | 105 ± 8 | 4.2 | 512 ± 216 | 2.4 |
| 3 μmol/kg | Acute | 24 | 25 ± 7 | | 210 ± 262 | |
| 10 μmol/kg | Acute | 4 | 317 ± 50 | 3.6 | 995 ± 151 | 3.1 |
| 10 μmol/kg | Acute | 24 | 88 ± 25 | | 324 ± 109 | |
| 30 μmol/kg | Acute | 4 | 985 ± 224 | 3.8 | 3409 ± 741 | 4.6 |
| 30 μmol/kg | Acute | 24 | 259 ± 68 | | 743 ± 267 | |

TABLE 13
Compound 1 AUC_{0-24 h} in blood and brain of APOE4-TR mice

| | | AUC_{blood} | AUC_{brain} |
|---|---|---|---|
| Treatment | Dosing | (μM · h) | (μmol/kg · h) |
| 3 μmol/kg | Acute | 1.56 | 8.67 |
| 10 μmol/kg | Acute | 4.86 | 15.83 |
| 30 μmol/kg | Acute | 14.92 | 49.82 |

AUC from 4 h and 24 h time points

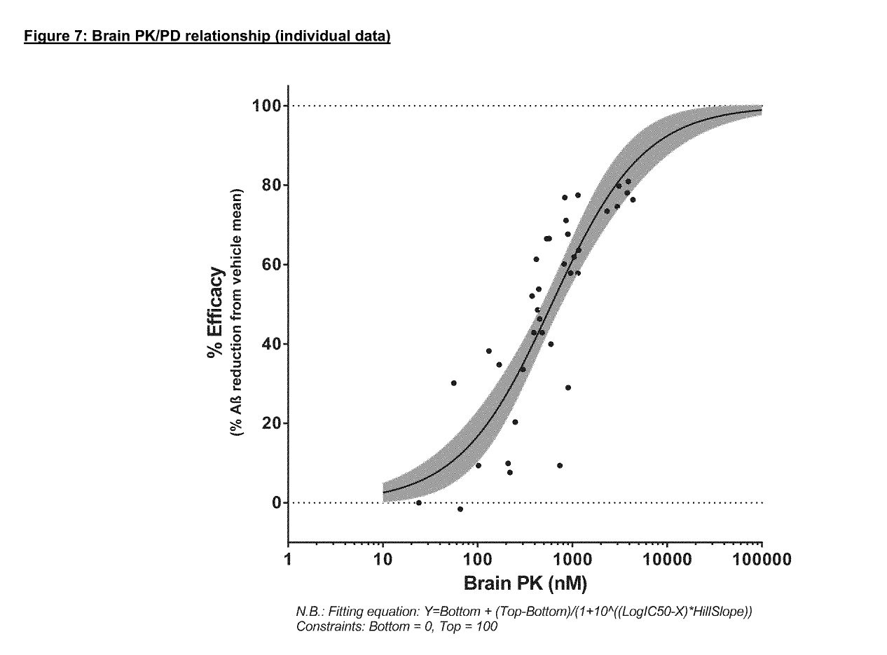

The brain pharmacokinetic/pharmacodynamic relationship for the individual animals for all dose groups is shown in FIG. 7. There was a clear PK/PD relationship apparent for Compound 1; at low compound levels the Aβ reduction efficacy was minimal whereas at high compound levels a maximum efficacy effect was detected.

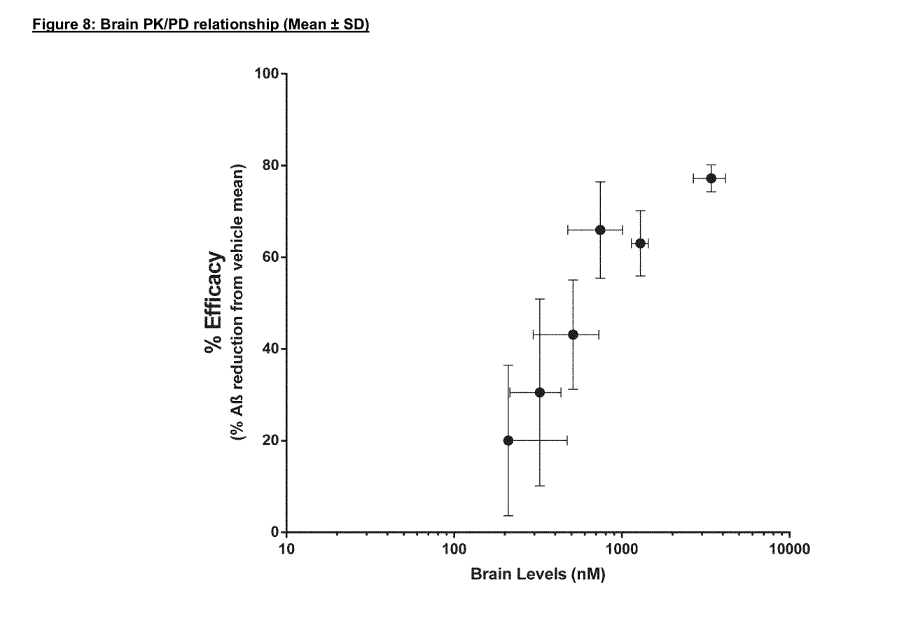

FIG. 8 displays the PK/PD relationship for the averaged values at the different doses. Again, the exposure dependent effect on Aβ reduction is apparent, with a clear minimal and maximal efficacy effect.

### Conclusion

Studies presented in this experimental example demonstrate that Compound 1 is an orally-available, centrally active and potent inhibitor of BACE in vivo in APOE4-TR mice. APOE4-TR mice that express human APOE4 from the mouse endogenous Apoe locus were used to investigate PK/PD relationship of Compound 1. ApoE4 has been implicated to be a high risk factor for Alzheimer's disease and APOE4-TR mice resemble the ApoE4 effect in the Alzheimer's brain.

The PK properties of Compound 1 in APOE4-TR mice did not differ to those observed in wildtype mice. A dose-dependent Compound 1 exposure in blood and brain, with much higher brain levels, was observed. Furthermore, the exposure decrease after 24 h was similar to that observed in wildtype mice. Compound 1 at 30 μmol/kg resulted in the maximal effect on Aβ reduction (>70%) in the brain of APOE4-TR, with similar extent lasting over 24 h for acute dosing. The PK/PD relationship was very comparable to wildtype mice and rats. There was a slightly lower maximal efficacy effect on Aβ reduction in the brain apparent in APOE4-TR mice at the highest dose (30 μmol/kg). This might be due to a lower clearance rate of amyloid-β observed in APOE-4 TR mice (Castellano J M et al., 2011).

### Example 4: First-in-Human Study

This study has been clinically completed and was a randomised, double-blind, placebo-controlled, single and multiple ascending oral dose study to primarily assess the safety and tolerability as well as the pharmacokinetics and pharmacodynamics of Compound 1 in healthy adult and elderly subjects. The purpose of this study was to determine the single and multiple maximum tolerated dose of Compound 1 and to assess the pharmacokinetic/pharmacodynamic (PK/PD) relationship using Aβ in CSF as primary PD biomarker.

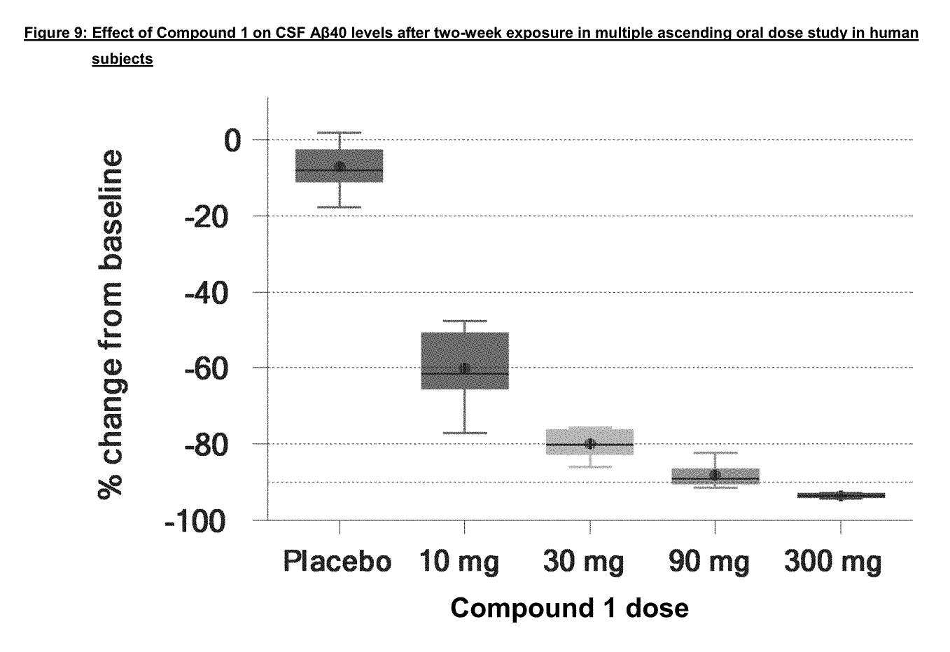

In healthy elderly subjects 60 years of age, the highest tested doses of 750 mg single dose and 300 mg QD over two weeks were determined to be safe and tolerated. Pharmacodynamic assessments using Aβ concentrations in CSF as primary biomarker of drug action were also been applied in healthy elderly subjects. Dose-dependent lowering of Aβ40 concentrations was determined up to approximately 80% and 90% after single and multiple dosing, respectively (Tables 14 and 15, FIG. 9).

TABLE 14
Aβ40 in CSF - Summary of percent change from baseline across time

| | | Single-dose | | | | |
|---|---|---|---|---|---|---|
| Time | | | Cmpd 1 | Cmpd 1 | Cmpd 1 | Cmpd 1 |
| (hours) | | Placebo | 10 mg | 90 mg | 300 mg | 750 mg |
| 12 h | N | 12 | 6 | 6 | 8 | 8 |
| | Mean (SD) | 1.6 (6.66) | −11.6 (8.31) | −14.2 (35.83) | −35.8 (16.40) | −36.2 (13.58) |
| | Median | 0.2 | −12.5 | −28.0 | −44.1 | −38.6 |
| | Min-Max | −7-16 | −21-1 | −36-58 | −54-−13 | −53-−10 |
| 24 h | N | 12 | 6 | 5 | 8 | 7 |
| | Mean (SD) | −2.2 (12.84) | −15.0 (10.21) | −44.8 (12.48) | −68.3 (10.65) | −67.4 (15.54) |
| | Median | −2.3 | −14.3 | −49.2 | −72.5 | −70.6 |
| | Min-Max | −22-22 | −31-−4 | −55-−25 | −79-−52 | −82-−36 |
| 34 h | N | 11 | 6 | 5 | 8 | 7 |
| | Mean (SD) | 5.1 (14.83) | −9.7 (14.72) | −50.0 (8.87) | −78.3 (4.75) | −79.1 (8.88) |
| | Median | 4.1 | −12.5 | −45.4 | −79.3 | −82.3 |
| | Min-Max | −22-29 | −25-10 | −60-−41 | −84-−69 | −87-−60 |

TABLE 15
Aβ40 in CSF Summary of percent change from baseline on Day 15 (24 h post last
dose)

| | | Multiple-dose | | | |
|---|---|---|---|---|---|
| | | Cmpd 1 | Cmpd 1 | Cmpd 1 | Cmpd 1 |
| | Placebo | 10 mg | 30 mg | 90 mg | 300 mg |
| N | 16 | 8 | 8 | 8 | 8 |
| Mean (SD) | −7.2 (6.04) | −60.1 (9.91) | −80.0 (3.89) | −88.2 (3.07) | −93.6 (0.61) |
| Median | −8.0 | −61.5 | −80.2 | −89.1 | −93.5 |
| Min; Max | −18; 2 | −77; −48 | −86; −76 | −92; −82 | −94; −93 |

### Example 5: 3-Month Dose-Ranging Safety and Tolerability Study

Compound 1 was administered to healthy elderly subjects 60 years or over in a Phase I clinical dose-ranging safety and tolerability study. This study is listed in ClinicalTrials.gov under the NCT02576639 Identifier code.

This randomized, double-blind, placebo-controlled study had a parallel-group design and Compound 1 was administered as once-daily, oral doses to five treatment groups (Compound 1: 2 mg, 10 mg, 35 mg or 85 mg QD and placebo).

The primary purpose of this study was to expand on previous safety and tolerability data obtained over 2-week and 4-week duration in the first in human study and thereby allow initiation of future long-term efficacy trials in subjects at risk of AD. In addition, data relevant for Pharmacokinetic/Pharmacodynamic modeling was obtained in order to support dose selection decisions for future efficacy studies.

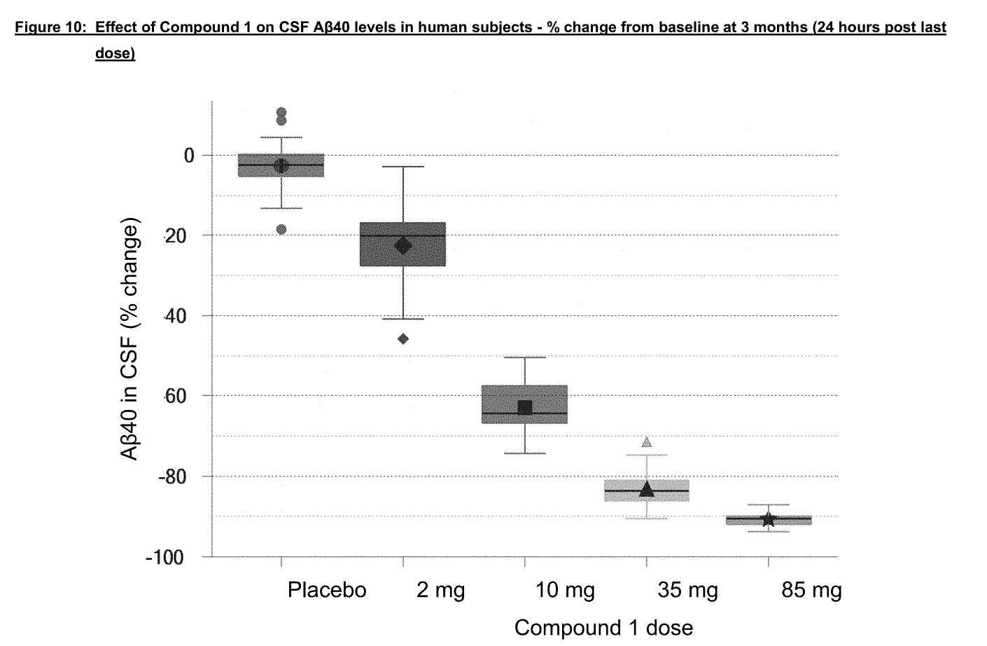

In this study, Compound 1 was found to be safe and tolerated at once-daily doses of 2, 10, 35 and 85 mg over three months. The pharmacodynamics effects of Compound 1 administration on CSF Aβ levels are shown in Table 16 and FIG. 10. The extent of Aβ lowering was stable over time with PD steady-state being reached after approximately 2-3 weeks.

TABLE 16
Aβ in CSF at month 3 - Analysis of percent change from baseline of Aβ38, Aβ40,
and Aβ42

| | | | | Difference (90% CI) | |
|---|---|---|---|---|---|
| Parameter | Treatment | N | LSmean (SE) | Cmpd 1 − Placebo | P-value |
| Aβ38 (pg/mL) | Placebo | 21 | −2.41 (1.430) | | |
| | Cmpd 1: 2 mg | 22 | −20.58 (1.397) | −18.17 (−21.49, −14.85) | <.001 |
| | Cmpd 1: 10 mg | 21 | −62.60 (1.430) | −60.18 (−63.54, −56.83) | <.001 |
| | Cmpd 1: 35 mg | 23 | −82.96 (1.366) | −80.55 (−83.83, −77.27) | <.001 |
| | Cmpd 1: 85 mg | 20 | −89.23 (1.470) | −86.81 (−90.22, −83.41) | <.001 |
| Aβ40 (pg/mL) | Placebo | 21 | −2.79 (1.389) | | |
| | Cmpd 1: 2 mg | 22 | −22.66 (1.355) | −19.87 (−23.09, −16.65) | <.001 |
| | Cmpd 1: 10 mg | 21 | −62.96 (1.388) | −60.18 (−63.43, −56.92) | <.001 |
| | Cmpd 1: 35 mg | 23 | −83.19 (1.325) | −80.41 (−83.59, −77.22) | <.001 |
| | Cmpd 1: 85 mg | 20 | −90.40 (1.429) | −87.62 (−90.93, −84.30) | <.001 |
| Aβ42 (pg/mL) | Placebo | 21 | −2.76 (1.310) | | |
| | Cmpd 1: 2 mg | 22 | −24.00 (1.277) | −21.24 (−24.28, −18.21) | <.001 |
| | Cmpd 1: 10 mg | 21 | −64.15 (1.308) | −61.39 (−64.47, −58.31) | <.001 |
| | Cmpd 1: 35 mg | 23 | −82.49 (1.250) | −79.73 (−82.73, −76.73) | <.001 |
| | Cmpd 1: 85 mg | 20 | −89.38 (1.348) | −86.62 (−89.76, −83.49) | <.001 |

Adjusted Lsmeans, difference in Lsmeans, 90% CI and P-value were obtained from an ANCOVA model with treatment as a fixed effect, and baseline AB level as covariat

The pharmacokinetic parameters of Compound 1 following three months (91 days) daily dosing at 2, 10, 35 and 85 mg are shown in Table 17.

TABLE 17
Compound 1 pharmacokinetic parameters on Day 91

| | Cmax, ss (ng/mL) | | |
|---|---|---|---|
| | 10 mg qd. | 35 mg qd. | 85 mg qd. |
| Results | 80 | 229 | 602 |
| Mean (CV %) | (36) | (33) | (25) |

The Cmax,ss value represents the maximum plasma steady state concentration of Compound 1 following 91 days of once daily (qd) dosing at the specified dose. “CV %” represents the percentage coefficient of variation.

Based on these results, a once daily dose of 15 mg of Compound 1 is expected to result in a plasma Cmax,ss value of between 70 and 170 ng/ml, and a once daily dose of 50 mg of Compound 1 is expected to result in a plasma Cmax,ss value of between 200 and 500 ng/ml.

Based on the data presented in Example 4 and 5, pharmacometric modelling predicts a daily dose of 50 mg to reach 80% CSF Aβ40 lowering and a dose of 15 mg to achieve 60% CSF Aβ40 lowering, in 90% of the subjects.

### Example 6: Effect of ApoE4 Genotype on Response to Treatment with Compound 1

In the completed first-in-human and 3-month dose-ranging safety and tolerability clinical studies described in Examples 5 and 6, Aβ concentrations in CSF were obtained by means of lumbar punctures before the first dose (baseline) and respectively after 2 weeks and 3 months of multiple dosing. ApoE4 genotype also was obtained in the subjects who consented. The percent change from baseline in Aβ40 and Aβ42 concentrations was calculated in subjects who took the study treatment and had no major protocol deviation with potential impact on the evaluation of the pharmacodynamic effect. Tables 18 to 21 below provide summary statistics of the percent change from baseline by treatment group and ApoE genotype (E4 heterozygotes versus E4 non-carriers). Only one subject with CSF data was E4 homozygote (from the 3-month dose-ranging safety and tolerability study). This subject was treated with placebo and showed 11% decrease in both Aβ40 and Aβ42 concentrations and is not included in the tables below. The data shows that there is no difference in CSF Aβ40 and Aβ42 response to treatment with Compound 1 between ApoE4 carriers and non-carriers.

TABLE 18
Aβ40 % change from baseline by ApoE genotype and Compound 1
treatment group at 3 months

| | | E4 heterozygotes | E4 non-carriers |
|---|---|---|---|
| Placebo | n | 9 | 8 |
| | Mean (SD) | −3 (7) | −2 (5) |
| | Median | −2 | −1 |
| | Range | −19; 9 | −13; 5 |
| Compound 1: 2 mg | n | 10 | 12 |
| | Mean (SD) | −21 (11) | −24 (9) |
| | Median | −18 | −22 |
| | Range | −46; −3 | −41; −10 |
| Compound 1: 10 mg | n | 3 | 15 |
| | Mean (SD) | −67 (7) | −64 (6) |
| | Median | −64 | −65 |
| | Range | −75; −61 | −73; −54 |
| Compound 1: 35 mg | n | 8 | 12 |
| | Mean (SD) | −84 (3) | −82 (5) |
| | Median | −85 | −83 |
| | Range | −88; −79 | −91; −72 |
| Compound 1: 85 mg | n | 2 | 17 |
| | Mean (SD) | −91 (0) | −91 (2) |
| | Median | −91 | −90 |
| | Range | −91; −91 | −94; −87 |

TABLE 19
Aβ42 % change from baseline by ApoE genotype and Compound 1
treatment group at 3 months

| | | E4 heterozygotes | E4 non-carriers |
|---|---|---|---|
| Placebo | n | 9 | 8 |
| | Mean (SD) | −2 (4) | −2 (7) |
| | Median | −0 | −1 |
| | Range | −10; 4 | −12; 8 |
| Compound 1: 2 mg | n | 10 | 12 |
| | Mean (SD) | −23 (9) | −24 (9) |
| | Median | −23 | −21 |
| | Range | −44; −8 | −42; −12 |
| Compound 1: 10 mg | n | 3 | 15 |
| | Mean (SD) | −66 (7) | −65 (5) |
| | Median | −63 | −66 |
| | Range | −74; −62 | −75; −56 |
| Compound 1: 35 mg | n | 8 | 12 |
| | Mean (SD) | −83 (5) | −81 (6) |
| | Median | −85 | −83 |
| | Range | −89; −72 | −88; −68 |
| Compound 1: 85 mg | n | 2 | 17 |
| | Mean (SD) | −88 (2) | −90 (2) |
| | Median | −88 | −90 |
| | Range | −90; −87 | −92; −84 |

TABLE 20
Aβ40 % change from baseline by ApoE genotype and Compound 1
treatment group at 2 weeks in first-in-human clinical study

| | | E4 heterozygotes | E4 non-carriers |
|---|---|---|---|
| Placebo | n | 5 | 9 |
| | Mean (SD) | −12 (3) | −6 (6) |
| | Median | −11 | −4 |
| | Range | −18; −9 | −17; 2 |
| Compound 1: 10 mg | n | 3 | 4 |
| | Mean (SD) | −56 (10) | −59 (7) |
| | Median | −53 | −61 |
| | Range | −67; −49 | −64; −48 |
| Compound 1: 30 mg | n | 0 | 7 |
| | Mean (SD) | | −80 (4) |
| | Median | | −81 |
| | Range | | −86; −76 |
| Compound 1: 90 mg | n | 2 | 5 |
| | Mean (SD) | −90 (2) | −89 (2) |
| | Median | −90 | −90 |
| | Range | −92; −89 | −91; −86 |
| Compound 1: 300 mg | n | 1 | 6 |
| | Mean (SD) | −93 | −94 (1) |
| | Median | −93 | −94 |
| | Range | −93; −93 | −94; −93 |

TABLE 21
Aβ42 % change from baseline by ApoE genotype and Compound 1
treatment group at 2 weeks in first-in-human clinical study

| | | E4 heterozygotes | E4 non-carriers |
|---|---|---|---|
| Placebo | n | 5 | 9 |
| | Mean (SD) | −12 (4) | −4 (10) |
| | Median | −9 | −4 |
| | Range | −17; −8 | −18; 17 |
| Compound 1: 10 mg | n | 3 | 4 |
| | Mean (SD) | −59 (11) | −58 (11) |
| | Median | −64 | −59 |
| | Range | −67; −47 | −70; −46 |
| Compound 1: 30 mg | n | 0 | 7 |
| | Mean (SD) | | −80 (6) |
| | Median | | −82 |
| | Range | | −86; −72 |
| Compound 1: 90 mg | n | 2 | 5 |
| | Mean (SD) | −87 (2) | −86 (5) |
| | Median | −87 | −87 |
| | Range | −89; −86 | −90; −78 |
| Compound 1: 300 mg | n | 1 | 6 |
| | Mean (SD) | −89 | −92 (1) |
| | Median | −89 | −91 |
| | Range | −89; −89 | −93; −90 |

### Example 7: Chronic Therapeutic Treatment of Plaque-Bearing Male APP23 Mice with the BACE Inhibitor Compound 1

### SUMMARY

Compound 1 was chronically administrated to APP23 transgenic mice at plaque bearing age (12 months) for 6 months, at two doses. Compared with a group that received vehicle only, the administration of Compound 1 at 0.03 g/kg food resulted in a slight, and the administration of 0.3 g/kg food resulted in a strong reduction of amyloid-β 40 and 42 compared to the vehicle group. The amount of Aβ in the mice brains was similar to the mice at baseline (12 months of age). Soluble Aβ in plasma and CSF were only significantly reduced in the high dose group. Plaque load as detected by immunohistochemistry, was also slightly (˜20%) reduced in the low dose group, and strongly (˜70%) reduced in the high dose group. The number of small, medium and large plaques responded equally to the treatment. The number of activated astrocytes was determined by GFAP staining. Total GFAP immunoreactivity was reduced by treatment with Compound 1 in a dose-dependent manner. While the majority of GFAP positive astrocytes was not associated with plaques, plaque associated astrocytes responded stronger to the Compound 1 treatment, compared to those distal from the plaques. Activated microglia cells were detected by staining with IBA1. The number of IBA1 positive microglia was dose dependently reduced by Compound 1 treatment. Microglia in close vicinity to amyloid plaques were more reduced by the treatment compared to microglia distal from plaques.

In summary, Compound 1 treatment showed a dose-dependent reduction of brain amyloid-β load, compared to untreated vehicle, and a correlating reduction of two neuroinflammation markers, the numbers of activated astrocytes and microglia cells in the mouse brains.

### Methods

### Animals and Dose Selection

Male transgenic, heterozygous APP23 (B6,D2-Tg(Thy1App)23Sdz (Sturchler-Pierrat C et al., 1997), 12-14 months old, n=64) were treated with 0.3 g/kg or at 0.03 g/kg Compound 1 in food pellets.

TABLE 22
Treatment Groups

| Group | Treatment | n = |
|---|---|---|
| A | Compound 1: 0.03 g/kg of food | 18 |
| B | Compound 1: 0.3 g/kg of food | 18 |
| C | Vehicle/Control | 18 |
| D | Baseline | 10 |

3 treatment groups, n = 18 mice per treatment group;
1 baseline group, n = 10

Ex Vivo Samples and Sample Harvest Methodology

Blood samples were used to analyze whole blood compound levels and were obtained from trunk blood at the day of necropsy into EDTA Eppendorf tubes (Milian S A, CatNoTOM-14, Fisher Scientific, Wohlen, Switzerland), or into serum tubes (CB300Z, Sarstedt, Nümbrecht, Germany).

Plasma for amyloid-β (A8) analysis was collected by centrifugation of EDTA blood (8000 rpm/6800×g, 15 min, 4° C.) and collected into protein Lo-Bind Eppendorf tubes (003 0108.116, Eppendorf, Hamburg, Germany).

All blood/plasma/serum samples were frozen on dry ice and stored at −80° C. until analysis.

Brain was removed immediately after decapitation, rinsed with saline and sectioned sagitally down the midline. The left half of the brain was used to analyze compound level and was placed into a glass tube (Chromacol, 125×5-SV T051, Welwyn Garden City, United Kingdom), weighed and frozen in dry-ice, the left half of the forebrain (without olfactory bulb) was used for Aβ analysis, and was frozen on a metal plate on dry ice and placed into protein Lo-bind tube (003 0108.116, Eppendorf, Hamburg, Germany).

Tails were collected at the end of the study and stored at −20° C.

### Analysis of Compound Levels

Compound 1 levels in biological samples were quantified in blood and brain by liquid chromatography/tandem mass spectrometry (HPLC/MS/MS). Brain samples were mixed with 2 volumes of KH₂PO₄ buffer and homogenized using the Covaris® device. Either 30 μL of blood or brain homogenate were spiked with a structurally related internal standard and subsequently mixed with an at least 6-fold excess volume acetonitrile for protein precipitation. The supernatant was injected directly into the LC/MS/MS system for analysis.

TABLE 23
Instrumental conditions for blood and brain samples

| | |
|---|---|
| Analytical method | HPLC/MS/MS |
| MS | Sciex QTrap 5500; |
| | Heated Electrospray Ionisation in positive ion mode. |
| MS/MS methods | 514.0 Da [M + H]⁺ 140.1/180.1 Da/CE 53/85 eV |
| HPLC | Flux Rheos Allegro (Thermo Scientific/Reinach BL, Switzerland) |
| HPLC columns | Phenomenex Kinetix C8 50 * 2.1 mm, 2.6 uM (Phenomenex, Torrance, |
| | CA, U.S.A.) |
| Buffer A // Buffer C | A: H₂O + 0.1% formic acid // C: acetonitrile + 0.1% formic acid |
| Gradients | Pump 1: 0.0′ 98% A, 2% C // 0.3′ 70% A, 30% C // 7.5′ 40% A, 60% C // |
| | 9.5′ 1% A, 99% C // 10.25′ 1% A, 99% C // 10.3′ 99% A, 1% C // 12.3′ |
| | 99% A, 1% C. Flow 300-350 μl/min |
| Acceptance criteria in | Calibration standards: Bias within the range ±20% at the LLOQ and at |
| each run | ±15% at the other concentration levels. At least ¾ of the individual |
| | back-calculated values with at least one value at both extremes of the |
| | standard curve fulfilling the acceptance criteria. |
| | Quality Control samples: Bias within the range ±30% for at least ⅔ of |
| | the individual values. At least one value at each QC level fulfilling the |
| | acceptance criteria. |
| | Dynamic range: 0.4 to 12500 ng/mL |

### Analysis of Aβ40 and Aβ42 in Mouse Tissue

### Brain Homogenization

Frozen mouse forebrains were weighed and homogenized in 9 volumes (w/v) of ice-cold TBS-Complete (20 mM Tris-HCl pH 7.4, 137 mM NaCl, 1× Complete [Protease Inhibitor Cocktail Tablets: 1 836 145, Roche Diagnostics GmbH, Penzberg, Germany]) by sonication (90% duty cycle, output control 5, 40-55 pulses, [Sonifier 450, Branson]). After homogenization several 50 μl aliquots were prepared for analysis and were stored at −80° C.

### Preparation of Synthetic Aβ Solutions as Standards

Human Aβ peptide (1-40) trifluoroacetate salt (H 1194.1000, Bachem, Bubendorf, Switzerland) was used as calibration curve for Aβ1-40. It was solubilized in water-free DMSO (41647, Fluka) at a concentration of 1 mg/ml for approximately 30 min at room temperature (RT) and then visually checked for complete solubilization.

20×5 μl aliquots and 100 μl aliquots of the remaining solution were prepared in LoBind tubes (0030 108.094, Eppendorf, Hamburg, Germany), overlaid with nitrogen gas in order to protect the Aβ peptide from oxidation and stored at −80° C. For the calibration curves a 5 μl aliquot was used just once and then discarded.

### Determination of Triton X-100 Soluble Aβ in APP23 Mouse Brain

Human Aβ40 and 42 in mice was determined with the Meso Scale Discovery (MSD) 96-well MULTI-ARRAY human/rodent (6E10) Aβ40/42 Assay (Meso Scale Discovery, Rockville, Md., USA). The assay was performed according to the manufacturer's instructions except for the calibration curve and the sample preparations. TritonX-100 (TX-100) soluble Aβ40 and 42 was extracted from forebrain with 1% TX-100 using a 50 μl aliquot of each 1:10 forebrain homogenate, mixed with 50 μl 2% TX-100 in TBS complete (20 mM Tris-HCl pH 7.4, 137 mM NaCl, 1× Complete [Protease Inhibitor Cocktail Tablets: 1 836 145, Roche Diagnostics GmbH, Penzberg Germany]) to reach a final concentration of 1% TX-100 and a 1:20 forebrain dilution. The samples were incubated for 15 min on ice and vortexed every 5 min. The samples were ultra-centrifuged (100000×g, 4° C., 15 min) and 50 μl of the clear supernatants were transferred to fresh tubes. The supernatants were further diluted 1:5 in 3% Blocker A solution (from kit) to a final forebrain dilution of 1:100 and applied to the plate.

The calibration curve was prepared in a corresponding dilution of 1% Blocker A solution spiked with synthetic Aβ1-40 peptide (1.56-100 pg/ml) except for non-transgenic mouse brain samples: In this case, the calibration curve was prepared in a correspondingly diluted APP knockout mouse forebrain spiked with synthetic Aβ1-40 peptide (1.56-100 pg/ml). For all samples and standards 25 μl were applied per well. For each determination duplicate wells were done. The mean values from the duplicate wells were used for calculations. The relative units for samples and standards were imported into SOFTmax PRO 4.0 for calculation of standard curves and quantification of samples.

### Determination of Formic Acid Soluble Aβ40 in APP23 Mouse Brain

Fifty microliter of forebrain homogenate was mixed with 116.6 μl 100% formic acid, resulting in a final formic acid concentration of 70%. Samples were stored on ice and vortexed every 5 minutes. For neutralization, 50 μl of the mixture were pipetted into a new tube, and 950 μl of 1 M Tris base, containing 1× Complete Protease inhibitor, was added. Tubes were stored at room temperature overnight and then centrifuged for 15 minutes at 14000 rpm in an Eppendorf Microzentrifuge at 4° C. From the top layer, 100 μl are removed and mixed with 100 μl of 3% Blocker A solution (part of the MesoScale assay kit). This sample was either directly applied to the assay plate (dilution 1:1332) or further diluted in 1% Blocker A solution.

### Analysis of Aβ40 in Mouse CSF

Mouse CSF samples (3 μl) was diluted with 57 μL 1% Blocker A (MSD) and 25 μl were applied to the assay plate.

### Analysis of Aβ40 in Mouse Plasma

Plasma samples (30 μl) were mixed with 30 μl of 3% Blocker A (MSD) and 25 μl were applied to the assay plate.

### Histological Analysis of Amyloid-Beta Plaques and Activated Astrocytes Using Double Fluorescence Immunohistochemistry

Amyloid plaques were stained using a rabbit anti-Aβ primary antibody which recognizes the C-terminal part of the amyloid peptide (the antibody was raised as described in Schrader-Fischer G, Paganetti P A, 1996; Schrader-Fischer G et al., 1997). Activated astrocytes were detected using a commercial rabbit anti-GFAP (reference Z0334 from Dako Schweiz GmbH, Baar, Switzerland).

All stainings were performed using the fully automated instrument Ventana Discovery® Ultra (Roche Diagnostics Schweiz A G, Rotkreuz, Switzerland). All chemicals were provided by Roche Diagnostic.

All study animals were used and brain tissue sections of 3 micrometers were freshly cut and collected on SuperFrost+ slides. The tissues sections were de-paraffinized and rehydrated under solvent-free conditions (EZprep solution) followed by antigen retrieval (demasking) performed by heat retrieval cycles for 32 min in an EDTA based buffer (CC1 solution). Subsequently, slides were blocked for 4 min using the DISCOVERY Inhibitor (reference 07017944001 (Roche)). The primary antibody diluted at 1/20'000 in antibody diluent was manually added on tissue sections and incubated for 1 h at room temperature. A short post-fixation (glutaraldehyde at 0.05%) was performed before applying the multimer UltraMap-anti Rabbit HRP ready to use antibody (reference 05269717001) for 16 min.

Detection was performed using the DISCOVERY FITC® following the manufacturer's recommendations. Slides were then heat denaturated at 92° C. for 20 min before a manual application of the second primary antibody (anti-GFAP diluted at 1/2'000) and incubated for 1 h. UltraMap-anti-Rabbit HRP antibody was used again for 20 min to detect GFAP in combination with the DISCOVERY Rhodamine kit (reference 07259883001).

The slides were washed and mounted using Prolong® Gold antifade reagent (reference P36931, ThermoFisher, Switzerland) and further scanned with the Hamamatsu slide scanner instrument (NanoZoomer 2.0 HT, scanning software NDP-Scan Vers. 2.5, Hamamatsu Photonics France, Swiss Office, Solothurn, Switzerland) at the 40× objective. The scanning settings were as follows: the exposure time with the DAPI filter was set at 57 ms as well as for the FITC filter. The exposure time for the TRITC filter (detection of Rhodamine) was set at 14.2 ms.

### Analysis of Amyloid-Beta Plaques and Activated Microglia Cells Using Double Fluorescence Immunohistochemistry

Amyloid plaques were stained using the same antibody and the microglia cells were detected using a rabbit anti-IBA1 antibody (reference 019-19741) from Wako Chemicals GmbH (Neuss, Germany) and diluted at 1/200 in antibody diluent. The staining protocol was exactly similar to the protocol for amyloid-beta plaques and astrocytes. The slides were scanned with the same settings.

### Image Analysis

For the quantitative plaque evaluation based on image analysis, a proprietary image analysis platform (ASTORIA, Automated Stored Image Analysis) was developed based on MS Visual Studio 2010 and many functions from Matrox MIL V9 libraries (Matrox Inc, Quebec, Canada).

For the beta-amyloid plaque and neuroinflammation analysis, the following sequence of steps was performed:
Slides were scanned with Hamamatsu Nanozoomer at 40× magnification. For each fluorescence labelling (DAPI, FITC and TRITC), a separate image was created
Manually outline ROIs (regions of interest) for defining cortex in brain sections for Aβ plaque assessment on the green FITC channel image, then use the resulting outline also for the other two channel images (copy resulting xml files)
Run the in-house developed ImageScope (V12.1.0.5029, Aperio Inc., USA) plug-in for creation and export of *.tif image tiles (at 10× magnification) for each of the 3 fluorescence channels

Image batch processing:
Obtain the combined true color image (DAPI, FITC, TRITC) for each section through accessing each individual fluorescence channel image
Segmentation of valid sample (within outlined ROI) from black unstained background
Apply adaptive thresholding technique for segmentation of objects in green channel image (FITC-labeled Aβ plaques)
Separation of touching objects for correct subsequent individual object analysis after elimination of too small debris showing signal in the green (FITC) channel
Segmentation of TRITC-labeled objects in red channel (specific for GFAP or Iba1 staining indicating astrocytes or microglia) through morphological tophat transformation and thresholding
Feature based object classification

4 object categories
Unspecific debris (too faint, too small objects) to be excluded
Small plaques (40 . . . 1000 pixels)
Medium plaques (1000 . . . 6500 pixels)
Large plaques (>6500 pixels)

Computation of several morphometric and densitometric features for valid plaques
Number of plaques
“Specific optical density” which has been described to reflect the amount of protein (antigen) concentration, based on measuring and using the staining intensity of an appropriate antibody in a non-linear way (Rahier et al., 1989; Ruifrok et al., 2001)
assessment of “plaque-associated GFAP or Iba1” based on the ratio of TRITC+ signal vs plaque area, “proximal GFAP or Iba1” based on ratio of TRITC+ signal within dilation ring around plaque

### Results

TABLE 24
Compound 1 levels in brain and blood

| | Time (after study | | Mean Amount |
|---|---|---|---|
| Dose (food pellet) | start) | Tissue/Fluid | (nM) ± SD |
| 0.03 g/kg | 2 months | Blood | 134 ± 49 |
| 0.3 g/kg | 2 months | Blood | 1802 ± 306 |
| 0.03 g/kg | 4 months | Blood | 406 ± 76 |
| 0.3 g/kg | 4 months | Blood | 2597 ± 346 |
| 0.03 g/kg | 6 months | Blood | 207 ± 28 |
| 0.3 g/kg | 6 months | Blood | 1861 ± 151 |
| 0.03 g/kg | 6 months | Brain | 556 ± 92 |
| 0.3 g/kg | 6 months | Brain | 6152 ± 1212 |

Blood concentrations of Compound 1 were determined after 2 and 4 months of dosing, and at the end of the study at 6 months. As shown in Table 24, there was constant exposure over the course of the study with acceptable variation between animals, 18% (8-36%) on average. Average Compound 1 blood concentration was 0.25±0.13 μM (mean±SD) for the 0.03 g/kg food dosing group, and 2.10±0.47 μM for the 0.3 g/kg dosing group, in good agreement with the 10-fold difference in compound dose. The exposure observed in this study roughly corresponded to a 5 and a 45 mg/kg daily oral dose of Compound 1. The brain/blood ratio, determined at the end of the experiment, was 2.7 for the 0.03 g/kg group, and 3.3 for the 0.3 g/kg group.

Biochemical Determination of APP Metabolites: Triton TX-100-Soluble APP Metabolites from Mouse Brain

Brain homogenates were extracted with 1% Triton X-100 in buffer and the resulting supernatant was considered to represent soluble forms of APP metabolites. In addition to Aβ40 and 42, we determined the N-terminal APP fragments sAPPα (direct cleavage product of α-secretase) and sAPP8 (Swe) (direct product of BACE1 cleavage). As shown in Table 25, soluble Aβ40 and 42 moderately (less than 2-fold) increase over the course of the study in the non-treated groups. Since no change in the APP expression and Aβ generation is known to happen during this age, it is assumed that the increased values in the vehicle group (18-20 mo old) arise from “leakage” out of the Aβ deposits (which increase several fold, see below). Also the values for the soluble APP metabolites sAPPα and β did not change significantly in the non-treated groups.

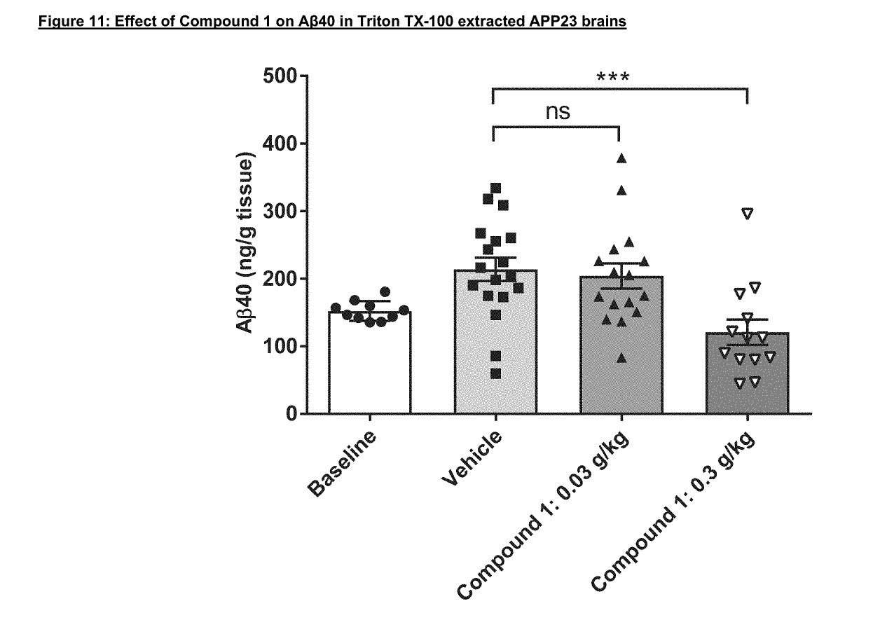

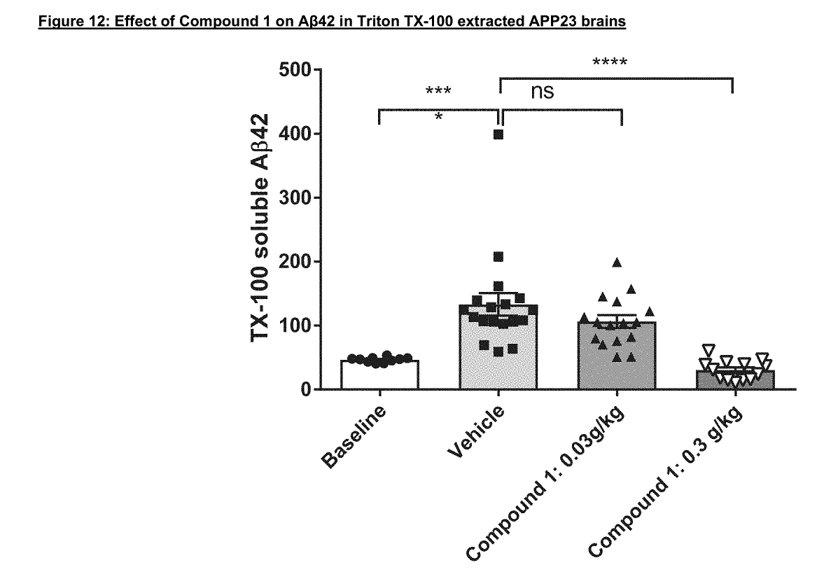

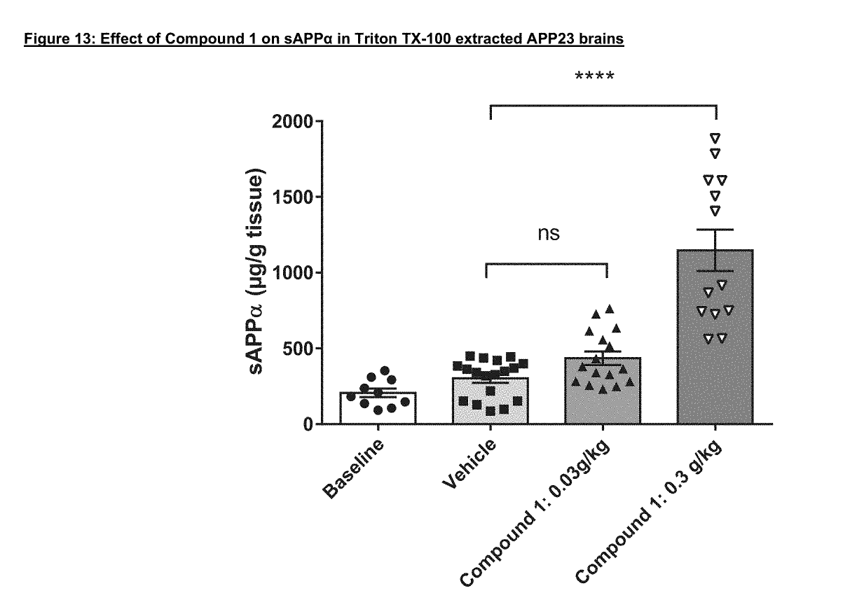

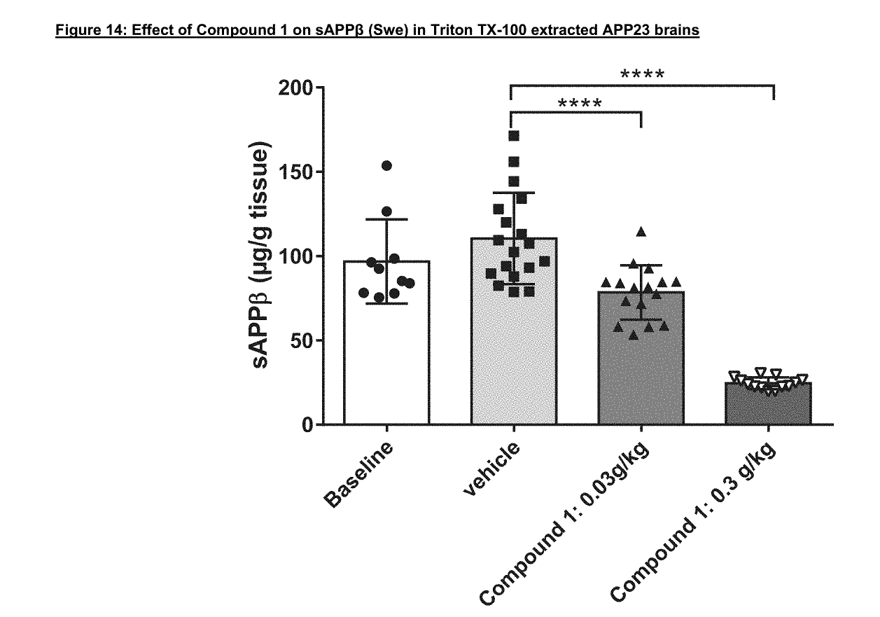

Mice treated with Compound 1 at the low dose (0.03 g Compound 1/kg food) showed a weak, but not significant reduction of soluble Aβ40 and 42 and a moderate increase in sAPPα (Tables 25 and 26, FIGS. 11, 12 and 13). Soluble APP (Swe) was significantly reduced by 29% (Tables 25 and 26, FIG. 14). Mice treated with the 0.3 g/kg dose of Compound 1 showed significant reduction of both Aβ and of sAPPβ (Swe), as well as a 3-fold increase in sAPPα (Tables 25 and 26, FIGS. 11, 12 and 14).

Taken together, Compound 1 treatment resulted in a dose-dependent reduction of all soluble BACE1 cleavage products and in a dose dependent increase in sAPPα.

TABLE 25
Mouse brain Aβ40 and 42 levels following treatment with Compound 1

| | | | | sAPPβ (Swe) |
|---|---|---|---|---|
| Treatment | Aβ40 (ng/g tiss.) | Aβ42 (ng/g tiss.) | sAPPα (μg/g tiss.) | (μg/g tiss.) |
| group (n) | Mean ± SEM | Mean ± SEM | Mean ± SEM | Mean ± SEM |
| Baseline (10) | 152.3 ± 4.6 | 46.4 ± 1.3 | 207 ± 28 | 96.8 ± 7.9 |
| 0.03 g/kg | 190.2 ± 18.6 | 106.3 ± 10.0 | 435 ± 40 | 78.5 ± 4.0 |
| food (16) | | | | |
| 0.3 g/kg food | 120.8 ± 18.9 | 30.3 ± 4.2 | 1147 ± 36 | 24.6 ± 0.96 |
| (13) | | | | |
| Vehicle (18) | 213.8 ± 17.3 | 133.0 ± 17.7 | 302 ± 30 | 110.5 ± 6.4 |

TABLE 26
Comparison of changes between groups (Dunnett's multiple comparison test)

| | | | | sAPPβ |
|---|---|---|---|---|
| Groups compared | Aβ40 | Aβ42 | sAPPα | (Swedish) |
| 0.03 g/kg vs | −4.6% | −20.1% | +44% | −29.0% |
| vehicle | not significant | not significant | not significant | p < 0.0001 |
| 0.3 g/kg vs vehicle | −43.7% | −77.3% | +279.8% | −77.7% |
| | p = 0.001 | p < 0.0001 | p < 0.0001 | p < 0.0001 |
| Vehicle vs | +141% | +186% | +31.4% | +12.4% |
| baseline | not significant | p < 0.0001 | not significant | not significant |

### APP Metabolites in CSF

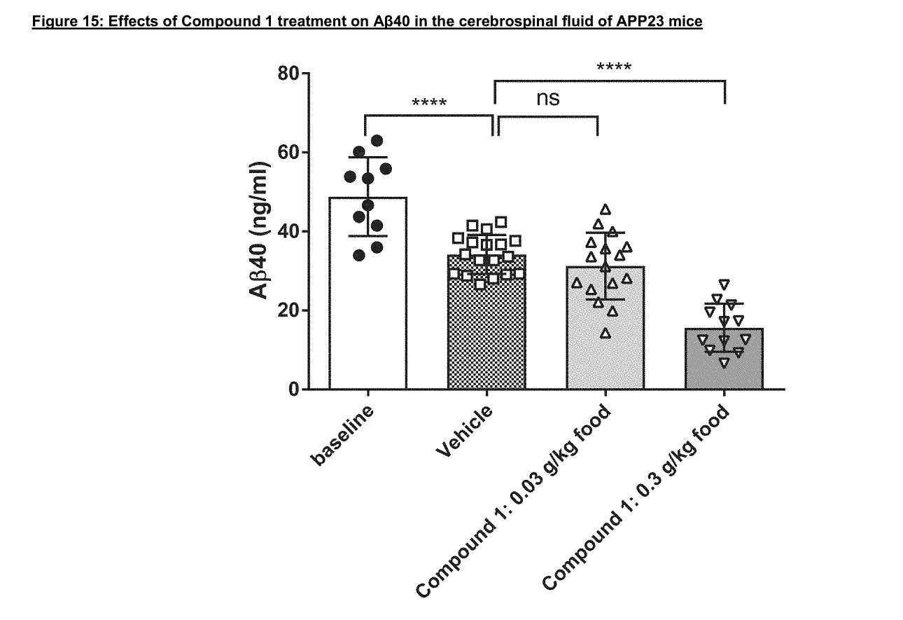

CSF was collected from all mice at necropsy. Samples from the baseline group were stored for approximately 6 months, and analyzed together with the rest of the samples at the end of the study. Data in Table 27 and FIG. 15 show that CSF Aβ are highest in the baseline group (APP23 mice at 12 months of age), but drop in the vehicle group (APP23 mice at 18 months of age). Compared to this vehicle group, CSF Aβ40 is non-significantly reduced in the 0.03 g/kg food Compound 1 treatment group, and significantly in the 0.3 g/kg food Compound 1 treatment group. The reason for the high baseline values is currently not known. It is hypothesized that this is an effect of long term storage, when dissociation of oligomeric forms of Aβ may lead to higher monomeric concentrations. CSF Aβ, more than Triton TX-100 solubilized Aβ from brain extracts, represents the steady-state concentration of soluble amyloid-β that directly responds to changes in Aβ generation. The small and non-significant treatment effects at the low Compound 1 dose (−4.6 to −20%), as well as the pronounced and significant effect at the high Compound 1 doses (−43.7 to −77%), are very comparable between the soluble Aβ species isolated from the brain tissue and Aβ40 in CSF.

TABLE 27
Summary of results for CSF Aβ40

| | Aβ40 (ng/ml) | Change vs | |
|---|---|---|---|
| Treatment group (n) | Mean ± SEM | vehicle | significance |
| Baseline (10) | 48.8 ± 3.15 | +42.7% | p < 0.0001 |
| 0.03 g/kg food (16) | 31.2 ± 2.11 | −8.6% | not significant |
| 0.3 g/kg food (12) | 15.6 ± 1.8 | −54.3% | p < 0.0001 |
| Vehicle (18) | 34.2 ± 1.16 | n.a. | n.a. |

### Formic Acid Soluble Amyloid-Beta Peptides in Forebrain

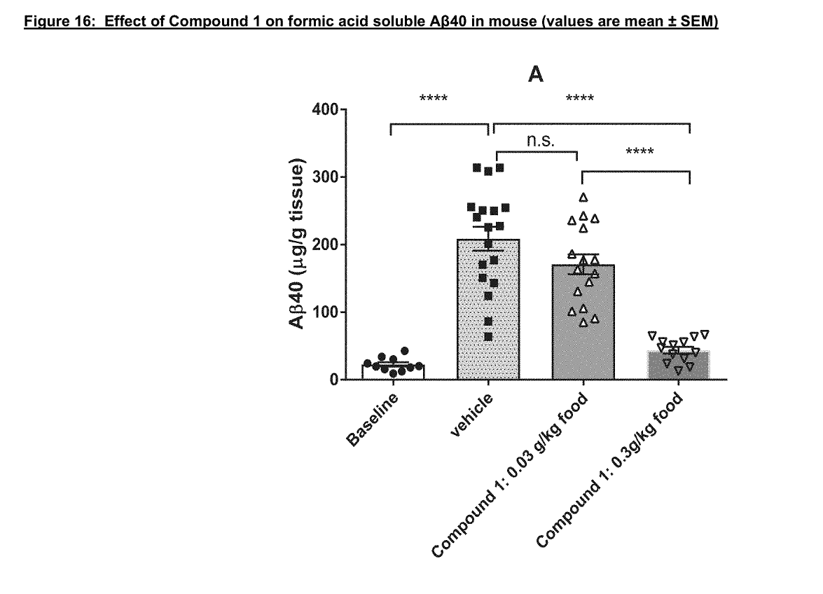

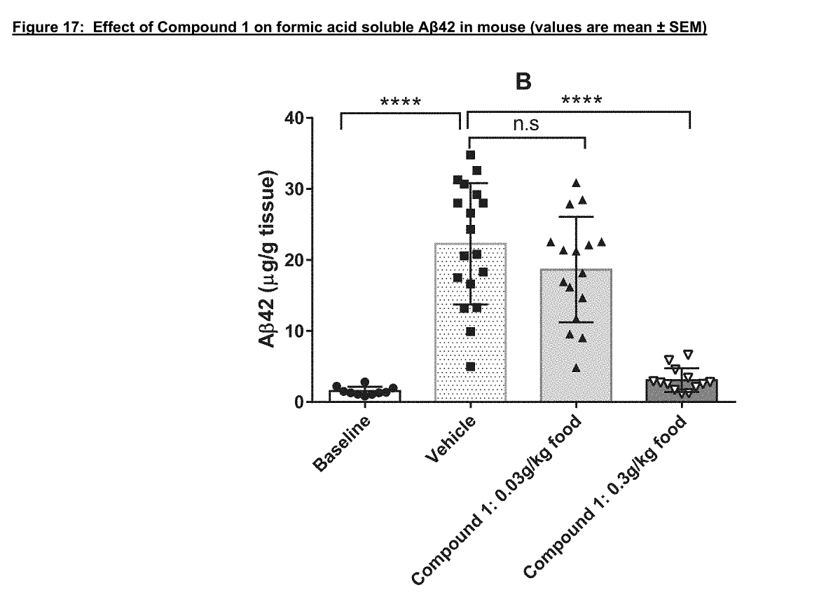

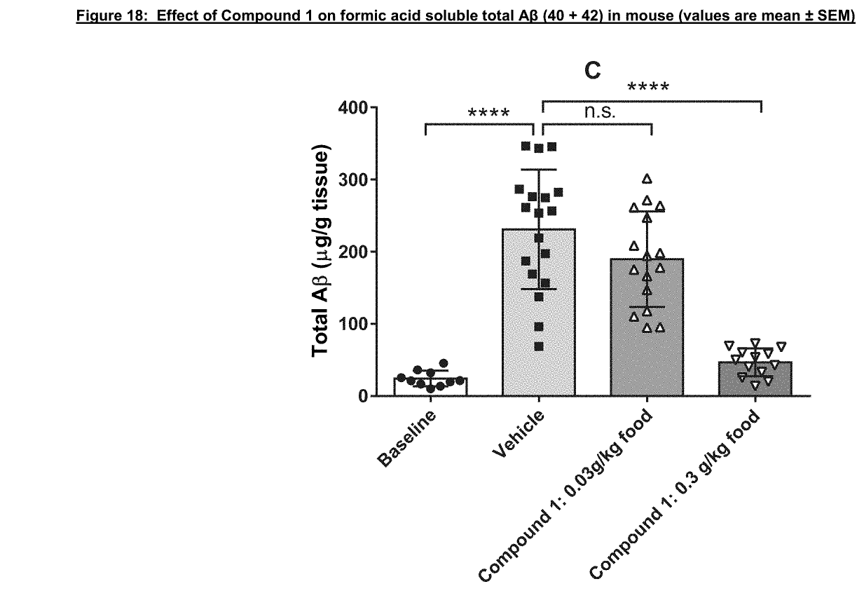

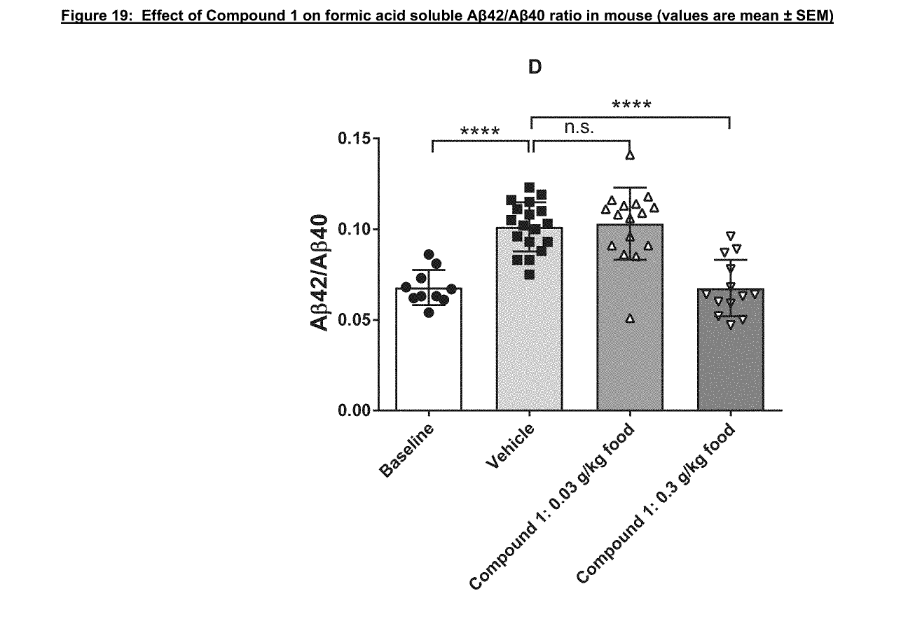

Treatment effects of Compound 1 on deposited forms of amyloid-β in the APP23 mouse brains were investigated after extraction of insoluble Aβ species with formic acid. As shown in Tables 28 and 29 and FIGS. 16 to 19, a massive increase of deposited Aβ was observed in the vehicle group, compared to baseline. Aβ42 increased more than Aβ40 (Aβ42/40 ratio increased by 55% in the vehicle group), in agreement with its higher aggregation propensity. Aβ40 and Aβ42 showed a reduction after treatment with the low dose of Compound 1 of around 17%, compared to vehicles, but it did not reach statistical significance. The Aβ42/40 ratio of the extracted material did not change. Strong and highly significant (around 80% vs vehicle) reduction of deposited Aβ40 and Aβ42 was observed in the high Compound 1 treatment group, and the Aβ42/40 ratio returned to baseline value of 0.07. In summary, the treatment with the high dose of Compound 1 almost completely blocked the increase of amyloid β in APP23 mice.

TABLE 28
Formic acid soluble amyloid-beta peptides in mouse forebrain

| Treatment | Aβ40 (μg/g) | Aβ42 (μg/g) | Total Aβ Mean ± | Ratio 42/40 |
|---|---|---|---|---|
| group (n) | Mean ± SEM | Mean ± SEM | SEM | Mean ± SEM |
| Baseline (10) | 22.7 ± 3.3 | 1.45 ± 0.2 | 24.2 ± 2.4 | 0.068 ± 0.0097 |
| 0.03 g/kg food | 170.9 ± 14.8 | 18.6 ± 1.9 | 189.5 ± 15.5 | 0.103 ± 0.0198 |
| (16) | | | | |
| 0.3 g/kg food | 43.7 ± 5.0 | 3.1 ± 0.5 | 46.8 ± 5.4 | 0.068 ± 0.0155 |
| (12) | | | | |
| Vehicle (18) | 208.7 ± 17.6 | 22.2 ± 2.0 | 231.0 ± 19.5 | 0.106 ± 0.0136 |

TABLE 29
Group comparison and statistics (Dunnett's multiple comparison test)

| Groups | | | | Aβ42/ |
|---|---|---|---|---|
| compared | Aβ40 | Aβ42 | Total Aβ | 40 ratio |
| 0.03 g/kg vs | −18.1% | −16.3% | −17.9% | −2.5% |
| vehicle | not | not significant | not | not |
| | | significant | significant | significant |
| 0.3 g/kg vs | −79.2% | −86.2% | −79.8% | −36.5% |
| vehicle | p < 0.0001 | p < 0.0001 | p < 0.0001 | p < 0.0001 |
| Vehicle vs | +820% | +1348% | +854% | +55% |
| baseline | p < 0.0001 | p < 0.0001 | p < 0.0001 | p < 0.0001 |

### Histological Assessment of Amyloid Pathology and Neuroinflammation: Plaque Numbers and Plaque Area

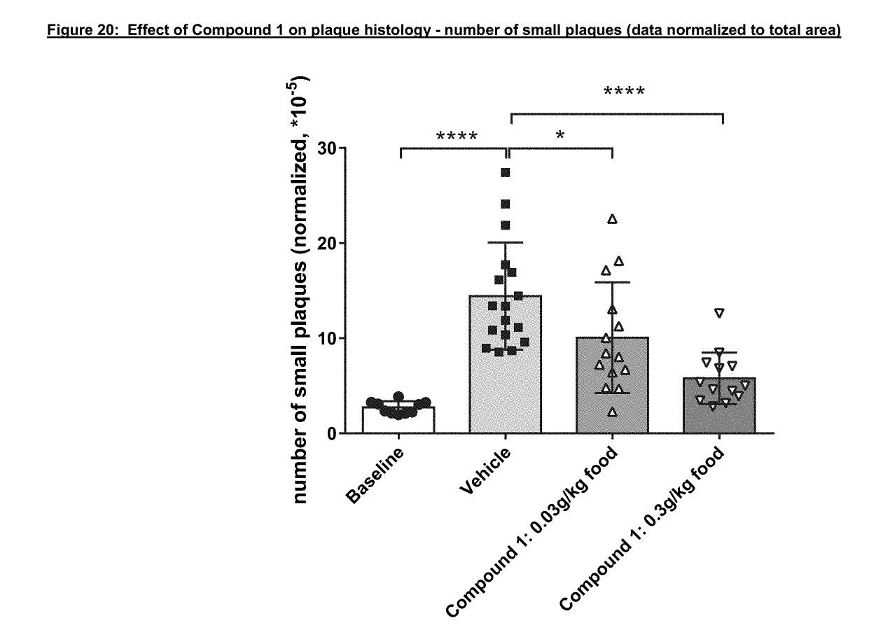

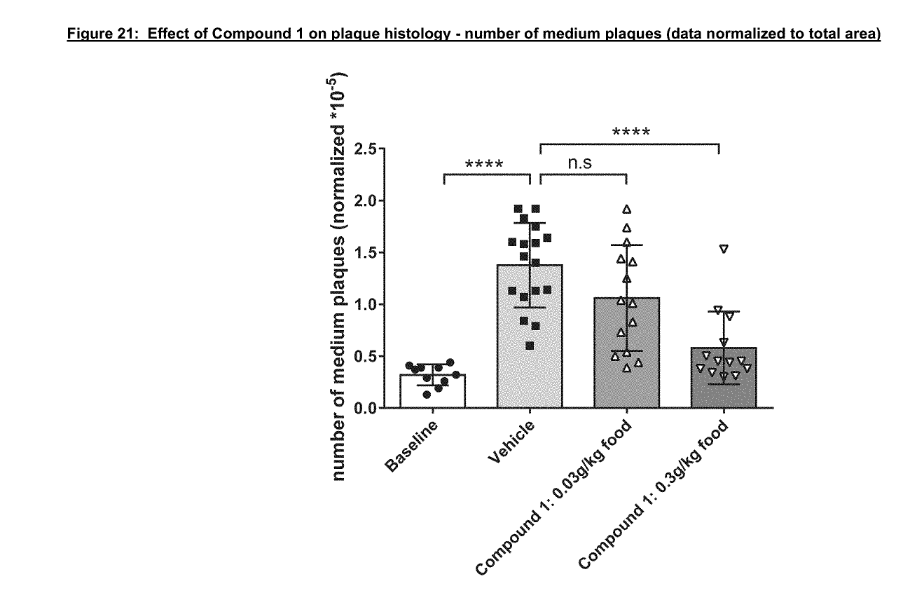

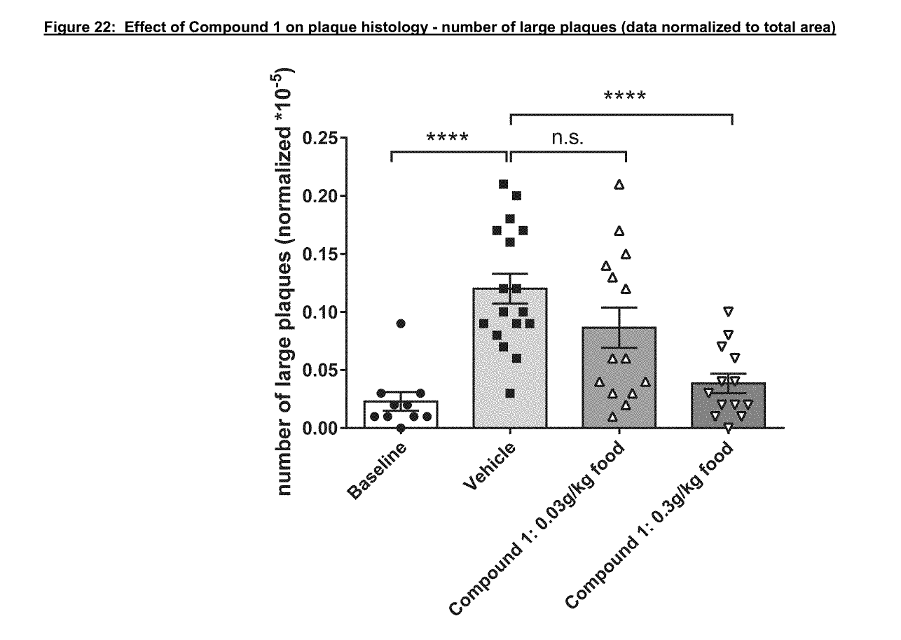

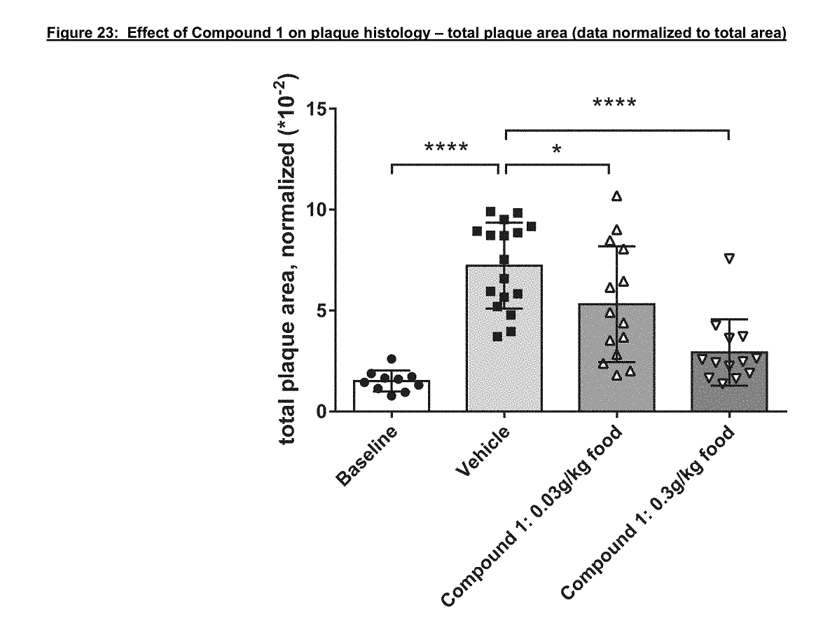

Amyloid plaques on APP23 brain slices were stained with an anti-Aβ antibody which recognizes the C-terminal part of the amyloid peptide. For a more detailed data analysis, the various forms of amyloid-β depositions in APP23 mice were classified into “small”, “medium” and “large” plaques. Furthermore, total immuno-stained area was determined. Quantification results are shown in Tables 30 and 31 and FIGS. 20 to 23. The majority of Aβ deposits were classified as “small” plaques, while the number of “medium” plaques was 10-fold and the number of “large” plaques was 100-fold lower. The numbers of all forms of plaques increase by approximately 4-6 fold in the vehicle group during the duration of the study, the same was observed for the total plaque area. Treatment with Compound 1 reduced the increase by about 25% in the low dose treatment group and about 60% in the high dose treatment group. The Aβ increase in the vehicle group and the effects in the 0.3 g/kg food Compound 1 treatment group are lower in the histological analysis, compared to the biochemical determination. The 2-dimensional histological analysis may not fully recapitulate the plaque volume changes that in reality occur in all 3 dimensions.

TABLE 30
Effect of Compound 1 treatment on plaque numbers and plaque area in APP23
mice, normalized to total area (1000000 * mean ± SEM)

| Treatment group | Number of | Number of | Number of | Total plaque |
|---|---|---|---|---|
| (n) | small plaques | medium plaques | large plaques | area |
| Baseline (10) | 2.73 ± 0.20 | 0.32 ± 0.03 | 0.02 ± 0.01 | 12.05 ± 1.37 |
| 0.03 g/kg food (13) | 10.06 ± 1.55 | 1.06 ± 0.14 | 0.09 ± 0.02 | 18.56 ± 2.59 |
| 0.3 g/kg food (12) | 5.78 ± 0.75 | 0.58 ± 0.10 | 0.04 ± 0.01 | 12.79 ± 2.33 |
| Vehicle (17) | 14.43 ± 1.37 | 1.38 ± 0.10 | 0.12 ± 0.01 | 22.33 ± 1.86 |

TABLE 31
Group comparison and statistics

| | Number of | Number of | Number of | Total plaque |
|---|---|---|---|---|
| Groups compared | small plaques | medium plaques | large plaques | area |
| 0.03 g/kg vs | −30.3% | −22.9% | −29.9% | −26.4% |
| vehicle | p < 0.05 | Not significant | Not significant | p < 0.05 |
| 0.3 g/kg vs vehicle | −60.0% | −57.9% | −68.2% | −59.5% |
| | p < 0.0001 | p < 0.0001 | p < 0.0001 | p < 0.0001 |
| Vehicle vs | 428.6% | 331.2% | 500% | 378.8% |
| baseline | p < 0.0001 | p < 0.0001 | p < 0.0001 | p < 0.0001 |

### Effects on Activated Astrocytes

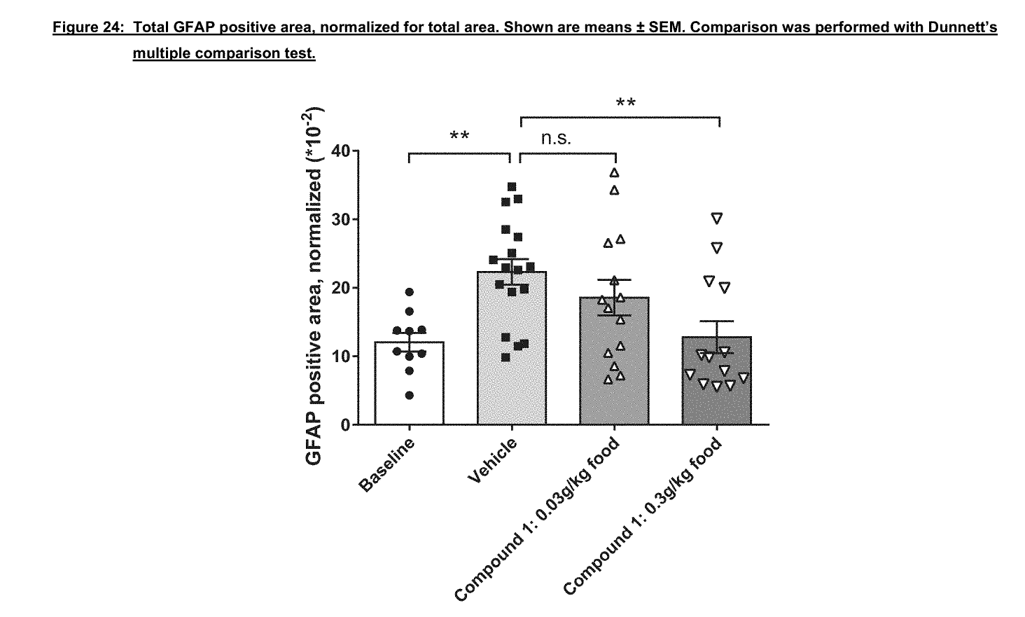

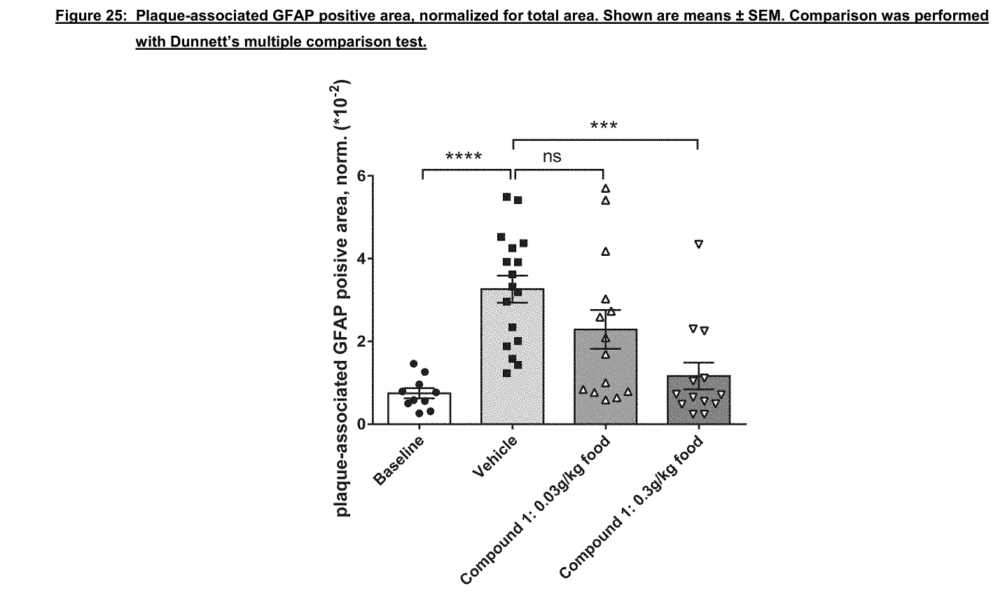

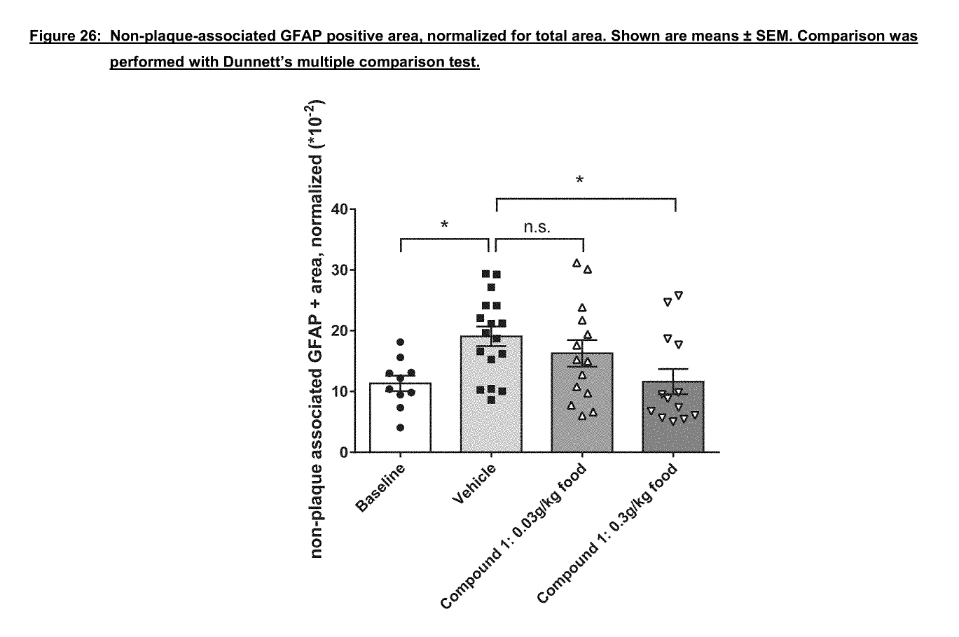

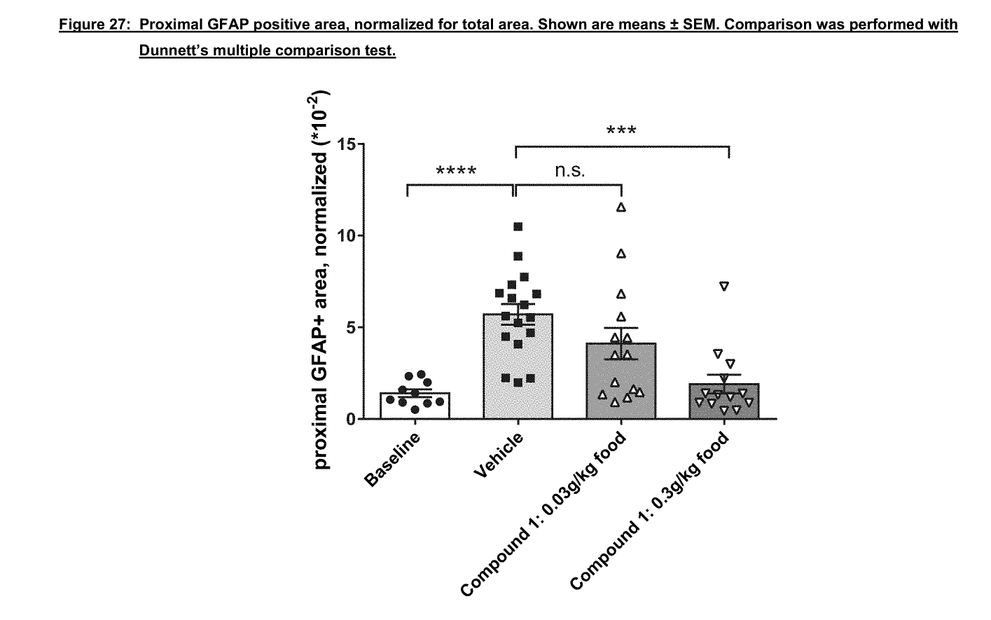

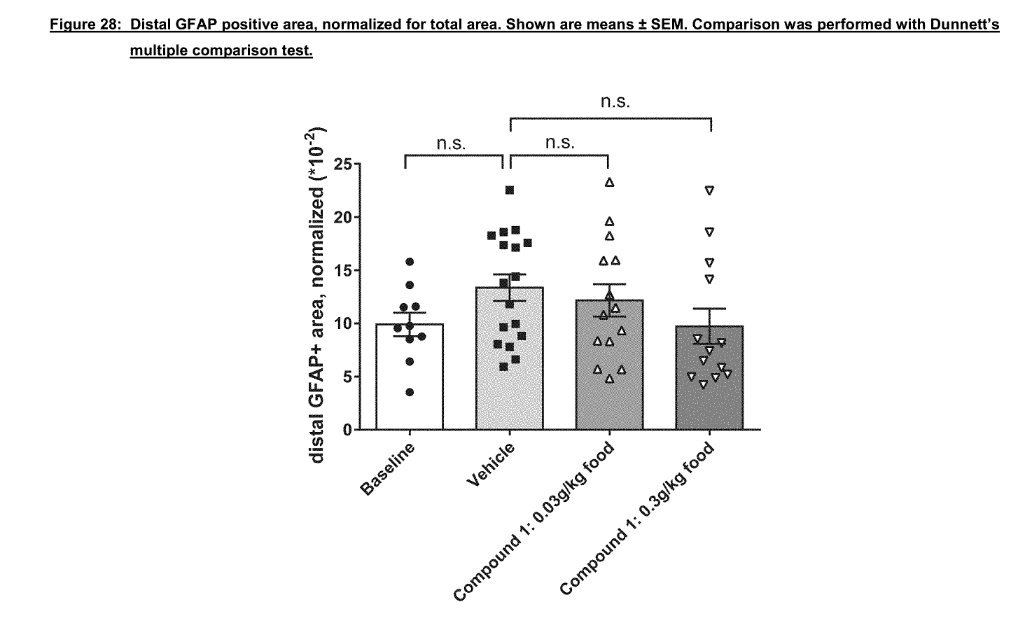

GFAP (Glial Acidic Fibrillary Protein) is found in resting as well as in activated astrocytes. GFAP immunoreactivity is often used as a marker of astrocyte number and activation. In APP23 mice, the normalized GFAP positive area increased with mouse age approximately 2-fold, and this increase was reduced by Compound 1 treatment in a dose-dependent manner (Tables 32 and 33 and FIGS. 24 to 28). GFAP immunoreactivity was further dissected with respect to association with amyloid plaques (performed identically to IBA1 immunoreactivity). This analysis shows that the vast majority of GFAP immunoreactivity is non-plaque associated (distal), and only 10% is plaque associated or proximal. The fraction of plaque-associated and proximal GFAP immuno-reactivity was increased in the vehicle group, suggesting a dominating increase in astrocyte number/activation in the close neighborhood of amyloid plaques. The increase with aging was low for distant and not plaque associated GFAP positive staining. The effect of Compound 1 treatment was also distinct between the plaque-associated and the non-plaque associated GFAP immunoreactivity: The effects on the plaque-associated/proximal GFAP immunoreactivity were stronger than on the non-plaque associated/distal staining. These data suggest that Compound 1 exerts its effect on GFAP staining primarily in the direct vicinity of amyloid plaques, most probably by way of an effect on the plaques themselves.

TABLE 32
Effect of Compound 1 treatment on activated astrocytes, expressed as GFAP
positive area, normalized to total area (100 * mean ± SEM)

| | | plaque- | non-plaque | | |
|---|---|---|---|---|---|
| | Total | associated | associated | | Distal |
| | GFAP+ | GFAP+ | GFAP+ | Proximal | GFAP+ |
| Treatment group (n) | area | area | area | GFAP+ area | area |
| Baseline (10) | 12.05 ± 1.37 | 0.74 ± 0.12 | 11.31 ± 1.28 | 1.41 ± 0.21 | 9.90 ± 1.11 |
| 0.03 g/kg food (13) | 18.56 ± 2.59 | 2.29 ± 0.47 | 16.27 ± 2.19 | 4.11 ± 0.86 | 12.16 ± 1.52 |
| 0.3 g/kg food (12) | 12.79 ± 2.33 | 1.16 ± 0.32 | 11.63 ± 2.06 | 1.91 ± 0.51 | 9.72 ± 1.65 |
| Vehicle (17) | 22.33 ± 1.86 | 3.26 ± 0.33 | 19.07 ± 1.62 | 5.71 ± 0.56 | 13.36 ± 1.25 |

TABLE 33
Treatment effects and statistics for normalized GFAP positive area

| | | plaque- | non-plaque | | |
|---|---|---|---|---|---|
| Groups | Total GFAP+ | associated | associated | Proximal | Distal |
| compared | area | GFAP+ area | GFAP+ area | GFAP+ area | GFAP+ area |
| 0.03 g/kg vs | −16.9% | −29.8% | −14.6% | −28.0% | −8.9% |
| vehicle | not | not | not | not | not |
| | significant | significant | significant | significant | significant |
| 0.3 g/kg vs | −42.7% | −64.3% | −39.0% | −66.6% | −27.2% |
| vehicle | p < 0.01 | p < 0.001 | p < 0.05 | p < 0.001 | not |
| | | | | | significant |
| Vehicle vs | +85.3% | +338.2% | +68.6% | +306.4% | +34.9% |
| baseline | p < 0.01 | p < 0.0001 | p < 0.05 | p < 0.0001 | not |
| | | | | | significant |

### Effects on IBA1 Positive Microglia

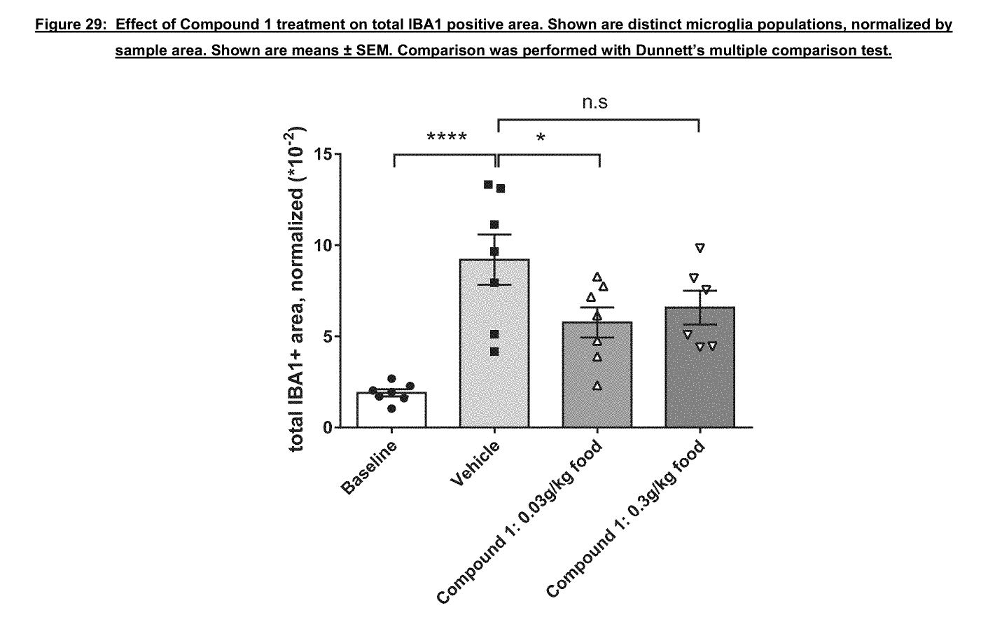

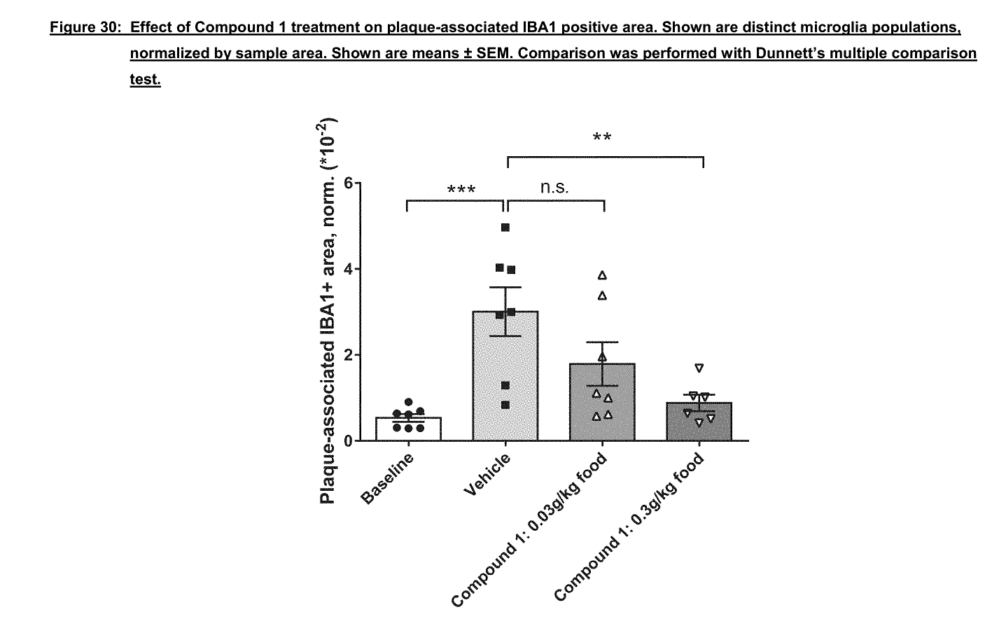

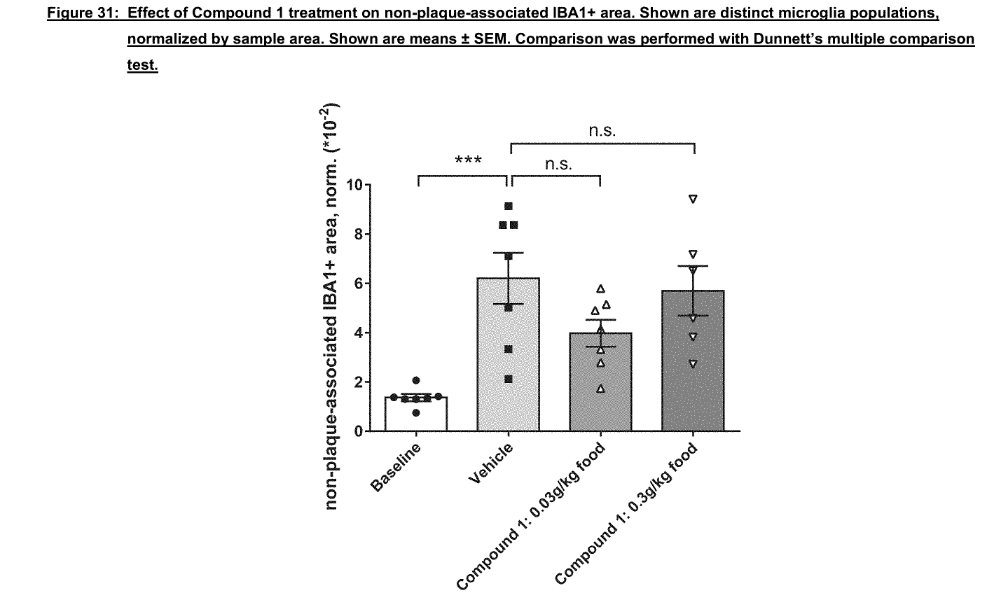

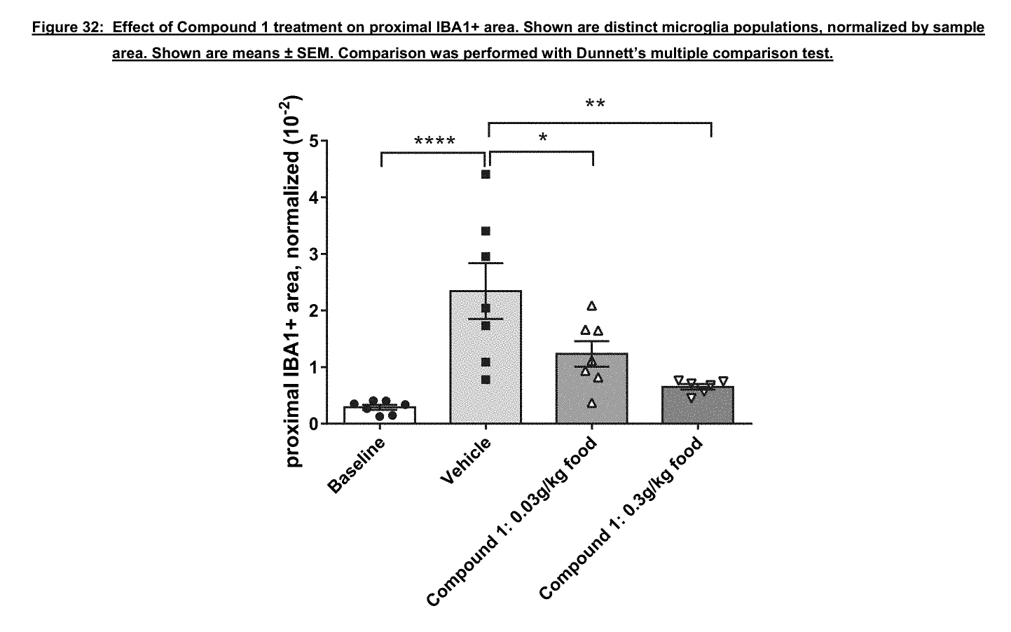

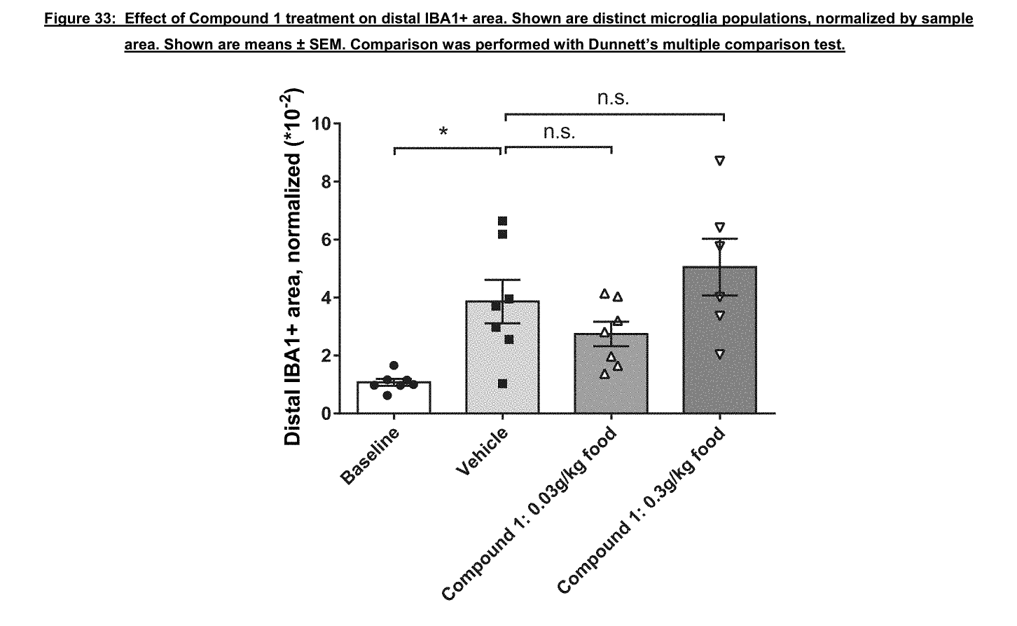

IBA1 (Ionized calcium binding adaptor molecule 1) is a microglia/macrophage specific protein. IBA1 immunoreactivity is often used as a marker of microglia number and activation. In APP23 mice, the normalized IBA1 positive area increased with mouse age approximately 5-fold, and this increase was reduced by Compound 1 treatment in a dose-dependent manner (Tables 34 and 35). IBA1 immunoreactivity was further dissected with respect to association with amyloid plaques. This analysis shows that approximately 75% of IBA1 immunoreactivity is non-plaque associated (distal), and only 25% is plaque associated or proximal. The fraction of plaque-associated and proximal IBA1 immuno-reactivity was increased in the vehicle group. To a lesser extent, also distant and not plaque associated IBA1 positive staining increased with mouse age. The effect of Compound 1 treatment was also distinct between the plaque-associated and the non-plaque associated IBA1 immunoreactivity: The effects on the plaque-associated/proximal IBA1 immunoreactivity were strong and significant. No significant effects were found on the non-plaque associated/distal staining. This is further illustrated in FIGS. 29 to 33, showing the effects of total IBA1 staining and plaque-associated IBA1 staining, relative to plaque area. These data suggest that Compound 1 exerts its effect on IBA1 staining primarily in direct vicinity of amyloid plaques, most probably via affecting the plaques themselves. Effects on plaques do not as strongly affect the microglia activation/number which is distant from the plaques (which is the majority of IBA1+immunoreactivity.

TABLE 34
Effect of Compound 1 treatment on IBA1-positive microglia, normalized by total
area (values are mean ± SEM)

| | | Plaque- | Non-plaque- | | |
|---|---|---|---|---|---|
| Treatment group | Total IBA1+ | associated | associated | Proximal | Distal IBA1+ |
| (n) | area | IBA1+ area | IBA1+ area* | IBA1+ area | area |
| Baseline (7) | 1.94 ± 0.20 | 0.53 ± 0.09 | 1.37 ± 0.15 | 0.29 ± 0.04 | 1.08 ± 0.12 |
| 0.03 g/kg food (7) | 5.76 ± 0.83 | 1.79 ± 0.51 | 3.98 ± 0.54 | 1.23 ± 0.22 | 2.74 ± 0.42 |
| 0.3 g/kg food (6) | 6.31 ± 0.92 | 0.88 ± 0.19 | 5.70 ± 1.00 | 0.65 ± 0.05 | 5.05 ± 0.98 |
| Vehicle (7) | 9.24 ± 1.38 | 3.00 ± 0.57 | 6.20 ± 1.04 | 2.34 ± 0.49 | 3.86 ± 0.75 |

TABLE 35
Treatment effects and statistics for normalized IBA1 positive area

| | | Plaque- | Non-plaque- | | |
|---|---|---|---|---|---|
| Groups | Total IBA1+ | associated | associated | Proximal | Distal IBA1+ |
| compared | area | IBA1+ area | IBA1+ area* | IBA1+ area | area |
| 0.03 g/kg vs | −37.4% | −40.5% | −39.2% | −47.4% | −28.9% |
| vehicle | p < 0.05 | not significant | not significant | p < 0.05 | not significant |
| 0.3 g/kg vs | −28.5% | −70.6% | −8.2% | −72.2% | +30.7% |
| vehicle | not significant | p < 0.01 | not significant | p < 0.01 | not significant |
| Vehicle vs | +374.6% | +389.3% | +360.9% | +598.4% | +287.1% |
| baseline | p < 0.001 | p < 0.001 | p < 0.001 | p < 0.0001 | p < 0.05 |

### Example 8: Summary of a Randomised, Double-Blind, Placebo-Controlled, Study to Evaluate the Efficacy of Compound 1 in Participants at Risk for the Onset of Clinical Symptoms of AD

In the clinical trial described herein, the identification of ApoE4 homozygotes is employed as a prognostic enrichment strategy to select individuals with a greater likelihood of having substantial worsening in cognition, in a reasonable timeframe, that can be practically assessed within the setting of a clinical trial. This study is listed in ClinicalTrials.gov under the NCT02565511 Identifier code. In the alternative, this example may be conducted with cognitively unimpaired ApoE4 carriers (homozygotes; or heterozygotes with additional enrichment for brain amyloid (“amyloid-positivity”) determined, for example, by PET or CSF measurement), aged 60 to 75 years, at a once daily oral dose of 15 or 50 mg Compound 1. This study is listed in ClinicalTrials.gov under the NCT03131453 Identifier code.

During the treatment duration of at least 5 years in the proposed clinical trial, it is expected that a significant proportion of the participants will be diagnosed with mild cognitive impairment (MCI) or dementia due to AD. The majority of the diagnoses are expected to be MCI, which is expected to precede diagnosis of dementia by 2-4 years.

TABLE 36
Summary of a randomised, double-blind, placebo-controlled, study to evaluate the
efficacy of Compound 1 in participants at risk for the onset of clinical symptoms of AD

| | |
|---|---|
| Title | A randomized, double-blind, placebo-controlled study to evaluate the |
| | efficacy of Compound 1 in participants at risk for the onset of clinical |
| | symptoms of Alzheimer's disease (AD). |
| Study type | Interventional. |
| Purpose and | The purpose of this study is to determine the effects of the therapy on |
| rationale | cognition, global clinical status, and underlying pathology in participants |
| | at risk for the onset of clinical symptoms of AD. Cognitively unimpaired |
| | individuals with APOE4 homozygote (HM) genotype and age 60 to |
| | 75 years, inclusive, are selected as they represent a population at |
| | particularly high risk of progression to Mild Cognitive impairment (MCI) |
| | due to AD and/or dementia due to AD. |
| Primary | To demonstrate the effects of Compound 1, vs. placebo on Time-to- |
| Objective(s) | event (TTE), with event defined as a diagnosis of MCI due to AD or |
| | dementia due to AD, whichever occurs first during the course of the |
| | study. |
| | To demonstrate the effects of Compound 1 vs. placebo on cognition |
| | as measured by the change from Baseline to Month 60 in the APCC |
| | (API Preclinical Composite Cognitive Battery) test score (Langbaum |
| | JB et al., 2014). |
| Secondary | Key secondary objective |
| Objectives | To demonstrate the effects of Compound 1, vs. placebo on global |
| | clinical status as measured by the change from Baseline to |
| | Month 60 in Clinical Dementia Rating Scale Sum of Boxes (CDR- |
| | SOB) score (Morris JC, 1993). |
| | Secondary objectives |
| | To demonstrate the safety and tolerability of Compound 1, vs. |
| | placebo as measured by adverse events (AEs), and changes in the |
| | brain structural MRI, laboratory tests, skin examinations, non- |
| | cognitive neurological and psychiatric findings including Columbia |
| | Suicide Severity Rating Scale (C-SSRS) (Posner K et al., 2011), vital |
| | signs and electrocardiogram (ECG). |
| | To demonstrate the effects of Compound 1, vs. placebo on |
| | cognition as measured by changes from Baseline to Month 60 on the |
| | Total Scale score and individual neurocognitive domain index scores |
| | of the Repeatable Battery for the Assessment of Neuropsychological |
| | Status (RBANS) (Randolph C. 1998). |
| | To demonstrate the effects of Compound 1, vs. placebo on function |
| | as measured by the change from Baseline to Month 60 in the |
| | Everyday Cognition scale (ECog) total scores reported by the |
| | participant and study partner, respectively (Farias ST et al., 2008). |
| | To demonstrate the effects of Compound 1, vs. placebo on AD- |
| | related biomarkers (amyloid deposition and measures of |
| | neurodegeneration) as measured by change from Baseline to |
| | Months 24 and 60 on: |
| | Binding of amyloid tracer ¹⁸F-florbetapir obtained using brain |
| | positron emission tomography (PET) imaging, |
| | Volumetric MRI measurements, and |
| | CSF Aβ₁₋₄₀, Aβ₁₋₄₂, total tau and phospho-tau₁₈₁ levels. |
| Study design | The Treatment Epoch follows a randomized, double-blind, placebo- |
| | controlled, design in which participants receive the investigational |
| | treatment or its matching placebo for at least 60 months up to a |
| | maximum of 96 months and no longer than when the target number of |
| | events for the TTE endpoint has been observed and confirmed. |
| Population | The Treatment Epoch population will consist of male and female |
| | participants at risk for the onset of clinical symptoms of AD, based on |
| | their APOE4 HM genotype and age (60 to 75 years of age). |
| | Randomization will be stratified by age group and region |
| Inclusion | Male or female, age 60 to 75 years inclusive. Females must be |
| criteria | considered post-menopausal and not of child bearing potential. |
| | Mini-Mental State Examination (MMSE) (Folstein MF et al., 1975) |
| | total score ≧24 and cognitively unimpaired as evaluated by memory |
| | tests performed at screening and as defined by: |
| | Homozygous APOE4 genotype, |
| | Participant's willingness to have a study partner |
| Exclusion | Current chronic treatment (>3 months) with strong CYP3A4 inducers |
| criteria | or strong CYP3A4 inhibitors. |
| | Any disability that may prevent the participants from completing all |
| | study requirements. |
| | Current medical or neurological condition that might impact cognition |
| | or performance on cognitive assessments. |
| | Advanced, severe progressive or unstable disease that may interfere |
| | with the safety, tolerability and study assessments, or put the |
| | participant at special risk. |
| | History of malignancy of any organ system, treated or untreated, |
| | within the past 60 months. |
| | Indication for, or current treatment with ChEls and/or another AD |
| | treatment (e.g. memantine). |
| | Brain MRI results showing findings unrelated to AD that, in the |
| | opinion of the Investigator might be a leading cause to cognitive |
| | decline, might pose a risk to the participant, or might prevent a |
| | satisfactory MRI assessment for safety monitoring. |
| | Suicidal Ideation in the past six months, or Suicidal Behavior in the |
| | past two years. |
| | A positive drug screen at Screening, if, in the Investigator's opinion, |
| | this is due to drug abuse. |
| | Significantly abnormal laboratory results at Screening, not as a result |
| | of a temporary condition. |
| | Current clinically significant ECG findings. |
| Investigational | Compound 1 and placebo: |
| and reference | Arm #1: Compound 1, 50 mg capsule p.o. for once daily administration |
| therapy | Arm #2: Placebo to Compound 1 p.o. |
| | Participants will be dispensed medication supplies for 3-month treatment |
| | with Compound 1, 50 mg or placebo for once daily, oral intake for the |
| | duration of the Treatment Epoch. |
| Efficacy | MCI due to AD or dementia due to AD (MCI/dementia) (diagnostic |
| assessments | verification form) |
| | API Preclinical Composite Cognitive (APCC) Battery |
| | Repeatable Battery for the Assessment of Neuropsychological Status |
| | (RBANS) |
| | Raven's Progressive Matrices (Raven JC at al., 1992; Raven JC, |
| | 2000) |
| | Mini Mental State Examination (MMSE) |
| | Clinical Dementia Rating Scale Sum of Boxes (CDR-SOB) |
| | Everyday Cognition Scale (ECog) |
| | Neuropsychiatric Inventory-Questionnaire (NPI-Q) (Kaufer et al., |
| | 2000) |
| | Geriatric Depression Scale (GDS) (Sheikh JI, Yesavage JA, 1986) |
| | Lifestyle questionnaire (Carlsson AC et al., 2013) |
| | Quality of Life (QOL-AD) (Logsdon RG et al., 1999 and Thorgrimsen |
| | L et al., 2003) |
| Safety | Physical and Neurological examination (including skin evaluation) |
| assessments | Vital signs |
| | Weight |
| | Laboratory evaluations |
| | Electrocardiogram (ECG) |
| | Safety brain MRI scans |
| | Adverse events and serious adverse events |
| | Columbia-Suicide Severity Rating Scale (C-SSRS) |
| Other | Pharmacokinetics |
| assessments | Biomarkers |
| | Imaging biomarkers |
| | Volumetric MRI |
| | Resting state functional MRI |
| | Amyloid PET |
| | FDG PET |
| | Fluid biomarkers |
| | CSF-based biomarkers |
| | Blood-based biomarkers (serum, plasma, blood for |
| | pharmacogenomics (RNA) and pharmacogenetics (DNA)) |
| Data analysis | The primary analysis comprises statistical tests of hypotheses of both |
| | primary endpoints. The statistical tests will compare active |
| | investigational treatment versus matching placebo as control group. A |
| | pre-defined testing strategy will be used to adjust the type I error rate for |
| | testing more than one hypothesis. |
| | Secondary endpoints (CDR-SOB, ECog, individual scales included in |
| | the APCC battery and RBANS, PET, Volumetric MRI, Total tau, |
| | phosphorylated tau in CSF) will all be analyzed using longitudinal |
| | models such as a generalized linear mixed model (GLMM) for the CDR- |
| | SOB and a mixed model repeat measure (MMRM) similar to the |
| | approach for the primary endpoint APCC with treatment as factor and |
| | adjusting for important covariates. For the secondary safety parameters |
| | (AEs, SAEs, laboratory results, vital signs, ECG, safety brain MRI |
| | scans) descriptive statistics will be provided. |

### Example 9: In Human Study of Pharmacokinetics of Compound 1 when Given Alone and in Combination with the Strong CYP3A4 Inhibitor Itraconazole or the Strong CYP3A4 Inducer Rifampicin

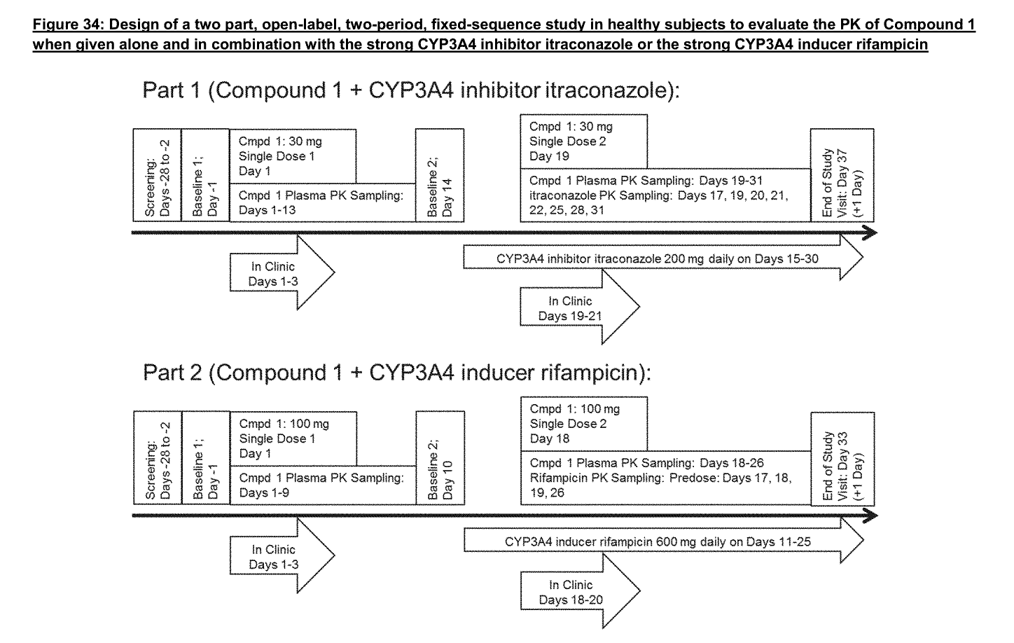

In a drug-drug interaction (DDI) study in healthy volunteers, the effect of a strong CYP3A4 inhibitor (itraconazole) and a strong CYP3A4 inducer (rifampicin) on the PK of Compound 1 was evaluated. The DDI study design is outlined in FIG. 34. Itraconazole, at a dose of 200 mg q.d., increased mean AUC of Compound 1 2-3-fold and mean Cmax of Compound 1 by 25%, when given together with Compound 1 as compared to when Compound 1 was given alone (Table 37). Rifampicin, at a dose of 600 mg q.d., decreased mean AUC of Compound 1 5-6-fold and mean Cmax of Compound 1 2.5-fold, when given together with Compound 1 as compared to when Compound 1 was given alone (Table 38). In conclusion, the effect of a strong CYP3A4 inducer and a strong CYP3A4 inhibitor on Compound 1 exposure in a Phase 1 study has shown that CYP3A4/5 is of major importance for the elimination of Compound 1.

TABLE 37
Pharmacokinetic results - Statistical analysis of the effect of itraconazole
on the plasma PK parameters of Compound 1: Compound 1 30 mg SD +
itraconazole 200 mg QD vs Compound 1 30 mg SD

| | | | Adjusted | Geometric mean | |
|---|---|---|---|---|---|
| Parameter | | | geometric | ratio | 90% CI for |
| [Unit] | Treatment | n* | mean | (Test/Reference) | ratio |
| AUCinf | Cmpd 1 30 mg SD | 17 | 3560 | 3.05 | [2.91, 3.20] |
| (ng * hr/mL) | Cmpd 1 30 mg SD + | 17 | 10900 | | |
| | Itraconazole 200 mg | | | | |
| | QD | | | | |
| AUClast | Cmpd 1 30 mg SD | 17 | 3150 | 2.20 | [2.11, 2.30] |
| (ng * hr/mL) | Cmpd 1 30 mg SD + | 17 | 6930 | | |
| | Itraconazole 200 mg | | | | |
| | QD | | | | |
| Cmax | Cmpd 1 30 mg SD | 17 | 74.1 | 1.23 | [1.18, 1.29] |
| (ng/mL) | Cmpd 1 30 mg SD + | 17 | 91.3 | | |
| | Itraconazole 200 mg | | | | |
| | QD | | | | |

n* = number of subjects with non-missing values.
An ANOVA model with fixed effects for treatment and subject was fitted to each log-transformed PK parameter. Results were back transformed to obtain ‘Adjusted geo-mean’, ‘Geo-mean ratio’ and ‘90% CI’.

TABLE 38
Pharmacokinetic results - statistical analysis of the effect of rifampicin on the plasma
PK parameters of Compound 1: Compound 1 100 mg SD + rifampicin 600 mg QD vs
Compound 1 100 mg SD

| | | | Adjusted | Geometric mean | |
|---|---|---|---|---|---|
| Parameter | | | geometric | ratio | |
| [Unit] | Treatment | n* | mean | (Test/Reference) | 90% CI for ratio |
| AUCinf | Cmpd 1 100 mg SD | 13 | 10200 | 0.172 | [0.152, 0.194] |
| (ng * hr/mL) | Cmpd 1 100 mg SD + | 13 | 1750 | | |
| | Rifampicin 600 mg QD | | | | |
| AUClast | Cmpd 1 100 mg SD | 13 | 8560 | 0.196 | [0.176, 0.219] |
| (ng * hr/mL) | Cmpd 1 100 mg SD + | 13 | 1680 | | |
| | Rifampicin 600 mg QD | | | | |
| Cmax | Cmpd 1 100 mg SD | 13 | 222 | 0.414 | [0.365, 0.470] |
| (ng/mL) | Cmpd 1 100 mg SD + | 13 | 92.2 | | |
| | Rifampicin 600 mg QD | | | | |

n* = number of subjects with non-missing values.
An ANOVA model with fixed effects for treatment and subject was fitted to each log-transformed PK parameter. Results were back transformed to obtain ‘Adjusted geo-mean’, ‘Geo-mean ratio’ and ‘90% CI’.

### Example 10: Evaluation of Change in A1342/A1340 Ratio from Baseline in Healthy Elderly ApoE4 Heterozygotes and ApoE4 Non-Carriers in Response to Treatment with Compound 1

Increased Aβ deposition in the brain can be determined by PET imaging of cortical Aβ using an established PET tracer, for example ¹¹C-Pittsburgh compound B, ¹⁸F-florbetaben, or ¹⁸F-flutemetamol, and also as a decrease in CSF Aβ1-42. Several studies have shown high concordance between PET imaging vs CSF Aβ1-42 analysis for the detection of amyloid-β pathology in the brain (Weigand S D et al., 2011; Barthel H et al., 2011; Schipke C G et al., 2017). The correlation suggests that the reduction in CSF Aβ1-42 is a result of increased amyloid-β deposition in the brain. In contrast to Aβ1-42, the CSF concentration of Aβ1-40, which is less prone to accumulate in cortical amyloid deposits, remains practically constant, even in patients with high cortical amyloid-β load. In agreement with this, it has been demonstrated that a more robust PET-CSF correlation is obtained when the CSF Aβ1-42/Aβ1-40 ratio is used (Pannee J et al., 2016; Janelidze S et al., 2016), instead of Aβ1-42 alone. While the use of the Aβ1-42/Aβ1-40 ratio as a diagnostic tool for the detection of amyloid-β pathology in the brain is well established, a change of this parameter in response to treatment with an anti-amyloid agent has not previously been described.

In the completed 3-month dose-ranging safety and tolerability clinical study in healthy elderly subjects described in Example 5, Aβ40 and Aβ42 concentrations in CSF were obtained by means of lumbar punctures before the first dose (baseline) and after 3 months of multiple dosing. It was found that a significant number of subjects have a baseline CSF Aβ42/Aβ40 below normal (below a cut-off of 0.09) indicative of cortical amyloid deposition. The percentage of subjects with a ratio below 0.09 is higher in the group of ApoE4 carriers (33%), compared to the non-carriers (15%). This is in agreement with the enhanced risk of ApoE4 carriers developing amyloidosis.

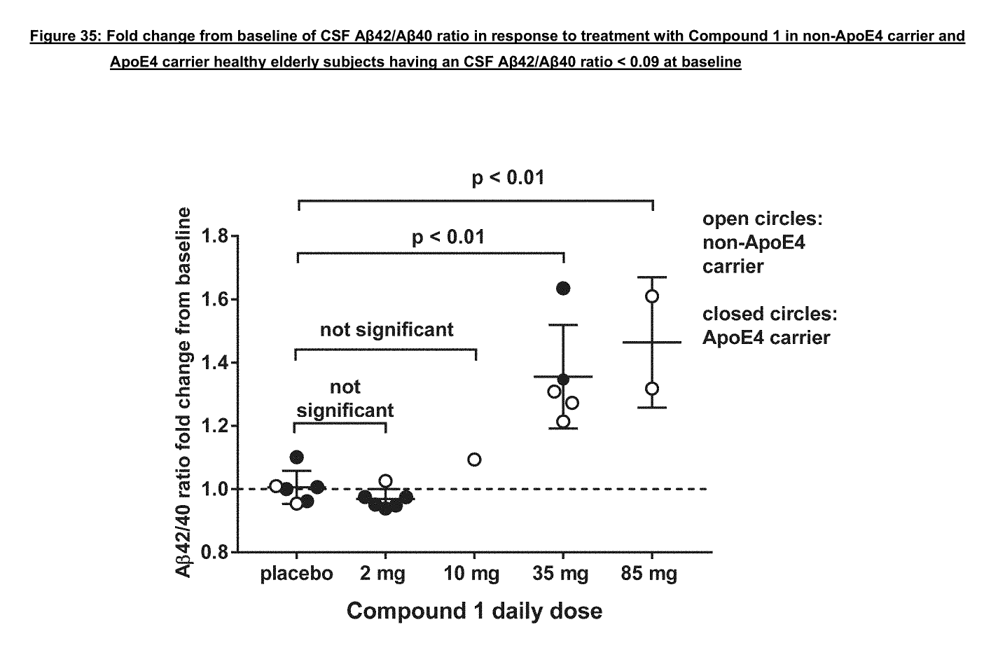

The CSF Aβ42/Aβ40 ratio at the end of a 3-month treatment with Compound 1 was determined in subjects having a baseline CSF Aβ42/Aβ40 ratio below 0.09, FIG. 35. It was found that treatment with Compound 1, in a dose-dependent manner, increased the Aβ42/Aβ40 ratio, compared with baseline value in the same subject. An increased Aβ42/Aβ40 ratio was found in response to treatment for both carriers and non-carriers of the ApoE4 allele. Specifically, the 35 mg and 85 mg daily doses resulted in a 1.36 (p<0.01 vs. placebo) and 1.46 (p<0.01 vs. placebo) fold increase of the Aβ42/Aβ40 ratio.

This result is indicative of increased transport of Aβ42 from the brain to CSF, corresponding to a reduced cortical amyloid-β load in subjects treated with the higher doses of Compound 1. The reduction in cortical amyloid-β load demonstrates that Compound 1 is capable of modifying the amyloid pathology characteristic of AD in both carriers and non-carriers of the ApoE4 allele and, therefore, is expected to be effective in the prevention of AD in either of these patient groups.

### Example 11: Pharmaceutical Compositions Comprising Compound 1

The pharmaceutical composition used in the clinical studies described in Examples 4 and 5 was formulated as a hard gelatin capsule (e.g. Capsugel, size 3) comprising the ingredients shown in Table 39 and prepared as described below.

TABLE 39
Manufacturing of 1 mg, 10 mg and 75 mg hard gelatin capsules
of Compound 1

| | Amount per batch (kg) | | |
|---|---|---|---|
| Ingredient | 1 mg | 10 mg | 75 mg |
| Batch size | 7500 units | 16,000 units | 7,100 units |
| Capsule fill | | | |
| Compound 1¹ | 0.0075 | 0.1600 | 0.5325 |
| Mannitol | 0.8568 | 1.7238 | 0.4242 |
| Pregelatinised starch | 0.2775 | 0.5520 | 0.1243 |
| Low-substituted | 0.0660 | 0.1408 | 0.0625 |
| Hydroxpropyl | | | |
| cellulose | | | |
| Hydroxypropylcellulose | 0.0432 | 0.0922 | 0.0409 |
| Sodium stearyl | 0.0180 | 0.0384 | 0.0170 |
| fumarate | | | |
| Talc | 0.0060 | 0.0128 | 0.0057 |
| Purified water² | q.s. | q.s. | q.s. |
| Weight capsule fill | 1.2750 | 2.7200 | 1.2071 |
| mix | | | |
| Empty capsule shell | | | |
| Capsule shell | 0.3600 | 0.7680 | 0.3408 |
| (theoretical weight) | | | |
| Total batch weight | 1.6350 | 3.4880 | 1.5479 |

¹Corresponding to a corrected drug substance content (= cc) of 100%. A compensation of drug substance is performed if the corrected drug substance content is ≦99.5%. The difference in weight is adjusted with Mannitol. The cc is calculated as given below: 100% − total related substances % − (residual solvents + water)%
²Removed during processing

Other batch sizes or dosage strengths may be prepared depending on clinical requirements and/or available equipment. The weight of individual components for other batch sizes corresponds proportionally to the stated composition.

### Description of Manufacturing Process of Compound 1: 1 mg and 10 mg Hard Gelatin Capsules

1. Blend Compound 1 drug substance and portion of mannitol.
2. Sieve the mixture of step 1.
3. Blend the mixture of step 2.
4. Sieve portion of mannitol and add to the mixture of step 3.
5. Blend the mixture of step 4.
6. Sieve remaining portion of mannitol, pre-gelatinised starch, low-substituted hydroxypropyl cellulose and hydroxypropyl cellulose. Add the sieved ingredients to the mixture of step 5.
7. Blend the mixture of step 6.
8. Sieve the blend of step 7.
9. Blend the mixture of step 8.
10. Dissolve hydroxypropyl cellulose in purified water under stirring to form binder solution. Add binder solution to the blend of step 9 and knead/granulate the mass.
11. Perform wet screening of mass from step 10 if necessary.
12. Dry the wet granules of step 11.
13. Screen the dried granules of step 12.
14. Sieve mannitol, low-substituted hydroxypropyl cellulose and talc and add to the sieved granules of step 13.
15. Blend the mixture of step 14.
16. Sieve sodium stearyl fumarate and add to mixture of step 15.
17. Blend the mixture of step 16 to get final blend.
18. Encapsulate the final blend from step 17.

### Description of Manufacturing Process of Compound 1: 75 mg Hard Gelatin Capsules

1. Sieve Compound 1 drug substance, mannitol, pre-gelatinised starch, low substituted
hydroxypropyl cellulose, hydroxypropyl cellulose.
2. Blend the sieved materials of step 1.
3. Sieve the mixture of step 2.
4. Blend the mixture of step 3.
5. Dissolve hydroxypropyl cellulose in purified water under stirring to form binder solution. Add binder solution to the blend of step 4 and granulate the mass.
6. Perform wet screening if necessary
7. Dry the wet granules of step 6.
8. Screen the dried granules of step 7.
9. Sieve mannitol, low-substituted hydroxypropyl cellulose and talc and add to sieved granules of step 8.
10. Blend the mixture of step 9.
11. Sieve sodium stearyl fumarate and add to step 10.
12. Blend the mixture of step 11 to get final blend.
13. Encapsulate the final blend of step 12.

The processes described above may be reasonably adjusted, while maintaining the same basic production steps, to compensate for different batch sizes, dosage strengths, and/or equipment characteristics. Granulation steps may be divided in sub-batches to utilise the qualified range of the equipment.

### REFERENCES

Albert M S et al., (2011) The diagnosis of mild cognitive impairment due to Alzheimer's disease: recommendations from the national Institute on Aging—Alzheimer's Association workgroups on diagnostic guidelines for Alzheimer's disease. Alzheimers Dement; 7:270-9.
Barthel H et al., (2011) Cerebral amyloid-beta PET with florbetaben (18F) in patients with Alzheimer's disease and healthy controls: a multicentre phase 2 diagnostic study. Lancet Neurol., 10: 424-435.
Carlsson A C et al., (2013) Seven modifiable lifestyle factors predict reduced risk for ischemic cardiovascular disease and all-cause mortality regardless of body mass index: A cohort study. International Journal of Cardiology; 168: 946-952.
Castellano J M et al., (2011) Human apoE Isoforms Differentially Regulate Brain Amyloid-b Peptide Clearance. Sci Transl Med; 3 (89): 89ra57).
Corder E H et al., (1993) Gene dose of apolipoprotein E type 4 allele and the risk of alzheimer's disease in late onset families. Science; 261(5123):921-3.
Cummings J L et al., (2014) Alzheimer's disease drug-development pipeline: few candidates, frequent failures. Alzheimer's Research & Therapy; 6:37.
Farias S T et al., (2008) The measurement of everyday cognition (ECog): Scale development and psychometric properties. Neuropsychology; 22(4):531-544.
Folstein M F et al., (1975) A practical method for grading the cognitive state of patients for the clinician. J. Psychiat. Res.; 12:189-198.
Forlenza O V et al., (2015) Cerebrospinal fluid biomarkers in Alzheimer's disease: Diagnostic accuracy and prediction of dementia. Alzheimers Dement. (Amst); 1(4):455-463.
Foster N L et al., (2007) FDG-PET Improves Accuracy in Distinguishing Frontotemporal Dementia and Alzheimer's Disease. Brain; 130(10):2616-2635.
Genin E et al., (2011) ApoE and Alzheimer disease: A major gene with semi-dominant inheritance. Mol. Psychiatry; 16(9):903-907.
Harada R et al., (2016) 18F-THK5351: A Novel PET Radiotracer for Imaging Neurofibrillary Pathology in Alzheimer Disease. J. Nucl. Med.; 57(2):208-214.
Head E et al., (2012) Alzheimer's Disease in Down Syndrome. Eur. J. Neurodegener. Dis.; 1(3):353-364.
Herskovitz A Z et al., (2013) A Luminex assay detects amyloid β oligomers in Alzheimer's disease cerebrospinal fluid. PLoS ONE; 8(7):e67898. doi:10.1371/journal.pone.0067898.
Janelidze S et al., (2016) Swedish BioFINDER study group, Hansson O.CSF Aβ42/Aβ40 and Aβ42/A838 ratios: better diagnostic markers of Alzheimer disease. Ann Clin Transl Neurol. 3(3):154-65.
Jansen W J et al., (2015) Prevalence of cerebral amyloid pathology in persons without dementia: a meta-analysis JAMA; 313(19):1924-38.
Kaufer et al., (200)) Validation of the NPI-Q, a brief clinical form of the Neuropsychiatric Inventory. J. Neuropsychiatry Clin. Neuroscience; 12: 233-239
Knouff C et al. (1999) Apo E structure determines VLDL clearance and atherosclerosis risk in mice. J. Clin. Invest.; 103 (11): 1579-1586).
Kramp V P, Herrling P, (2011) List of drugs in development for neurodegenerative diseases: Update June 2010. Neurodegenerative Dis.; 8:44-94.
Langbaum J B et al., (2014) An empirically derived composite cognitive test score with improved power to track and evaluate treatments for preclinical Alzheimer's disease. Alzheimers Dement.; 10(6):666-74.
Liu C C et al., (2013) Apolipoprotein E and Alzheimer disease: risk, mechanisms and therapy. Nat. Rev. Neurol.; 9:106-118.
Logsdon R G et al., (1999) Quality of life in Alzheimer's disease: Patient and caregiver reports. Journal of Mental Health & Aging; 5(1):21-32.
Luo G et al., (2004) CYP3A4 Induction by Xenobiotics: Biochemistry, Experimental Methods and Impact on Drug Discovery and Development. Current Drug Metabolism; 5:483-505
Mattsson N et al., (2015) Predicting Reduction of Cerebrospinal Fluid β-Amyloid 42 in Cognitively Healthy Controls. JAMA Neurology; 72(5):554-560.
McKhann G M et al., (2011) The diagnosis of dementia due to Alzheimer's disease: Recommendations from the National Institute of Aging-Alzheimer's Association workgroups on diagnostic guidelines for Alzheimer's disease. Alzheimer's and Dementia; 7:263-269.
Medina M et al., (2014) New perspectives on the role of tau in Alzheimer's disease. Implications for therapy. Biochem. Pharmacol.; 88(4):540-547.
Morris J C, (1993) The Clinical Dementia Rating (CDR): Current version and scoring rules. Neurology; 43:2412-2414.
Neumann U. et al., (2015) A novel BACE inhibitor NB-360 shows a superior pharmacological profile and robust reduction of amyloid-β and neuroinflammation in APP transgenic mice. Molecular Neurodegeneration; 10:44.
O'Brien R J, Wong P C, (2011) Amyloid Precursor Protein Processing and Alzheimer's disease. Annu. Rev. Neurosci.; 34:185-204.
Palmqvist S et al., (2016) Cerebrospinal fluid analysis detects cerebral amyloid-ρ accumulation earlier than positron emission tomography. Brain; 139:1226-1236.
Pannee J et al., (2016) Reference measurement procedure for CSF amyloid beta (Aβ)1-42 and the CSF Aβ1-42/Aβ1-40 ratio—a cross-validation study against amyloid PET. J Neurochem; 139(4):651-658.
Posner K et al., (2011) The Columbia-Suicide Severity Rating Scale: Initial Validity and Internal Consistency Findings From Three Multisite Studies With Adolescents and Adults. Am. J. Psychiatry; 168:1266-1277.
Rahier J et al., (1989) Determination of antigen concentration in tissue sections by immunodensitometry. Laboratory Investigations; 61:357-363.
Randolph C, (1998) The Repeatable Battery for the Assessment of Neuropsychological Status (RBANS). Pearson; San Antonio.
Raven J C et al., (1992) Standard progressive matrices-1992 edition; Raven manual: Section 3. Oxford Psychologists Press; Oxford.
Raven J C, (2000) Standard Progressive Matrices-1998 Edition, updated 2000. Manual for Standard Progressive Matrices (Section 3): NCS Person, Inc.; San Antonio.
Ruifrok A C et al., (2001) Quantification of histochemical staining by color deconvolution. Anal Quant. Cytol. Histol.; 23:291-299.
Schipke C G et al., (2017) Correlation of florbetaben PET imaging and the amyloid peptide Aβ42 in cerebrospinal fluid. Psychiatry Research (265):98-101.
Schrader-Fischer G, Paganetti P A, (1996) Effect of alkalizing agents on the processing of the β-amyloid precursor protein. Brain Research; 716(1-2):91-100.
Schrader-Fischer G et al., (1997) Insertion of Lysosomal Targeting Sequences to the Amyloid Precursor Protein Reduces Secretion of βA4. Journal of Neurochemistry 68(4):1571-1580.
Schreiber S et al., (2015) Comparison of Visual and Quantitative Florbetapir F 18 Positron Emission Tomography Analysis in Predicting Mild Cognitive Impairment Outcomes. JAMA Neurol.; 72(10):1183-1190.
Sevrioukova I F, Poulos T L, (2015) Current Approaches for Investigating and Predicting Cytochrome P450 3A4-Ligand Interactions. Adv. Exp. Med. Biol.; 851:83-105.
Sheikh J I, Yesavage J A, (1986) Geriatric Depression Scale (GDS). Recent evidence and development of a shorter version. In T. L. Brink (Ed.), Clinical Gerontology: A Guide to Assessment and Intervention (pp. 165-173). N Y: The Haworth Press, Inc.
Shimshek D R et al., (2016) Pharmacological BACE1 and BACE2 inhibition induces hair depigmentation by inhibiting PMEL17 processing in mice. Sci. Rep.; 6:21917; doi: 10.1038/srep21917.
Sperling R A et al., (2011) Toward defining the preclinical stages of Alzheimer's disease: Recommendations from the National Institute on Aging and the Alzheimer's Association workgroup. Alzheimers Dementia; 7(3):280-292.
Sturchler-Pierrat C et al., (1997) Two amyloid precursor protein transgenic mouse models with Alzheimer disease-like pathology. Proc. Natl. Acad. Sci. USA; 94(24):13287-13292.
Thorgrimsen L et al., (2003) Whose Quality of Life Is It Anyway? The Validity and Reliability of the Quality of Life-Alzheimer's Disease (QoI-AD) Scale. Alzheimer Dis. Assoc. Disord.; 17:201-208.
Vlassenko A G et al., (2012) PET amyloid-beta imaging in preclinical Alzheimer's disease Biochim. Biophys. Acta; 1822(3):370-379.
Weigand S D et al., (2011), Transforming cerebrospinal fluid Abeta42 measures into calculated Pittsburgh Compound B units of brain Abeta amyloid. Alzheimer's Dement: J. Alzheimer's Assoc., 7: 133-141.

All references, e.g., a scientific publication or patent application publication, cited herein are incorporated herein by reference in their entirety and for all purposes to the same extent as if each reference was specifically and individually indicated to be incorporated by reference in its entirety for all purposes. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method for the prevention of Alzheimer's disease in a patient at risk of developing clinical symptoms of Alzheimer's disease, said method comprising administering a prophylactically effective amount of the compound N-(6-((3R,6R)-5-amino-3,6-dimethyl-6-(trifluoromethyl)-3,6-dihydro-2H-1,4-oxazin-3-yl)-5-fluoropyrin-2-yl)-3-chloro-5-(trifluoromethyl)picolinamide, or a pharmaceutically acceptable salt thereof.

2. The method according to claim 1, wherein the patient at risk of developing clinical symptoms of Alzheimer's disease carries a genetic predisposition for the development of the clinical symptoms of Alzheimer's disease or has Down syndrome.

3. The method according to claim 2, wherein the patient carries a genetic predisposition for the development of the clinical symptoms of Alzheimer's disease and the genetic predisposition is:
(i) a mutation in the gene for amyloid precursor protein, presenilin-1 or presenilin-2; or
(ii) the presence of one or two copies of the ApoE4 allele.

4. The method according to claim 3, wherein the patient at risk of developing clinical symptoms of Alzheimer's disease carries one or two copies of the ApoE4 allele.

5. The method according to claim 1, wherein the patient carries one copy the ApoE4 allele.

6. The method according to claim 4, wherein the patient carries two copies of the ApoE4 allele.

7. The method according to claim 1, wherein the patient is amyloid-positive.

8. The method according to claim 7, wherein the amyloid-positivity is determined by PET or CSF measurement.

9. The method according to claim 3, wherein th patient is between 60 and 75 years of age.

10. The method according to claim 1, wherein the compound is used at a daily dose which results in at least a 70% lowering of Aβ 1-40 in CSF following two weeks of compound exposure.

11. The method according to claim 1, wherein the compound is used at a daily dose which results in at least a 50% lowering of Aβ 1-40 in CSF following two weeks of compound exposure.

12. The method according to claim 1, wherein the compound is used at a dose of 15 mg per day.

13. The compound, or a pharmaceutically acceptable salt thereof, for the use according to claim 1, wherein the compound is used a dose of 50 mg per day.

14. The method according to claim 1, wherein the compound is in free form.

15. (canceled)
